(19) United States
(12) Patent Application Publication
(10) Pub. No.: US 2004/0033932 A1
(43) Pub. Date: Feb. 19, 2004

(54) Myc targets

(76) Inventors: Rogier Versteeg, Amerongen (NL); Hubertus Nicolaas Caron, Heemskerk (NL)

Correspondence Address: TRASK BRITT, P.O. BOX 2550, SALT LAKE CITY, UT, 84110, US

(21) Appl. No.: 10/293,222

(22) Filed: Nov. 12, 2002

Related U.S. Application Data
(63) Continuation of application No. PCT/NL01/00361, filed on May 11, 2001.

(51) Int. Cl. A61K 31/00; C12Q 1/68

(52) U.S. Cl. 514/1; 435/6

(57) ABSTRACT

The invention discloses a catalog of targets of the myc oncogene family identified by Serial Analysis of Gene Expression. The invention also discloses a method for analyzing myc downstream targets in view of the full context of myc induced changes in gene expression. The invention further discloses a method for the treatment of cancer comprising modulating a myc-dependent downstream gene capable of supporting an essentially neoplastic characteristic of the cancer. The invention additionally discloses a method of screening to identify drugs that interfere with myc downstream effects.

### CROSS-REFERENCE TO RELATED APPLICATIONS

This application is a continuation of PCT/NL01/00361, filed May 11, 2001, designating the United States of America, corresponding to WO 01/85941 (published Nov. 15, 2001), the contents of which are incorporated herein in its entirety.

### TECHNICAL FIELD

The invention relates to the treatment and diagnosis of cancer and to the development of drugs for the treatment of cancer.

### BACKGROUND

The normal healthy organism maintains a carefully regulated balance that responds to specific needs of the body. In particular, the balance between the creation or multiplication of new cells and the death of superfluous cells is well maintained. However, the exquisite controls that regulate cell multiplication occasionally break down and a cell will grow and divide although the body has no further need for cells of its type. The cell essentially becomes neoplastic whereas there is no real need for the cell. When the descendants of such a cell inherit the propensity to multiply without regulation, the result is a clone of cells that are able to expand indefinitely. Ultimately, a mass called a tumor may be formed by the clones of unwanted cells and the affected individual may develop the beginning of cancer.

In older individuals, neoplasias or tumors arise with great frequency, but often pose little risk to the host in which the tumor is located because the tumor is localized and generally called benign. Tumors may become life-threatening if the tumor spreads through the body. Such tumors are often called malignant and are a further development of cancer.

The major characteristics that differentiate malignant tumors from benign tumors are the invasiveness and spread of the malignant tumor. Malignant tumors do not remain localized and encapsulated, but instead the malignant tumors may invade surrounding tissues, get into the body's circulatory system, and proliferate in areas away from the site of the malignant tumor's original appearance. The spread of malignant tumor cells and establishment of secondary areas of growth is called metastasis, wherein the multiplying and spreading malignant cells have acquired the ability to metastasize.

Since apparently benign tumors may progress to malignancy and early stages of malignant tumors are hard to identify, pathologists are rarely sure how malignancies begin. The cells of malignant tumors have a tendency to lose differentiated traits, to acquire altered chromosomal compositions, and to become essentially metastatic where the malignant cells become invasive and spread.

A wealth of knowledge has been developed about the genetic events that transform a normally regulated cell into a cell that grows without responding to controls. The genetic events are generally not inherited through the gametes, but rather are changes in the DNA of somatic cells. The principal type of change is the alteration of pre-existing genes into oncogenes, whose products cause the inappropriate cell growth. Thus, DNA alteration is at the heart of cancer induction and much focus has been given in scientific research to elucidating the causative genetic events. For example, the members of the myc oncogene family play an important role in cancer. In response to the frequency of genetic alterations of myc genes in human cancers (Dang and Lee, 1995), it is estimated that there are approximately 70,000 cancer deaths in the United States per year which are associated with changes in myc genes or in their expression. Three members of the myc-family, N-myc, c-myc and L-myc are rearranged, amplified, mutated and/or over-expressed in many cancers of the lung, breast colon, in various leukemias and brain tumors.

The c-myc gene is expressed in a wide variety of tissues and tumors, while N-myc expression is largely restricted to embryonic tissues, pre-B cells and neuroendocrine tumors. N-myc is amplified in human neuroblastoma, small cell lung carcinoma and strongly expressed in Wilms' tumors and retinoblastoma. Neuroblastoma is a childhood tumor with a highly variable prognosis. Approximately 20% of neuroblastomas have N-myc amplification and the tumors follow a very aggressive course (Schwab et al., 1983, Seeger et al., 1985). Over-expression of transfected N-myc genes in neuroblastoma cell lines strongly increases proliferation rates (Bernards et al., 1986; Lutz et al., 1996). Transgenic mice overexpressing N-myc in neural crest-derived tissues show a frequent development of neuroblastoma (Weis et al., 1997). Numerous comparable observations have implicated c-myc and L-myc in the pathogenesis of many other tumor types (Cole, 1986; Marcu et al., 1992; Henrikson and Luscher, 1996).

The myc-family members are transcription factors with a basic/helix-loop-helix/leucine zipper (bHLHzip) domain. Heterodimers of myc and MAX proteins bind to the E-box motif CACGTG and activate target gene transcription (Blackwood et al., 1992; Alex et al., 1992; Ma et al., 1993). The limited number of identified target genes has precluded the identification of myc downstream pathways. However, many experiments have suggested a role for myc genes in cell cycle control, metastasis, blocking of differentiation, apoptosis and proliferation rate (Henriksson and Luscher, 1996; Dang, 1999; Schmidt, 1999). Phenotypic analysis of mammalian cell lines and drosophila mutants with impaired myc function suggests a role for myc genes in cellular growth. Inactivation of both c-myc alleles in rat fibroblasts resulted in a 2- to 3-fold reduced growth rate (Mateyak et al., 1997). Impaired in vivo expression of drosophila d-myc retards cellular growth and results in adult flies half the normal size. A role for myc genes in growth regulation is further in line with the myc gene's affect on the cell cycle. Inactivation of c-myc in rat fibroblasts prolonged the G1 and G2 phases of the cell cycle, but not the S phase. High expression of c-myc or N-myc in human cells accelerated transition through the G1-phase (Steiner et al, 1995; Lutz et al., 1996). The same effect was found in drosophila cells, where reduced d-myc activity increased the length of the G1 phase, while increased d-myc expression enhanced transition through G1 (Johnston et al., 1999).

Many studies have implicated the expression of myc-family oncogenes in metastasis. In several tumor series, a correlation between the expression of myc genes and the occurrence of metastases exists. This was observed for c-myc in breast cancer, bone tumor and colon cancer (Sierra et al., 1999, Gamberi et al., 1998, Kakisako et al., 1998). Experimental systems confirm a direct relationship between expression of myc genes and metastatic capacity. For instance, human-melanoma cells overexpressing c-myc were more metastatic than control melanoma cells (Schlagbauer-Wadl et al., 1999). However, the mechanism of how expression of myc genes increases the metastatic potential of tumor cells is unknown.

Several direct targets of c-myc, as well as a series of indirectly induced genes, have been identified, but no links between the genes have been found. The incidental and isolated characteristics of the observations preclude the identification of a comprehensive and integrated view of the cellular effects of myc genes in cancer. Examples are prothymosin a (Eilers et al., 1991), ornithine and decarboxylase (Bello-Femandez et al., 1993), the embryonically expressed ECA39 gene (Benvenisty et al., 1992), translation initiation factors elF-4E and eIF-2-alpha (Jones et al., 1996; Rosenwald et al, 1993), the CAD gene (Boyd et al., 1997), the DEAD-box gene MrDb (Grandori et al., 1996) and nucleolin (Greasley et al., 2000). Effects on cyclins and other cell cycle regulators depend on cell types and conditions. Induction of cyclin D1 or tyrosine protein phosphatase cdc25A were found in some model systems (Galaktionov et al.; 1996, Amati et al.; 1998; Philipp et al.; 1994; Daksis et al.; 1994; Solomon et al., 1995). Also, induction of cyclin E and A expression has been reported (Jansen-Durr et al.; 1993; Hanson et al., 1994). The c-myc targets prothymosin a and ornithine decarboxylase are also induced by N-myc, but it is unknown whether c-myc and N-myc share all their targets (Lutz et al., 1996). Thus, it is clear that myc genetic alterations are central and many alterations have been identified and mapped.

However, much less effort has been spent on elucidating the supporting physiological events that take place in the ever multiplying and spreading cancer cell. The fact that cells undergo genetic changes in their development into cancer cells is well understood. However, how these genetically changed cells recruit and adapt normal physiological mechanisms and events to support the essentially neoplastic character resulting in growth, invasion and spread is much less well understood. For example, the myc proteins are transcription factors which form dimers with the MAX protein and recognize the DNA sequence CACGTG. Further, very few target genes of the myc transcription factors have been identified. The identified myc targets do not permit a clear understanding of the pathway activated by myc proteins and, therefore, the biochemical role of these proteins in pathogenesis is a matter of speculation. Phenotypic observations of mammalian cell lines, transgenic mice and mutant drosophilas with aberrant expression of myc genes have suggested a role for myc genes in cell cycle control, metastasis, apoptosis, proliferation rate and cellular growth.

### SUMMARY OF THE INVENTION

The invention discloses that the myc oncogene family provides for the recruitment and adaptation of the normal physiological mechanisms and events to support the essentially neoplastic character of a cancerous cell resulting in growth, invasion and spread. The invention discloses a nucleic acid library comprising myc-dependent downstream genes or functional fragments thereof, wherein the myc-dependent downstream genes are essentially capable of supporting a neoplastic character of cancer such as growth, invasion or spread.

The phrase a “nucleic acid library” is defined herein as a collection of nucleic acid sequences comprising genes or functional fragments thereof which are downstream myc-dependent genes or functional fragments thereof. Up- or down-regulation of these genes is dependent on the presence of transcription factors encoded by the myc-family members. The nucleic acid sequences may be available in a vector which provides easy handling of the collection of nucleic acid sequences. The downstream myc-dependent genes or functional fragments may be identified by applying the SAGE (Serial Analysis of Gene Expression) technique. Initially, about 66,233 tags were identified, wherein each tag represents an mRNA transcript in a pair of N-myc-transfected and control-transfected neuroblastoma cell lines. Thus, 197 tags have been identified wherein each tag represents a transcript that is specifically induced and 85 tags that are suppressed by N-myc (Table 1). In an extension of these analyses to 79,100 transcripts, an additional series of transcripts were identified that are up- or down-regulated by N-myc (Table 2).

As used herein, the phrase “functional fragment” will be used to refer to a part of a myc-dependent downstream gene which contains the appropriate Tag-sequence to provide identification by the SAGE technique.

The invention discloses a nucleic acid library wherein the myc-dependent downstream genes comprise a nucleic acid essentially equivalent to a Tag sequence as shown in Table 1 or Table 2. The phrase “essentially equivalent” as used herein relates to Tag sequences that identify similar or related genes or fragments thereof. In one embodiment, a nucleic acid library is disclosed wherein the myc-dependent downstream genes encode a ribosomal protein, a protein related to protein synthesis, a protein related to metastasis, a glycolysis enzyme or a mitochondrial functional protein.

The invention also discloses a method for the treatment of cancer comprising modulating a myc-dependent downstream gene capable of supporting an essentially neoplastic character of the cancer, such as growth, invasion or spread. In one embodiment, the invention discloses a method wherein the myc-dependent downstream gene comprises a nucleic acid essentially equivalent to one of the Tag sequences shown in Table 1 or Table 2.

In one embodiment, the invention discloses downstream genes that are activated or repressed by N-myc in human neuroblastoma. The analysis of the expression level of more than 66,233 transcripts identified 199 up-regulated and 85 down-regulated transcripts in N-myc-expressing cells (Table 1) (Boon et al. 2001). An extension of these analyses to 79,100 transcripts identified an additional series of transcript tags that are up- or down-regulated by N-myc (Table 2). The results show that N-myc functions as a regulator of cell growth, facilitating neoplasia, by stimulating genes functioning in ribosome biogenesis and protein synthesis, as well as in mitochondrial electron transfer and ATP synthesis. Furthermore, many genes involved in cell architecture and cell-matrix interactions are down-regulated, facilitating invasion or spread of the neoplastic cell. A series of the N-myc-regulated genes are targets of c-myc regulation as well.

The invention herewith discloses a list of MYC-family target genes with a large number of identified targets which permits the identification of pathways induced or inhibited by myc-family genes. However, with the identification of the individual genes, several other practical applications are disclosed. The findings may be used for the development of new drugs, refined use of known drugs and recombinant technology.

In one embodiment, the invention discloses assays for high-throughput screening of drugs specifically inhibiting myc-proteins or myc-downstream pathway proteins. It is known that an individual cancer is caused by mutations in several oncogenes and/or tumor suppressor genes. Tumors of one and the same tissue can arise from different combinations of mutated oncogenes and tumor suppressor genes. The type and combination of gene defects determine the biology of the tumor cells, and thus the clinical behavior of the tumor. Future tumor therapies may be tailor made for the tumor of individual patients. Upon diagnosis, the type of oncogene activations in a tumor may be established and may guide the choice of therapy.

There are about 70,000 cancer deaths per year in the U.S. that are associated with defects in myc genes or over-expression of myc genes,(Dang and Lee 1995; Dang, 1999). There are currently no drugs specifically blocking the action of myc proteins. Such drugs may now be identified in high-throughput systems using target genes of the invention, where thousands of compounds may be tested for a specific inhibitory affect on myc proteins. These test-systems may require a very strict read-out system. An example of such a system has been used to identify inhibitors of the TP53 tumor suppressor protein and has been published (Komarov et al., 1999). A lacZ reporter construct was brought under the control of a promoter of a gene known to be induced by TP53. A mouse cell line harboring this construct was used to test 10,000 synthetic chemical compounds. The chemical compounds were added to tissue culture wells with the cells and the compounds that inactivated TP53 were identified by a reduced expression of the LacZ reporter gene.

The identification of the series of myc-target genes disclosed herein enables a sophisticated approach to identify myc-inhibitory drugs. Currently, there is no sensitive read-out system to identify these drugs. Some target genes of c-myc and/or N-myc have been identified, but their expression levels are only a few times modulated, making them essentially useless as a read-out system (Cole et al., 1999). As disclosed herein, the identification of a series of genes that are strongly induced or suppressed by N-myc and/or c-myc is described.

In one embodiment, expression of the osteonectin, or SPARC gene, is down-modulated by N-myc from 280 transcripts per 10,000 transcripts in SHEP-2 to 14 transcripts in SHEP-21N is disclosed. This 20-fold reduction is confirmed by Northern blot hybridization of mRNA from SHEP-2 and SHEP-21N (data not shown). Moreover, it is disclosed herein that at the protein level, SHEP-2 cells have more Osteonectin protein than SHEP-2 (data not shown). The Osteonectin protein is known as a secreted protein (Lane and Sage, 1994) which makes testing of N-myc inhibitory drugs using the Osteonectin protein feasible. Test-compounds can be added to SHEP cells wherein N-myc expression can be induced, e.g., by a tetracycline-inducible system. When N-myc expression is induced, Osteonectin protein levels will drop in these cells. If a compound is inhibitory for N-myc, the down-modulation of Osteonectin mRNA expression will be blocked and the cells will continue to secrete Osteonectin protein in the tissue culture medium. Thus, in a multi-well cell culture system, the wells with inactive compounds will have low Osteonectin production. Further, the wells with a high Osteonectin concentration will identify the compounds that inhibit N-myc function.

Elisa or other protein-based detection systems may be used to automatically detect the wells with high Osteonectin concentrations. Since other down-regulated genes like IGFBP7, collagen type 4a1 and syndecan 2 are identified to be down-regulated by myc, in addition to Osteonectin, these other down-regulated genes may also be used in assays. Similar assays can be designed the other way around using genes induced by N-myc or c-myc as a read-out. Variants of drug-testing systems may use the promoter elements of strongly N-myc-regulated genes and control expression of detectable proteins such as Green Fluorescent Protein. This further simplifies detection of myc-inhibitory drugs in high throughput systems.

Since it has been shown that many of the N-myc targets are also targets of c-myc, the findings may be used in assays to identify drugs against other members of the myc oncogene family. Inducible c-myc constructs will target the same genes as N-myc in many cell systems. Therefore, the list of target genes disclosed herein for N-myc and c-myc enables the semi-automatic testing of hundreds of thousands candidate drugs and the selection of compounds that are active against myc proteins and/or their downstream pathways.

The invention also discloses the application and further development of existing drugs for the specific treatment of patients with N-myc or c-myc-activated tumors, such as a method of treatment for myc-related cancer. Future cancer therapies may be designed to specifically inhibit oncogenes that are actually activated in the tumor of a specific patient. As disclosed herein, several known cytostatic or cytotoxic drugs may be used in patients with a tumor caused by activation of a myc gene.

The invention further discloses a method of treatment comprising using drugs that interfere with nucleophosmin. Nucleophosmin (B23) was identified as a major target of N-myc and c-myc induction. Analysis of a large series of human tumors and cell lines reveals that nucleophosmin mRNA levels also correlate in vivo with N-myc or c-myc expression. Nucleophosmin functions in ribosome biogenesis and nucleolar cytoplasmic transport of pre-ribosomal particles and the protein is known to be translocated from the nucleolus to the nucleoplasm by several cytotoxic drugs, such as actinomycin D, doxorubicin, mitomycin, toyocamycin, tubercidin, sangivamycin and mycophenolic acid (Yung et al., 1995, 1990; Chan et al., 1987; Chan 1988, 1992; Cohen and Glazer, 1985; Perlaky et al., 1997). Thus, treatment of cells with these drugs inhibits processing of ribosomal RNA, protein synthesis and leads to cell death. Several of these drugs are clinically tested and/or applied for treatment of cancer patients.

Since members of the myc oncogene family induce nucleophosmin mRNA and protein expression, nucleophosmin is an attractive target in the treatment of tumors with overexpression of a myc family member. Thus, fundamental new opportunities for the use of the above mentioned drugs and their analogs exist. The drugs were clinically tested or used on unselected patient series without knowledge of the drug's possible effect on myc-activated tumors. Presumably, the patient series included patients with and without the involvement of myc genes in the tumors. Actinomycin D was used in Wilms' tumor and rhabdomyosarcoma treatment. It was found that some patients reacted better to therapy than others. A high number of Wilms' tumor and rhabdomyosarcoma patients have high N-myc or c-myc expression (Nisen et al., 1986). Thus, it may be tested whether actinomycin D is specifically effective against N-myc or c-myc-expressing tumors, wherein actinomycin D is ineffective against tumors that have no myc activations.

Based on these findings, a more specific use of actinomycin D is disclosed. Several other drugs are also effective against nucleophosmin (as described herein). Improvement of these drugs by the development of less toxic analogs with more specific anti-nucleophosmin effects is a strategy disclosed herein to design specific anti-myc drugs. For instance, since toyocamycin is highly toxic (Wilson, 1968), analogs thereof can be tested and further developed for specific use in patients with myc-activated tumors.

The invention also discloses inhibitors of extracellular and transmembrane proteins. The list of genes induced by N-myc and/or c-myc includes many genes coding for secreted or cell surface proteins. These proteins are excellent targets for drugs because they are readily accessible and may offer targets to specifically inhibit growth or metastasis of tumor cells with an activation of myc-family members. Examples of potential targets are basigin and Plasminogen Activator Inhibitor type 1 (PAI) (Table 1, No. 112). Basigin (also referred to as EMMPRIN, extracellular matrix metalloproteinase inducer) (Table 1, No. 97) is a member of the immunoglobulin family that is present on the surface of tumor cells and stimulates nearby fibroblasts to synthesize matrix metalloproteinases (Guo et al., 1998). Since metalloproteinases are known to promote the degradation of matrix and promote metastasis, drugs that inhibit basigin would be able to prevent metastasis of tumors with high expression of myc-family genes. Drugs inhibiting metalloproteinase are known and may be applied to specifically treat tumors by activating myc-family members.

Plasminogen activator inhibitor type-i (PAI-1) is a major physiological inhibitor of fibrinolysis and matrix turnover. The down-modulation of PAI-1 by N-myc described herein may increase matrix turnover and promote cell motility and metastasis. Many compounds have been clinically and experimentally tested for regulation of PAI, such as 15-deoxy-Deltal 2, 14-prostaglandin J2 (15d-PGJ2) (an activating ligand for the transcription factor PPARg) has been shown to augment PAI-1 mRNA and protein expression (Marx et al., 1999). As disclosed herein, these drugs may be specifically used to prevent metastasis of N-myc or c-myc-expressing tumors. Furthermore, transmembrane proteins induced by N-myc and/or c-myc proteins may be used as a target of therapeutic drugs, such as antibodies conjugated with cytotoxic drugs.

The invention further discloses molecular diagnosis of tumors. A first integral description of target genes of myc-family oncogenes is disclosed wherein several functional categories are disclosed. However, in the analysis of fresh tumors for N-myc activation, it was observed that not all these genes or categories of genes are up-regulated in all tumors. This finding suggests that additional defects or factors may affect the range of genes that are induced or repressed by myc oncogenes in individual tumors which is likely to be of importance for the biology of the tumor.

The detailed analysis of up-regulated/suppressed genes in tumors for activation of a myc-family member is clinically relevant. A tumor with up-regulation of genes involved in protein synthesis may differ from a tumor with up-regulation of genes involved in oxidative phosphorylation. This is important for selecting the appropriate treatment modality for a tumor. The full list of N-myc and/or c-myc downstream targets thus discloses an important means to classify tumors for optimal therapeutic regimens. This finding may be applied in the development of diagnostic kits that measure the activation or inactivation of key-downstream targets of myc-family oncogenes. Such diagnostic tools are may be provided to guide the optimal therapy.

Further, the invention discloses non-invasive diagnosis of tumors. Activation of specific oncogenes is currently established by the analysis of a surgically removed tumor specimen. As surgery is a burden on the patient, expensive and not without risk, non-invasive methods to monitor the oncogene status of tumors are desirable. The inventory of genes for secreted proteins that are induced or suppressed by N-myc may be used to determine the status of myc expression by the analysis of serum markers. Further, serum levels of these genes may be used to monitor tumor growth, reaction to therapy and occurrence of relapses. The results described herein show that many candidate proteins, e.g., osteonectin (reduced from 280 to 14 tags/i 0,000 tags), macrophage migration inhibitory factor (Table 1, No. 92) (induced from 1.1 to 14.4 tags/10,000 tags) and Plasminogen activator inhibitor type 1 (PAI-1), may be used as serum markers to aid in the biological classification of tumors. Recently, serum levels of PAI-1 were analyzed in a series of head and neck tumors and found to correlate with tumor stage (Strojan et al., 1998). Therefore, PAI-1 and other secreted proteins were identified to be good candidates to monitor the status of myc genes in a tumor and to follow the growth and response of therapy on myc-induced tumors.

The invention further discloses enhancement of cellular protein synthesis machinery for production purposes. Eukaryotic cells may be used to produce recombinant proteins, such as drugs. The discovery that N-myc and c-myc induce essential components of the protein synthesis machinery may be applied to boost production of recombinant proteins in cell systems. The invention discloses the use of cells with a high expression of endogenous or transfected myc genes to optimize the yields of recombinant proteins, such as antibodies, hormones or other proteins with therapeutic or commercial value.

The invention further discloses a method to identify a substance capable of interfering with n-myc or n-myc-induced modulation of transcripts and/or proteins, comprising providing a cell with n-myc activity or a nucleic acid encoding n-myc activity and determining the modulation of the transcripts and/or proteins in the presence of the substances. As disclosed herein, the modulation of transcripts and/or proteins by n-myc can be either up- or down-regulated.

### BRIEF DESCRIPTION OF THE DRAWINGS

FIG. 1. Northern blot analysis of N-myc downstream target genes. Equal amounts of total RNA from exponentially growing SHEP-2 and SHEP-21N cells were loaded. Northern blots were hybridized with probes for the 23 indicated N-myc targets.

FIG. 2. Level of induction of the 56 ribosomal protein genes identified as N-myc targets (p<0.01) in SHEP-21N cells. FIG. 2A: Fold induction by N-myc in SHEP-21N as a function of the basic expression levels in SHEP-2. X-coordinate: basic expression level in SHEP-2 normalized per 10,000 tags. Y-coordinate: fold induction in SHEP-21N. FIG. 2B: Increase of the same 56 ribosomal protein genes in N-myc-amplified neuroblastoma N159 as a function of the basic expression level in N-myc single copy neuroblastoma N52. X-coordinate: expression level in N52 normalized per 10,000 tags. Y-coordinate: Fold increase in N159 relative to N52.

FIG. 3. Time-course analysis of N-myc and downstream target gene induction in SHEP-21N cells. SHEP-21N cells were treated for 24 hours with 10 ng/ml tetracycline, washed and grown for an additional 36 hours without tetracycline. Cells were harvested at 0 hour, 8 hours and 24 hours of tetracycline treatment. Subsequent samples were taken at 1 hour, 2 hours, 4 hours, 8 hours, 10 hours, 12 hours, 24 hours and 36 hours after removal of the antibiotic. FIG. 3A: Northern blot analysis of total RNA at indicated time points. FIG. 3B: Western blot analysis of N-myc protein at indicated time points. 10 mg of total protein samples of the time-course experiment were fractionated through a 10% SDS-PAGE gel, blotted on Immobilon membrane and probed with a monoclonal anti-N-myc antibody.

FIG. 4. Nucleolin and Nucleophosmin protein expression and total RNA content of SHEP-2 and SHEP-21N. FIG. 4A: Western blot analysis of nucleolin, N-myc and nucleophosmin protein expression. Total cell extracts (10 μg) were fractionated through an acrylamide gel, blotted and probed with polyclonal antibodies against nucleolin (upper panel), monoclonal antibodies against N-myc (middle panel) and nucleophosmin (lower panel). Control cell lines IMR32 and SK-N-FI have high and low expression, respectively, of N-myc, nucleolin and nucleophosmin. FIG. 4B: Total RNA content of SHEP-2 and SHEP-21N. RNA was isolated from ten samples of 10⁶ cells of each cell line and photospectromerically analyzed. Error bars give the S.D.

FIG. 5. Northern blot analysis of total RNA from neuroblastoma cell lines and tumors. Filters were hybridized with indicated probes. RNA quantification was checked by ethidium bromide staining and the 28S band is shown. FIG. 5A: panel of 21 neuroblastoma cell lines. FIG. 5B: Panel of 16 fresh tumors. Tumors in lanes 1-9 are N-myc-amplified.

FIG. 6. Northern blot analysis of induction of N-myc target genes in a c-myc-transfected melanoma cell line. Clone 3 is a c-myc-transfected clone of the IGR39D melanoma cell line. Equal amounts of total RNA of IGR39D and clone 3 were loaded. Filters were hybridized with the indicated probes.

### DETAILED DESCRIPTION

The members of the myc oncogene family play an important role in cancer. The frequency of genetic alterations of myc genes in human cancers (Dang and Lee, 1995) has allowed an estimation that approximately 70,000 cancer deaths occur per year in the United States and are associated with changes in myc genes or in myc gene expression. Three members, N-myc, c-myc and L-myc are rearranged, amplified, mutated and/or over-expressed in many cancers of the lung, breast, colon, in various leukemias and brain tumors. The myc proteins are transcription factors that form dimers with the MAX protein and recognize the DNA sequence CACGTG. Very few target genes of the myc transcription factors have been identified thus far, and the identified targets do not permit a clear understanding of the pathway activated by myc proteins. Therefore, the biochemical role of these proteins in pathogenesis is a matter of much speculation. Phenotypic observations of mammalian cell lines, transgenic mice and mutant drosophilas with aberrant expression of myc genes have suggested a role for myc genes in cell cycle control, metastasis, apoptosis, proliferation rate and cellular growth.

To identify the downstream pathways of the myc genes, the SAGE (Serial Analysis of Gene Expression) technique was utilized. Initially, about 66,233 tags were identified, each representing an mRNA transcript in a pair of N-myc-transfected and control-transfected neuroblastoma cell lines. Thus, 197 tags have been identified, wherein each tag represents a transcript that is specifically induced and 85 tags have been identified that are suppressed by N-myc (Table 1). By extending these analyses to 79,100 transcripts, an additional series of transcripts were identified that are up- or down-regulated by N-myc (Table 2). N-myc appears to induce the expression of many ribosomal protein genes that are involved in ribosomal RNA synthesis, ribosome biogenesis and genes involved in translation and protein maturation. This indicates that a major function of N-myc is the enhancement of the protein synthesis machinery of the cell. Furthermore, a striking induction of genes involved in glycolysis, electron transport and ATP synthesis was observed in the mitochondria. This suggests an increased capacity of the cellular energy production mechanism.

Another set of N-myc targets is involved in cellular adhesion, matrix formation, invasive capacity and cytoskeletal architecture. This data explains the increased metastatic potential associated with myc-expressing tumor cells. Furthermore, a set of genes is induced or suppressed with a role in transcription, chromosome condensation and signal transduction. Finally, a series of genes are identified for which only a short cDNA sequence is known (Ests) and some SAGE transcript tags were identified without a gene assignment. These unidentified genes may be important components of the N-myc downstream pathway. Many of the downstream targets of N-myc appear to be targets of the c-myc oncogene as well. Therefore, the data presented herein represents an inventory of target genes of the myc oncogene family. As these genes mediate the tumorigenic effects of myc-family oncogenes, the genes offer the opportunity to identify new drugs that inhibit myc proteins or myc downstream pathways. Further, the genes represent a range of potential target genes to inhibit or kill tumor cells that express members of the myc-family of oncogenes.

Results.

SAGE libraries of N-myc-transfected neuroblastoma cell lines.

To identify the downstream target genes of N-myc, the SAGE technique was used on an N-myc-transfected neuroblastoma cell line. The SHEP cell line has no N-myc amplification and expression, and no c-myc expression. A tetracycline-dependent N-myc expression vector was introduced in these cells, resulting in the SHEP-21N clone (Lutz et al., 1996). The SHEP-21N cells have constitutive exogenous N-myc expression that can be switched off by tetracycline. N-myc expression in the SHEP-21N cells was shown to increase the rate of cell division, shorten the GI phase of the cell cycle and to render the cells more susceptible to apoptotic triggers (Lutz et al., 1996; Fulda et al., 1999).

Two SAGE libraries were constructed; one from SHEP-21N cells expressing N-myc and one from the SHEP-2 control cells. The SHEP-2 clone was transfected with the empty expression vector. About 44,674 transcript tags were sequenced from SHEP-2 and 21,559 transcript tags were sequenced from the SHEP-21N library. Comparison of the two SAGE libraries yielded 199 significantly (p<0.01) up-regulated tags in N-myc-expressing cells, with induction levels of up to 47-fold (Table 1, section 1). Another 85 tags were significantly down regulated. Further sequencing of the SHEP-21N library from 21,559 tags to 34,426 tags yielded another series of transcript tags that were either up- or down-regulated by N-myc (p<0.01) (Table 2). Table 2 describes these tags and the most likely gene assignment that corresponds to the tags. The transcripts corresponding to the tags were identified using a computer program (Caron et al., 2001) and using the SAGEmap database from CGAP/NCBI (Lal et al., 1999). Seven groups of N-myc-regulated genes are disclosed.

N-myc targets 1: ribosomal protein genes.

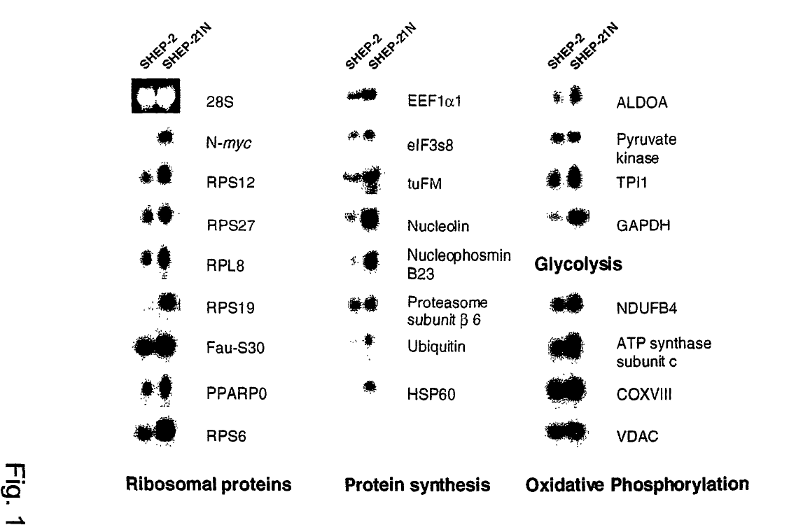

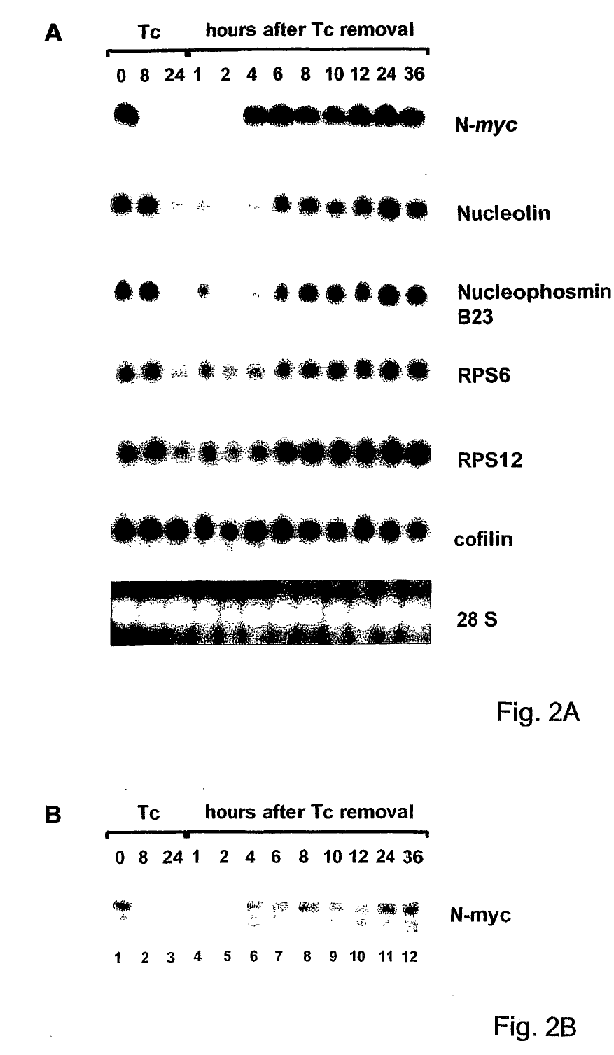

The first functional group includes 61 ribosomal protein genes that were induced up to 47-fold (p<0.01, Table 1, section 1). The 61 proteins represent about 75% of the human ribosomal proteins (Wool et al., 1996). Seven of the induced genes were selected for further analysis. Northern blots with equal amounts of total RNA from SHEP-2 and SHEP-21N cells were hybridized with probes for the ribosomal proteins S 12, S27, Fau-S30, L8, S6, S 19 and the ribosomal phosphoprotein P0 (PPARP0) (FIG. 1). The seven genes were induced by N-myc. The total amount of tags found for ribosomal protein mRNAs comprises about 4% of all tags in SHEP-2. This fraction increased to 10% in SHEP-21N. The level of induction of individual ribosomal protein genes is a function of their basal expression levels in SHEP-2. Highly expressed genes are less induced than genes with a low basic expression in SHEP-2 (FIG. 2A). The results indicate that N-myc induces, directly or indirectly, the mRNA expression level of the majority of ribosomal proteins.

2N-myc targets 2: genes functioning in ribosome biosynthesis and protein synthesis.

A second functional group of 26 tags corresponds to genes with a distinct role in protein synthesis and turnover, notably ribosome biogenesis, mRNA translation, protein maturation and degradation.

The induction of nucleophosmin (B23) (Table 1, nos. 67 and 83) was observed. Northern blot analysis confirmed the induction (FIG. 1) to a level stronger than suggested by the tag frequencies. Nucleophosmin is a highly abundant nucleolar protein that processes ribosomal RNA by cleavage of the 5′ end of the 5.8S pre-rRNA (Savkur et al., 1998). Nucleophosmin also functions in assembly and nuclear-cytoplasmic shuttling of pre-ribosomal particles (Borer et al., 1989; Olson et al., 1991; Szebeni et al, 1999). Nucleophosmin is the target of recurrent chromosomal translocations in lymphomas and leukemia (Morris et al., 1994; Redner et al., 1996; Pandolfi, 1996). The SAGE libraries were also analyzed for other genes implicated in the process of nucleophosmin's role in ribosome biogenesis. Nucleolin, which also has two tags attributed to alternative transcripts (Table 1, nos. 87 and 88), is induced from 2.5 to 5.6 tags per 10,000 in total (p=0.044). This induction was confirmed by Northern blot analysis (FIG. 1). Nucleolin is also a highly abundant nucleolar protein that binds to nucleophosmin (Tujeta and Tujeta, 1998; Ginisty et al., 1999). Nucleolin is probably a rate-limiting enzyme for the first step in the processing of the pre-ribosomal RNA to mature 18S rRNA (Gistiny et al, 1998). Nucleolin is furthermore involved in the assembly of pre-ribosomal particles and their nucleo-cytoplasmic transport that interacts with 18 ribosomal proteins (Bouvet et al., 1998), sixteen of which are induced by N-myc. The induction of nucleolin and nucleophosinin by N-myc suggests that in addition to ribosomal proteins, ribosomal RNA and ribosome biosynthesis are also targets of N-myc stimulation.

Tags corresponding to three translation initiation factors and five translation elongation factors were also induced. The initiation factors are eukaryotic translation initiation factor 3 subunit 8 (eIF3s8) (Table 1, No. 81) and subunit 3 (Table 1, No. 78), and eukaryotic translation initiation factor 4B (Table 1, No. 72). Elongation Factor 1 (EEF1), responsible for delivery of aminoacyl-tRNA to the ribosome, is a heterotrimer including the subunits alpha/beta/gamma or alpha/delta/gamma. The tags for the subunits alpha, delta and gamma are induced 9- to 11.4-fold in SHEP-21N (Table 1, Nos. 69, 66 and 70). Elongation Factor 2, which promotes the translocation of the nascent polypeptide chain from the A- to the P-site of the ribosome, is also induced (Table 1, No. 79). The mitochondrial elongation factor Tu (tuFM), which delivers aminoacyl-tRNA to the mitochondrial ribosomes, is 12.4 times up-regulated (Table 1, No. 64). Northern blot analysis of SHEP-21N and SHEP-2 confirmed the induction of eIF3s8, EEF1a1 and tuFM (FIG. 1). This data further supports a role for N-myc as a regulator of protein synthesis.

Protein synthesis also includes the steps of maturation and routing. The nascent polypeptide-associated complex (NAC) alpha mRNA was induced in N-myc-expressing cells (Table 1, No. 77). NAC protects nascent polypeptide chains of cytosolic proteins from inappropriate translocation to the endoplasmatic reticulum (Wiedmain et al., 1994). Induction of the chaperones HSP60 and HSP90 further suggested an increased cellular capacity for protein folding and maturation (Table 1, Nos. 65, 68, 80 and 82). HSP60 is implicated in mitochondrial protein import and macromolecular assembly. HSP90 is involved in the folding of a signaling molecule including steroid-hormone receptors, kinases and the refolding of misfolded proteins. Northern blot analysis confirmed the induction of HSP60 (FIG. 1). The cellular capacity for protein degradation was possibly induced which was suggested by the increased tag frequencies for three ubiquitin pathway proteins (Table 1, Nos. 62, 73 and 76) and five proteasome subunits (Table 1, Nos. 63, 71, 74, 75 and 84). Northern blot analysis confirmed the higher expression level of proteasome subunit b type 6 in SHEP-21N cells (FIG. 1).

N-myc targets 3: glycolysis genes.

A third group of N-myc-induced genes encoded key-enzymes in the glycolytic pathway (Table 1, section 4). Tags for aldolase A fructose-biphosphate (ALDOA), triosephosphate isomerase 1 (TPI1), glyceraldehyde-3-phosphate dehydrogenase (GAPDH) and pyruvate kinase are increased (Table 1, Nos. 133, 135 and 132). Other induced mRNAs encode for the metabolic enzymes 3-phosphoglycerate dehydrogenase involved in the synthesis of serine and sorbitol dehydrogenase that oxidizes sorbitol to fructose. Aldehyde dehydrogenase 1 functions in ethanol metabolism. Northern blot analysis confirmed the mRNA induction of ALDOA, pyruvate kinase, TPI1 and GAPDH (FIG. 1). These data implicate glycolysis proteins as a target of N-myc stimulation.

N-myc targets 4: Mitochondrial electron carriers and ATP synthethase.

SHEP-21N shows induction of a series of tags corresponding to genes with a role in oxidative phosphorylation in the mitochondria (Table 1, section 5). Seventeen tags are significantly induced. Five of the induced genes are mitochondrially-encoded. (See, Welle et al., 1999.)

The oxidation of NADH and FADH2 by electron transfer to O₂ is performed by three protein complexes of the respiratory chain, NADH-dehydrogenase, ubiquinol-cytochrome c reductase and cytochrome c oxidase. These large complexes establish a proton gradient across the mitochondrial inner membrane and drive the synthesis of ATP by the F-type ATP synthase complex. N-myc induces a series of subunits of all four enzyme complexes.

Four NADH dehydrogenase subunits, subcomplex 4 (Table 1, No. 136; NDUFB4), subcomplex 7 (Table 1, No. 138) and the mitochondrially encoded subunits 4/41 and 3 (Table 1, Nos. 150 and 151) are induced. The induction of NDUFB4 was confirmed by Northern blot analysis (FIG. 1).

One subunit of the ubiquinol-cytochrome c reductase complex was induced (Table 1, No. 136). Furthermore, subunits II, III and VIII of cytochrome c oxidase were induced in N-myc-expressing cells. Induction of the subunit VIII (COXVIII, Table 1, No. 149) was confirmed by Northern blot analysis.

N-myc induces the transcripts of subunits 6/8 of the F0 segment and of two isoforms of subunit 9 (or c) of the F0 segment of the stalk of the F-type ATP synthase (Table 1, Nos. 137,148, and 152). ATPase subunits 6 and 8 are encoded on an overlapping mitochondrial transcript.

Several other proteins with a role in mitochondrial function are also up-regulated (Table 1, section 5). The voltage-dependent anion channel (VDAC, Table 1, No. 143) was induced 5-fold, which was confirmed by Northern blot analysis (FIG. 1). VDAC forms a mitochondrial outer membrane channel that allows diffusion of small hydrophylic molecules and plays a major role in apoptosis as it can transfer cytochrome c to the cytoplasm, resulting in caspase 9 activation.

Glutathione peroxidase 4 and glutathione S-transferase p are also strongly induced (Table 1, Nos. 139 and 144). Glutathione readily accepts electrons and may serve as a scavenger for hydrogen peroxide and organic peroxides, the inevitable artifacts produced by the electron transport chain of the mitochondria. The reaction is catalyzed by glutathione peroxidase.

N-myc targets 5: genes with a role in cell motility and metastasis.

A large group of tags that are either induced or suppressed by N-myc belong to genes with a role in cell motility and cell-matrix interactions (Table 1, section 3). These genes encoded cytoskeletal proteins, cell surface proteins, adhesion molecules and extracellularly secreted proteins with a role in cellular matrix architecture and turnover. Ten tags for genes in this category were significantly induced. Another 29 tags in this category are significantly down-modulated. Examples of down-regulated genes include Collagen types Ia1 (Table 1, Nos. 100, 108 and 109), type IVa1 (Table 1, No. 115) and type XVIIIa1 (Table 1, No. 116), fibrillin (Table 1, No. 121), syndecan 2 (Table 1, No. 126), fibronectin (Table 1, No. 122) and Osteonectin (SPARC) (Table 1, Nos. 118, 123 and 125). Osteonectin is down-modulated from 208 to 14 tags per 10,000 tags. Down-modulation of Osteonectin, syndecan 2, collagen IVa1 and Plasminogen activator inhibitor type 1 (Table 1, No. 112) were confirmed by Northern blot analysis (data not shown). The down-modulation of these genes suggests that N-myc can reduce the adherence of cells to the cellular matrix and, therefore, induce the motility of the cells which is in line with an enhanced metastatic potential of myc-expressing tumor cells.

N-myc targets 6: other genes.

Another group of genes affected by N-myc is formed by signal transduction proteins, transcription factors, chromatin factors, cyclins and other regulatory proteins. This group includes 66 significantly induced transcripts (Table 1, section 6). Examples are NM23A, NM23B, HMG I-Y and zinc finger protein 6 (Table 1, Nos. 171,214,162 and 218). Induction of HMG I-Y, NM 23A and NM23B was confirmed by Northern blot analysis (data not shown). Another group of genes of regulatory proteins or enzymes were down-modulated by N-myc (Table 1, section 6). Examples are Insulin-like growth factor binding protein 7 (IGFBP7) (Table 1, No. 52) and zinc-finger protein 216 (Table 1, No. 248). Northern blot analysis confirmed down-regulation of IGFBP7 in SHEP-2 cells as compared to SHEP-21N cells (data not shown).

N-myc targets 7: Anonymous genes (Ests).

A series of anonymous genes for which only a partial cDNA sequence is known (expressed sequence tags or Ests) are induced or down-modulated by N-myc (Table 1, section 7). The function of these genes is unknown, but the finding that the genes are targets of myc regulation mark them as potentially important genes with a role in cancer.

Tags of unidentified targets of N-myc.

For several tags that were differentially represented in the SHEP-2 and SHEP-21N libraries, the corresponding genes have not yet been identified (Table 1, section 8). These tags belong to genes that are induced or suppressed by N-myc.

N-myc activates downstream targets within 4 hours.

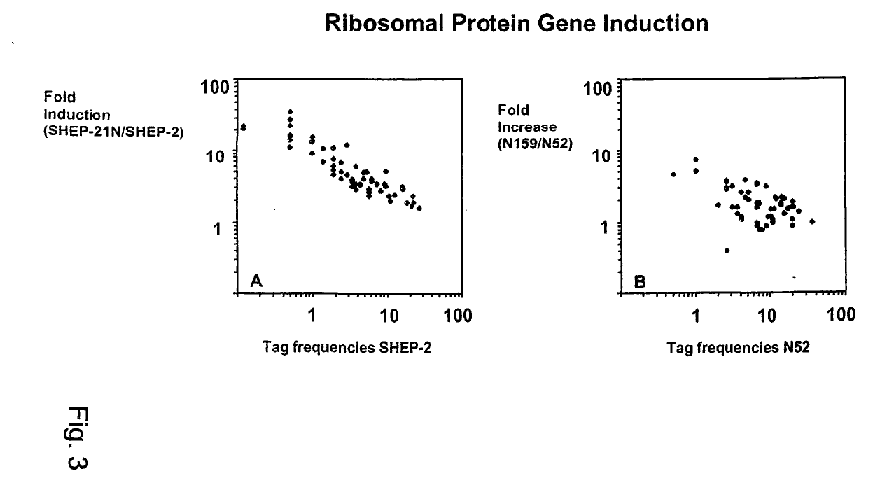

In a time-course experiment, whether the putative N-myc targets are induced after N-myc modulation in the SHEP-21N system was analyzed. N-myc expression can be reversibly switched off in SHEP-21N cells by tetracycline. SHEP-21N cells were treated for 24 hours with tetracycline, washed extensively and grown for an additional 2 to 36 hours without tetracycline. Northern blot analysis showed that the expression of N-myc mRNA is switched off within 8 hours of tetracycline treatment (FIG. 3A, lanes 1-2). After removal of tetracycline, N-myc mRNA expression is restored between 2 and 4 hours (FIG. 3A, lanes 5-6).

The N-myc protein expression was analyzed by Western blotting in a parallel time-course experiment and closely followed the N-myc mRNA expression (FIG. 3B). The Northern blot filters were hybridized with probes for the N-myc downstream targets nucleolin, nucleophosmin and the ribosomal protein genes RPS6 and RPS12 (FIG. 3A). After repression of N-myc by tetracycline, the mRNA levels of these genes remain unaffected at 0 and 8 hours, but their expression was reduced to low basic levels at 24 hours. Importantly, between 2 and 4 hours after re-expression of N-myc mRNA and protein, expression of all four genes was strongly re-induced (FIG. 3B, lanes 6-7).

Similar results were obtained for EEF1A1, TPI1, eIF3 s8, and VDAC (data not shown). The expression level of cofilin that was used as a control does not significantly change during the time course. To exclude a direct effect of tetracycline on nucleolin or nucleophosmin expression, the same experiment was performed with SHEP-2 cells, but no effect on gene expression was observed (data not shown). These results confirm that genes identified herein indeed are induced by N-myc. The results also show that the identified genes are early targets in the N-myc downstream pathway, although not necessarily direct targets of N-myc. Therefore, the identified genes represent essential components of the N-myc pathway. The data further show that the induction by N-myc is highly versatile, as expression drops after N-myc abrogation and is swiftly restored after N-myc re-expression.

N-myc induces ribosomal RNA synthesis.

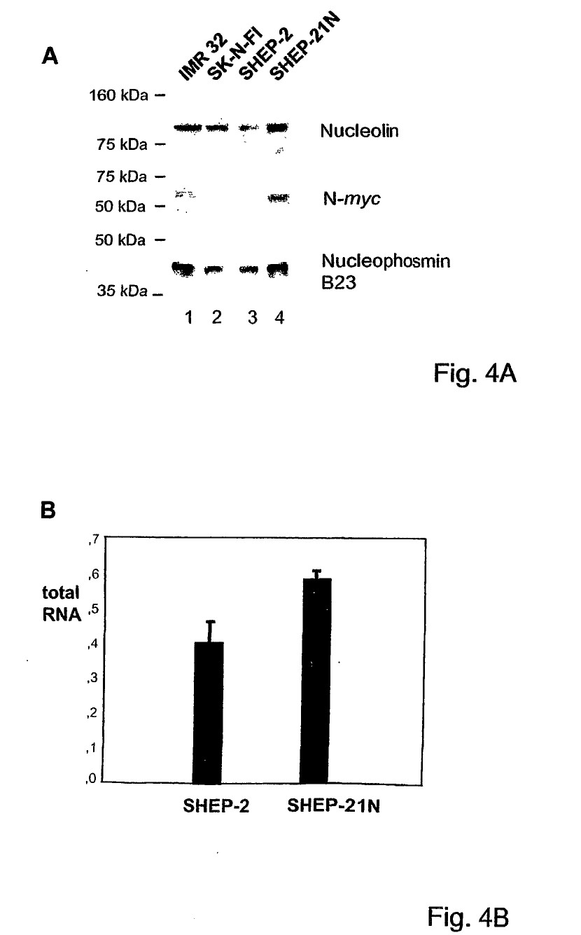

Since the induction of two genes with a key role in rRNA processing and ribosome biogenesis exist, an analysis of their protein expression level and their possible functional activity was performed. Protein expression of nucleolin and nucleophosmin was analyzed in SHEP-2 and SHEP-21N cells, as well as in two control cell lines with and without N-myc amplification. Western blot analyses showed a higher nucleolin and nucleophosmin expression in SHEP-21N compared to SHEP-2 (FIG. 4, lanes 3 and 4) and in the N-myc-amplified IMR32 cell line compared to the N-myc single copy cell line SK-N-FI (FIG. 4, lanes 1 and 2). As these proteins function in ribosomal RNA processing, whether SHEP-21N has a higher rRNA content than SHEP-2 cells was analyzed. Total RNA was isolated from 10 samples of 10⁶ exponentially growing cells of each of the cell lines. Spectrophotometric analysis revealed that SHEP-21N cells have, on average, 45% higher yield of total RNA than SHEP-2 cells (p<0.001, Student T test for independent samples) (FIG. 4B). Duplicate experiments on independently cultured cells gave the same results. Densitometric quantification of the 18S and 24S rRNA bands fractionated by agarose gel electrophoresis confirmed that this increase is caused by ribosomal RNA (data not shown).

To analyze whether this strong increase in rRNA resulted in increased ribosomal function and overall protein synthesis, protein content and the rate of protein synthesis was measured in SHEP-2 and SHEP-21N cells. Lysates of 10⁶ SHEP-2 and SHEP-21N cells contained equivalent amounts of protein (data not shown). Protein synthesis rates were analyzed by ³⁵S-methionine incorporation. No differences were observed between SHEP-2 and N-myc-expressing SHEP-21N cells. Manipulation of the N-myc expression in SHEP-21N in a time course experiment also did not reveal any difference in protein synthesis rates (data not shown). This suggests that the protein synthesis rate in SHEP-21N is either limited by a factor not induced by N-myc, or that protein synthesis is already maximal in the SHEP neuroblastoma cell line and beyond a level that can be boosted by N-myc.

SAGE libraries of neuroblastomas with and without amplification of endogenous N-myc.

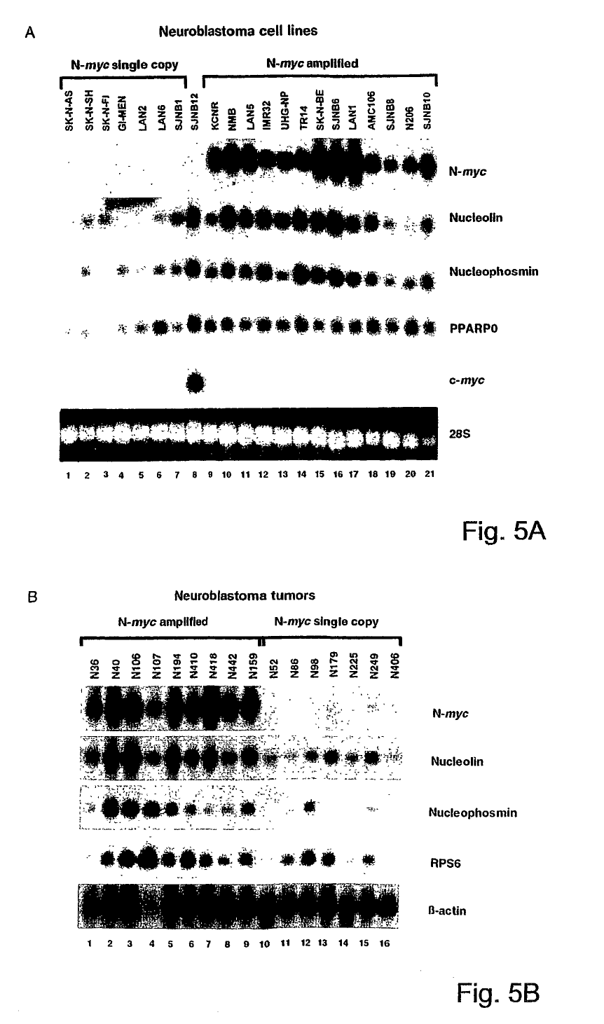

Since the SHEP neuroblastoma cell line has no endogenous N-myc expression, the N-myc-transfected cells do not necessarily have a genetic background representative for N-myc-amplified neuroblastomas. For example, 90% of the N-myc-amplified neuroblastomas have deletions of the chromosomal region 1p35-36 (Caron et al., 1993), while the SHEP-2 and SHEP-21N cells have two apparently intact p arms of chromosome 1 (data not shown). To address the question whether the downstream pathway of N-myc identified herein is also in vivo activated, SAGE libraries of two neuroblastomas were generated. Neuroblastoma tumor N159 has N-myc amplification and expression and neuroblastoma N52 is an N-myc single copy tumor without N-myc expression (FIG. 5B, lanes 9 and 10). 39,598 tags of the two libraries were sequenced. The tag frequencies were normalized per 20,000 tags and compared. N-myc was represented by 16 tags in N159 and 0 tags in N52. There are 52 tags differentially expressed (p<0.01) in the libraries. These differences are probably partly caused by N-myc, as the two tumors are likely to differ in more aspects. The N-myc target genes identified in the SHEP cells that correlate with N-myc in the two tumors were analyzed.

The 56 significantly (p<0.01) induced ribosomal protein genes detected in SHEP-21N produce a total of 988 tags in N52 and 1600 tags in N159 (per 20,000 tags). The N-myc-amplified N159 tumor therefore has a 62% higher ribosomal protein gene expression. There are 36 tags with an increase of at least 50% and 22 tags with an increase of at least 100% in N159 when compared to N52 (FIG. 2B). These increases are more moderate than the SHEP-21N cells (compare FIGS. 2A and 2B), but strongly suggest that N-myc induces ribosomal protein gene expression in vivo.

Other genes functioning in protein synthesis are also up-regulated in N159. Increased expression in N159 compared to N52 is seen for nucleophosmin (from 4 to 19.2 tags), nucleolin (3 to 9 tags), eukaryotic translation initiation factor 4A, isoform 1 (4 to 9 tags), the translation elongation factors EEF1a1 (50 to 96 tags) and EEF1g (18.4 to 32.8 tags). There is almost no induction of the genes involved in glycolysis and oxidative phosphorylation. The expression levels of five representative genes were confirmed by hybridization of Northern blots with total RNA from N159 and N52 (FIG. 5B and data not shown). These results show that the expression levels of many of the N-myc target genes identified in the SHEP-21 N cells are also in vivo correlated with N-myc amplification and overexpression. However, this does not hold for all genes, suggesting that other factors modulate the activity of N-myc target genes.

N-myc target gene expression analyzed in panels of neuroblastoma cell lines and tumors.

To further analyze the induction of N-myc downstream genes in neuroblastoma, the expression of the genes was examined in a panel of neuroblastoma cell lines and tumors. Hybridization of a Northern blot of total RNA from 21 neuroblastoma cell lines showed a fair, albeit imperfect, correlation between expression of N-myc, nucleolin, nucleophosmin and the ribosomal protein PPARP0 (FIG. 5A). Cell line SJNB12 has no N-myc expression, but a very high expression of the N-myc target genes. However, this cell line has c-myc amplification and over-expression (FIG. 5A, lane 7 and Cheng et al., 1995), suggesting that c-myc may induce the same target genes as N-myc (as described herein).

As cell lines are not fully representative of neuroblastoma tumors in vivo, 16 fresh neuroblastomas were analyzed including the aggressive stages 3 and 4 and the less aggressive stages 1, 2 and 4s. A Northern blot analysis showed a fair, overall correlation between expression of N-myc, nucleolin and nucleophosmin (FIG. 5B). There are some exceptions, but the overall results suggest that nucleolin and nucleophosmin are also in vivo targets of N-myc induction. Ribosomal protein S6 (RPS6) expression showed a less consistent relationship with N-myc, indicating that in addition to N-myc, other factors may also modulate its expression.

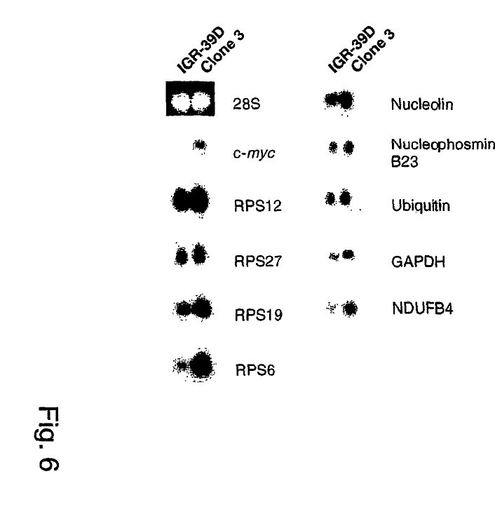

Several N-myc target genes are induced or suppressed by c-myc in addition to N-myc, and belongs to the same family of proto-oncogenes as c-myc. Since both oncogenes induce similar phenotypic effects and share several target genes, whether the N-myc downstream targets identified in this study are targets of c-myc as well were analyzed. Therefore, the melanoma cell line IGR39D) and a c-myc-transfected clone of this cell line (clone 3, Versteeg et al, 1988) were analyzed. Northern blots with total RNA of these cell lines were hybridized with the 26 probes tested on the SHEP-2 and SHEP-21N cells. 9 of 23 N-myc-induced targets appeared to be induced by c-myc as well (FIG. 6) and include the ribosomal protein genes S12, S27, S 19, S6, nucleolin, nucleophosmin, ubiquitin, GAPDH and NDUFB4. Three of the N-myc-suppressed targets were tested and found to be suppressed by c-myc as well and include Osteonectin (Table 1, Nos. 118, 123 and 125), Plasminogen activator inhibitor type 1 (Table 1, No. 112) and connective tissue growth factor (Table 1, No. 127). Therefore, c-myc and N-myc share about 46% of their target genes in the cell systems tested herein including nucleophosmin, nucleolin and most ribosomal protein genes.

86 transcripts were found to contribute to ribosome biogenesis, mRNA translation, protein maturation and protein turnover, demonstrating that enhancement of protein synthesis is a major function of N-myc. A striking 45% higher rRNA content was found in SHEP-21N than in SHEP-2. No overall increase was observed in the rate of protein synthesis in SHEP-21N. One interpretation is that some rate limiting components of the protein synthesis machinery are not induced in SHEP-21N cells. The SAGE libraries of the N-myc single copy neuroblastoma N52 and the N-myc-amplified tumor N159 showed that the ribosomal protein genes, nucleolin, nucleophosmin and five translation initiation and elongation factors are over-expressed in the N-myc-amplified neuroblastoma in vivo. The Northern blot analysis of 37 neuroblastomas and neuroblastoma cell lines further confirmed induction of these genes in N-myc-amplified neuroblastoma. These results show that myc genes function as major regulators of protein synthesis which is in line with the reduced rate of protein synthesis in fibroblasts with a homozygous inactivation of c-myc (Mateyak et al., 1997) and the increased protein synthesis in fibroblast after activation of c-myc (Schmidt, 1999).

Energy production, mitochondria and apoptosis.

Other comprehensive sets of N-myc downstream target genes are implicated in the glycolysis, the mitochondrial electron transfer and ATP synthesis pathways. The identification of the electron transfer and ATP synthesis pathway as a major target of N-myc induction bears on the relationship between the mitochondrial transmembrane potential and apoptosis. Mitochondria have two faces as they provide the energy for fast cycling cells and can drive the cell into apoptosis. Similarly, the myc oncogenes can induce vigorous cell proliferation as well as massive apoptosis. N-myc expression renders SHEP-21N cells susceptible to apoptotic triggers (Fulda et al., 1999; Lutz et al., 1998). Many key events in apoptosis focus on mitochondrial membrane potential (Green and Reed, 1998). Examples are cytochrome c release, hyperpolarization of the inner membrane, opening of the permeability transition pore and generation of reactive oxygen species (ROS). During normal electron transport in the mitochondrial membrane, 1 to 5% of the electrons lose their way and generate ROS. Any interruption of the electron transfer pathway strongly increases ROS production with a deleterious effect on the cell (Kroemer et al., 1997).

Enhancement of the electron flow by N-myc would, upon interruption of the electron transfer chain, boost ROS production. In addition, the moderate up-regulation of VDAC (FIG. 1) could stimulate cytochrome c release and apoptosis. Therefore, N-myc induction of the electron transfer genes provides the energy required for cell proliferation. Meanwhile, it could increase the deadly potential of the mitochondria and upon triggering, tip the scale towards execution of apoptosis.

Tags for oxidative phosphorylation pathways are not over-expressed in the N-myc-amplified N159 tumor. This tumor might have been selected in vivo for additional defects that interfere with part of the N-myc downstream pathway. While SHEP-21N cells expressing N-myc are susceptible to apoptotic triggers (Lutz et al., 1998), neuroblastoma cell lines with overexpression of endogenous N-myc are refractory to such triggers. This shows that these cell lines have defects in the pro-apoptotic arm of the N-myc downstream pathway.

N-myc and c-myc share target genes.

To date, only two target genes of N-myc have been published and are targets of c-myc as well (Lutz et al., 1996; Eilers et al., 1991; Bello-Fernadez et al., 1993). Of the 23 up-regulated targets of N-myc that were tested on Northern blots, 9 are induced by c-myc in transfected melanoma cells. Both down-regulated N-myc targets that were tested were also down-regulated by c-myc. Since the N-myc-induced downstream pathway genes form very concise functional groups of genes, it was postulated that N-myc may function as a general stimulator of protein synthesis and energy production. Since c-myc has an equally powerful growth-inducing and transforming effect as N-myc, it is difficult to envisage that c-myc would only induce a subset of the genes that are necessary to boost the protein and ATP synthesis machineries. It appears that N-myc and c-myc activate the same basic cellular functions. Indeed, c-myc is implicated in induction of protein synthesis in fibroblast cell lines (as described herein). Induction of genes by N-myc strongly depends on their basic expression levels (FIG. 2). It is therefore possible that high expression of potential target genes in the original melanoma cell line may have prevented their induction by c-myc.

The physiological role of myc genes has been enigmatic, as only very few target genes have been identified thus far. 351 transcript tags are described that identify 335 genes defined by their unigene number that are targets of N-myc or potential targets of N-myc, some of which are targets of c-myc as well. Myc genes thus function as major regulators of protein synthesis and cellular energy production and it is likely that this induction mediates the enhanced transition through the G1 phase of the cell cycle in normally proliferating cells and in cells that are induced to proliferate by physiological stimuli. The effect on protein synthesis confirms earlier postulations based on the identification of a limited set of target genes (Schmidt, 1999; Mateyak et al., 1997; Johnston et al., 1999). The stimulatory effect on genes in the electron transfer and ATP synthesis pathway is unexpected and fits well with the energy requirements for enhanced protein production, GI transition and could also relate to the apoptotic effect of myc genes.

List of tags and genes induced or suppressed by N-myc.

Table 1 lists the tags that were found to be significantly (p<0.01) induced or suppressed by N-myc in the comparison of the SHEP-2 and SHEP-21N SAGE libraries. Table 1A is disclosed to designate the nucleotide sequences of at least some of the tags of Table 1. The comparison is based on 21,559 tags of SHEP-21N and 44,674 tags of SHEP-2. The tag frequencies shown are normalized per 10,000 tags (column SHEP-2 and SHEP-21N). The column “ratio ON:OFF” shows the fold induction (positive values) or suppression (negative values) by N-myc. When a tag had a zero expression in one of the libraries, it was assumed for ratio calculation that the tag was present one time in the entire library. The Unigene numbers of the National Center for Biotechnology Information (NCBI, Bethesda, USA) are given in the column “Unigene.” The numbers are based on the NCBI Unigene database as by 29-3-2000. The next column shows the Unigene description. Furthermore, for each Unigene cluster, one or two Genbank accession codes are given. For some tags, two possible corresponding genes were identified which is indicated by an asterisk in the column next to the tag.

Table 2 lists tags that were identified to differ significantly (p<0.01) between the SAGE libraries of SHEP-2 and SHEP-21N after extending the sequencing of library SHEP-21N from 21,559 tags to 34,426 tags. Table 2 lists expression levels in both libraries expressed per 20,000 transcript tags (column “SHEP-2” and “SHEP-21N”), the unigene number as identified by the computer program described by Caron et al. (2001) and in some cases a Genbank accession number of a clone corresponding to the Unigene cluster.

Experimental Procedures.

Cell Lines.

Neuroblastoma cell lines and culture conditions were as described (Cheng et al., 1995). The melanoma cell lines IGR39D and clone 3 were described by Versteeg et al. (1988). The SHEP cell lines were maintained in RPMI 1640 medium supplemented with 10% fetal calf serum, 4 mM L-glutamine, 100 U/ml penicillin and 100 μg/ml streptomycin (Lutz et al., 1996). Tetracycline (Sigma) was used at a concentration of 10 ng/ml medium to inhibit N-myc expression.

Generation of SAGE Libraries.

SAGE was performed as described by Velculescu et al. (1995) with a few adaptations. Total RNA was extracted by guanidium thiocyanate (Chromczynski and Sacchi, 1987). Poly(A)⁺ RNA was isolated using the MessageMaker kit (Gibco/BRL) according to the manufacturer's instructions. SAGE libraries were generated using minimally 4 μg poly(A)⁺ RNA. The cDNA was synthesized according to the Superscript Choice System (Gibco/BRL), digested with NlaIII and bound to streptavidin-coated magnetic beads (Dynal). Linkers containing recognition sites for BsmFI were ligated to the cDNA. Linker tags including a cDNA tag were released by BsmFI digestion, ligated to one another and amplified. The PCR products were heated for 5 minutes at 65° C. before preparative analysis on a polyacrylamide gel. After the ligation into concatameres, a second heating step was included (15 minutes at 65° C.) and fragments between 800 bp and 1500 bp were purified and cloned in pZero-1 (Invitrogen). Colonies were screened with PCR using M13 forward and reverse primers. Inserts larger than 300 bp were sequenced with a BigDye terminator kit and analyzed on a 377 ABI automated sequencer (Perkin Elmer).

Analysis SAGE Database.

The SAGE libraries were analyzed using the SAGE 300 program software package (Velsculescu et al., 1997). P-values were calculated using Monte Carlo simulations. Transcripts were identified by comparison of the tags in the database with the “tag to gene map” (SAGEmap) from Cancer Genome Anatomy Project available from NCBI. This database links Unigene clusters to SAGE tags (Lal et al., 1999). The gene assignments were subsequently checked by hand or sequencing errors causing incorrect tags and for erroneous gene assignments based on hybrid Unigene clusters. Other database analysis and generation of specific primers utilized the Wisconsin GCG package software.

Northern Blot Analysis.

Total RNA (20 μg per lane) was electrophoresed through a 0.8% agarose gel in the presence of 6.7% formaldehyde and blotted on Hybond N membranes (Amersham) in 10×SSC. Hybridization was carried out overnight in 0.5 M NaHPO₄, pH 7.0, 7% SOS, 1 mM EDTA at 65° C. Filters were washed in 40 mM NaHPO₄, 1% SDS at 65° C. Probes were labelled by random priming of sequence-verified PCR products.

Total Protein Content.

Exponentially growing cells were harvested and the number of cells was determined using a Coulter counter. Cells (1×10⁶) were lysed in 20 mM Tris-HCl (pH 8.0), 137 mM NaCl, 10% glycerol, 1% NP40 and protease inhibitors (protease cocktail, Roche). Samples were assayed with the Bio-Rad Protein assay. Assays were performed at least in duplicate.

Western Blots.

Cell lysates were separated on SDS-polyacrylamide gel and electroblotted onto Immobilon-P transfer membrane (Millipore). Blocking of the membrane and incubation with antibodies involved standard procedures. Proteins were visualized using the ECL detection system (Amersham). Anti-nucleophosmin monoclonal antibody was a gift of Dr. P. K. Chan (Baylor College of Medicine). The antibody against nucleolin was a gift of Dr. P. Bouvet (CNRS, IPBS, Toulouse, France). Anti-N-myc was obtained from PharmIngen (Clone B8.4.B).

Total rRNA Content.

Total RNA of 1×10⁶ exponentially growing cells was extracted by guanidium isothiocyanate (Chromczynski and Sacchi, 1987) and photospectrometrically quantified. Results of ten isolations of each of the cell lines SHEP-2 and SHEP-21N were statistically analyzed with the Students T test for independent samples. Aliquots on a per cell basis were subjected to agarose gel electrophoresis and stained with ethidium bromide. The relative fluorescence of the rRNA bands was quantified using the Kodak Digital Science 10 Image Analysis Software package (EDAS 120).

### REFERENCES

Alex, R., Sozeri, O., Meyer, S., Dildrop, R. (1992). Determination of the DNA sequence recognized by the bHLH-zip domain of the N-Myc protein. Nucleic Acids Res. 20,2257-2263.

Amati, B., Alevizopolous, K., Vlach. (1998). Myc and the cell cycle. Front. Biosci 3, D250-D268.

Bello-Femandez, C., Packham, G., Cleveland, J. L. (1993). The ornithine decarboxylase gene is a transcriptional target of c-Myc. Proc. Natl. Acad. Sci. USA 90, 7804-7808.

Benvenisty, N., Leder, A, Kuo, A., Leder, P. (1992). An embryonically expressed gene is a target for c-Myc regulation via the c-Myc-bincling sequence. Genes Dev. 6, 2513-2523.

Bernards, R., Dessain, S. K., Weinberg, R. A. (1986). N-myc amplification causes down-modulation of MHC class I antigen expression in neuroblastoma. Cell 47, 667-674.

Blackwood, E. M., Kretzner, L., Eisenman, R. N. (1992). Myc and Max function as nucleoprotein complex. Curr. Opn. Genet. Dev. 2, 227-235.

Boon, K., Caron, H. N., van Asperen, R., Valentijn, L., Hermus, M. C., van Sluis, P., Roobeek, I., Weis, I., Voute, P. A., Schwab, M., Versteeg, R. (2001). N-myc enhances the expression of a large set of genes functioning in ribosome biogenesis and protein synthesis. EMBO J. Mar. 15, 2001;20(6):1383-93.

Caron, H., van Schaik, B., van der Mee, M., Baas, F., Riggins, G., van Sluis, P., Elermus, M. C., van Asperen, R., Boon, K., Voute, P. A., Heisterkamp, S., van Kampen, A., Versteeg, R. (2001). The human transcriptome map: clustering of highly expressed genes in chromosomal domains. Science, Feb. 16, 2001; 291(5507):1289-92.

Borer, R. A., Lehner, C. F., Eppenberger, H. M., Nigg, E. A. (1989). Major nucleolar proteins shuttle between nucleus and cytoplasma. Cell 56, 379-390.

Bouvet, P., Diaz, J. J., Kindbeiter, K., Madjar, J. J., Amalric., F. (1998). Nucleolin interacts with several ribosomal proteins through its RGG domain. Journal of Biol. Chem. 278,19025-19029.

Boyd, K. E., Farnham, P. J. (1997). Myc versus USF: discrimination at the cad gene is determined by core protein elements. Mol. Cell. Biol. 17, 2529-2537.

Caron, H., van Sluis, P., van Hoeve, M., de Kraker, J., Bras, J., Slater, R., Mannes, M., Voute, P. A., Westerveld, A., Versteeg, R. (1993). Allelic loss of chromosome 1p36 in neuroblastoma is of preferential maternal origin and correlates with N-myc amplification. Nature Genetics 4, 187-190.

Chan, P. K., Aldrich, M. B., Yung, B. Y. M. (1987). Nucleolar protein B23 translocation after doxorubicin treatment in murine tumor cells. Cancer Res. 47, 3798-3801.

Chan, P. K., Aldrich, M., Chakrabarty, S. (1988). Assessment of tumor cell sensitivity to mitomycin C by B23 translocation assay. Cancer Lett. 40, 143-49.

Chan, P. K. Characterization and cellular localization of nucleophosmin/B23 in HELA cells treated with cytostatic agents. Exp. Cell Res. 203, 174-181.

Chan, P. K., Chan, F. Y., Morris, S. W., Xie Z. (1997). Isolation and characterization of the human nucleophosmin B23 (NPM) gene: identification of the YYI binding site at the 5′ enhancer region. Nucleic Acids Res 25, 1225-1232.

Cheng, N. C., Van Roy, N., Chan, A., Beitsma, M., Westerveld, A., Speleman, F., Versteeg, R. (1995). Deletion mapping in neuroblastoma cell lines show two distinct tumor suppressor genes in the 1p35-36 region, only one of which is associated with N-myc amplification. Oncogene 10, 291-297.

Chomczynski, P., Sacchi, N. (1987). Single-step method of RNA isolation by acid guanidiniumthiocyanate-phenol-chloroform extraction. Anal Biochem 162, 156-159.

Chung, S., Perry, R. P. (1993). The importance of downstream delta-factor binding elements for the activity of the rpL32 promoter. Nucleic Acids Research 21, 3301-3308.

Cohen, M. B., Glazer, R. I. (1985). Comparison of the cellular and RNA-dependent effects of sangivamycin and toyocamycin in human colon carcinoma cells. Mol Pharmacol 27, 349-55.

Cole, M. D. (1986). The myc oncogene: its role in transformation and differentiation. Annu Rev Genet. 20, 361-84.

Cole, M. D., McMahon, S. B. (1999). The Myc oncoprotein: a critical evaluation of transactivation and target gene regulation. Oncogene 18, 2916-2924.

Cooper, H. L., Gibson, E. M. (1971). Control of synthesis and wastage of ribosomal ribonucleic acid in lymphocytes II. The role of protein synthesis. J. Biol. Chem. 246, 5059-5066.

Daskis, J. I., Lu, R. Y., Facchini, L. M., Marhin, W. W., Penn, L. J. Z. Myc induces cyclin D1 expression in the absence of the novo protein synthesis and links mitogen-stimulated signal transduction to the cell cycle. (1994). Oncogene 9, 3635-3645.

Dang, C. V. and Lee, L. A. (1995). C-Myc function in neoplasia. R. G. Landes and Springer Verlag, Austin Tex.

Dang, C. V. (1999). C-Myc target genes involved in cell growth, apoptosis and metabolism. Mol. Cell. Biol. 19, 1-11.

Dudov, K. P., Dabeva M D. (1983). Post-transcriptional regulation of ribosome formation in the nucleus of regenerating rat liver. Biochem J. 210, 183-192.

Eilers, M., Schrim, S., Bishop, J. M. (1991). The myc protein activates transcription of the alpha-prothomyosin gene. EMBO Journal 10, 133-141.

Evan, G. I., Wyllie, A. H., Gilbert, C. S., Littlewood, T. H., Land, H., Brooks, M., Waters, C. M., Penn, L. Z., Hancock, D. C. (1992). Induction of apoptosis in fibroblasts by c-myc protein. Cell 69, 119-128.

Fulda, S., Lutz, W., Schwab, M., Debatin, K. M. (1999). Myc-N sensitizes neuroblastoma cells for drug-induced apoptosis. Oncogene 18, 1479-1486.

Galaktionov, K., Chen, X., Beach, D. (1996). Cdc25 cell cycle phosphatase as a target of c-myc. Nature 382, 511-517.

Gamberi, G., Benassi, M. S., Bohling, T., Ragazzini, P., Molendini, L., Solazzo, M. R., Merli, M., Ferrari, C., Magagnoli, G., Bertoni, F., Picci, P. (1998). Prognostic relevance of c-myc gene expression in giant-cell tumor of bone. J. of Orthopaedic Res. 16, 1-7.

Ginisty, H., Almalric, F., Bouvet, P. (1998). Nucleolin functions in the first step of ribosomal RNA processing. EMBO Journal 17, 1476-1486.

Ginisty, H., Sicard, H., Roger, B., Bouvet, P. (1999). Structure and function of nucleolin. J. Cell Sci. 112, 761-772.

Grandori, C., Mac, J., Siebelt, F., Ayer, D. E., Eisenman, R. N. (1996). Muyc-Max heterodimers activate a DEAD box gene and interact with multiple E box-related sites in vivo. EMBO Journal 15, 4344-4357.

Greasley, P. J., Bonnard, C., Amati, B. (2000). Myc induces the nucleolin and BN51 genes: possible implications in ribosome biogenesis. Nucleic Acid Research 28, 446-453.

Green, D., Reed, J. C. (1998). Mitochondria and apoptosis. Science 281, 1309-1312.

Hanson, K. D., Schichiri, M., Follansbee, M. R., Sedivy, J. M. (1994). Effects of c-myc expression on cell cycle progression. Mol. Cell. Biol. 14, 5748-5755.

Hariharan, N., Kelley, D. E., Perry, R. P. (1991). Delta, a transcription factor that binds to downstream elements in several polymerase II promoters, is a functionally versatile zinc finger protein. Proc. Natl. Acad. Sciences 88, 9799-9803.

Henriksson, M., and Luscher, B. (1996). Proteins of the Myc network: essential regulators of cell growth and differentiation. Adv. Cancer Res. 68, 109-182.

Jansen-Durr, P., Meiche, A., Steiner, P., Pagano, M., Finke, K., Botz, J., Wessbecher, J., Draetta, G., Eilers, M. (1993). Differential modulation of cyclin gene expression by Myc. Proc. Natl. Acad. Sci. USA 90, 3685-3689.

Johnston, L. A., Prober, D. A., Edgar, B. A., Eiseman, R N., Gallant, P. (1999). Drosophila myc regulates cellular growth during deveopment. Cell 98, 779-790.

Jones, R. M., Branda, J., Johnston, M., Polymensis, Gadd, M., Rustgi, A., Callanan, L., Schmidt, E. V. (1996). An essential E box in the promoter of the gene encoding the mRNA cap-binding protein (eukaryotic initiation factor 4E) is a target for activation by c-myc. Mol. Cell. Biol. 16, 4754-4764.

Kakisako, K., Miyahara, M., Uchino, S., Adachi, Y., Kitano, S. (1998). Prognostic significance of c-myc mRNA expression assessed by semi-quantitative RT-PCR in patients with colorectal cancer. Oncology Reports 5, 441-5.

Komarov, P. G., Komarova, E. A., Kondratov, R. V., Christov-Tselkov, K., Coon, J. S., Chernov, M. V. and Gudkov, A. V. (1999). Science 285, 1733-37.

Kroemer, G., Zamzami, N. and Susin, S. A. (1997). Mitochondrial control of apoptosis. Immun. Today 18, 45-51.

Lane, T. F. and Sage, E. H. (1994). The biology of SPARC, a protein that modulates cell-matrix interactions. FASEB J. 8, 163-173.

Lal A., Lash, A. E., Altschul, S. F., Velculescu, V., Zhang, L., McLendon, R. E., Marra, M. A., Prange, C., Morin, P. J., Polyak, K., Papadopoulos, N., Vogelstein, B., Kinzler, K. W., Strausberg, R. L., Riggins, G. J. (1999). A public database for gene expression in human cancers. Cancer Res 59, 5403-7.

Lutz, W., Stöhr, M., Schürmann, J., Wenzel, A., Löhr, A., Schwab, M. (1996). Conditional expression of N-myc in human neuroblastoma cells increases expression of α-prothymosin and ornithine decarboxylase and accelerates progression into S-phase early after mitogenic stimulation of quiescent cells. Oncogene 13, 803-812.

Lutz, W., Fulda, S., Jeremias, I., Debatin, K. M., Schwab, M. (1998). MycN and IFN gamma cooperate in apoptosis of human neuroblastoma cells. Oncogene 17, 339-346.

Ma, A., Mercy T., Collum, R., Weintraub, H., Alt, F. W., Blackwell, T. K. (1993). DNA binding by N- and L-Myc proteins. Oncogene 8, 1093-1098.

Marcu, K. B., Bossone, S. A., Patel, A. J. (1992). myc function and regulation. Annu Rev Biochem 61, 809-60.

Marx, N., Bourcier, T., Sukhova, G. K., Libby, P., Plutzky, J. (1999). PPARgamma activation in human endothelial cells increases plasminogen activator inhibitor type-1 expression: PPARgamma as a potential mediator in vascular disease. Arterioscler Thromb Vasc Biol. 19, 546-51.

Mateyak, M. K., Obaya, A. J., Adachi, S., Sedivy, J. M. (1997). Phenotypes of c-Myc-deficient rat fibroblasts isolated by targeted homologous recombination. Cell Growth Differ. 8, 1039-1048.

Morris, S. W., Kirstein, M. N., Valentine, M. B., Dittmer, K. G., Shapiro, D. N., Saltman, D. L., Look, A. T. (1994). Fusion of a kinase gene, ALK, to a nucleolar protein gene, NPM, in non-Hodgkin's lymphoma. Science 263, 1281-1284.

Nicoloso, M., Caizergues-Ferrer, M., Michot, B., Azum, M. C., Bachellerie, J. P. (1994). U20, a novel small nucleolar RNA, is encoded in an intron of the nucleolin gene in mammals. Mol. Cell Biol 14, 5766-5776.

Nicoloso, M., Qu, L. H., Michot, B., Bachellerie, J. P. (1996). Intron-encoded, antisense small nucleolar RNAs: the characterization of nine novel species points to their direct role as guides for the 2′-O-ribose methylation of rRNAs.

Nisen, P. D., Zimmerman, K. A., Cotter, S. Y., Gilbert, F., Alt, F. W. (1986). Enhanced expression of the N-myc gene in Wilms' tumors. Cancer Res. 46, 6217-22.

Olson, M. O. (1991). The role of protein in nucleolar structure and function in The Eukaryotic Nucleus: Molecular Biochemistry and Macromolecular Assemblies (Strauss, P. R., Wilson, S. H. eds.) Vol. 2, 541-546. Telford Press, Caldwell, N. J.

Pandolfi, P. P. (1996). PML, PLZF and NPM genes in the molecular pathogenesis of acute promyelocytic leukemia. Haematologica 5, 472-482.

Perlaky, L., Valdez, B. C., Busch, H. (1997). Effects of cytotoxic drugs on translocation of nucleolar RNA helicase RH-II/Gu. Exp. Cell Research 235, 413-20.

Peter, M., Nakagawa, J., Doree, M., Labbe, J. C., Nigg, E. A. (1990). Identification of major nucleolar proteins as candidate mitotic substrates of cdc2 kinase. Cell 60, 791-801.

Philipp, A., Schneider, A., Varsik, I., Finke, K., Xiong, Y., Beach, D., Alito, K., Eilers, M. (1994). Repression of cyclin D1: a novel function of Myc. Mol. Cell. Biol. 14, 4032-4043.

Polyak, K., Xia, Y, Zweier, J. L., Kinzler, K. and Vogelstein, B. (1997). A model for p53-induced apoptosis. Nature 389, 300-305.

Qu, L. H., Nicoloso, M., Michot, B., Azum, M. C., Caizergues-Ferrer, M., Renalier, M. H., Bachellerie, J. P. (1994). U21, a novel small nucleolar RNA with a 13 nt. Complementarity to 288 rRNA, is encoded in an intron of ribosomal protein L5 gene in chicken and mammals. Nucleic Acid Research 22, 4073-4081.

Redner, R. L., Rush, E. A., Faas, S., Rudert, W. A., Corey, S. J. (1996). The t(5;17) variant of acute promyelocytic leukemia expresses a nucleophosmin-retinoic acid receptor fusion. Blood 87, 882-886.

Rosenwald, I. B., Rhoads, D. B., Callanan, L. D., Isselbacher, K. J., Schmidt, E. V. (1993). Increased expression of eukaryotic translation initiation factors eIF-4E and eIF-2 alpha in response to growth induction by c-myc. Pro. Natl. Acad. Sci. USA 90, 6175-6178.

Savkur, R. S., Olson, M. O. J. (1998). Preferential cleavage in pre-ribosomal RNA by protein B23 endonuelease. Nucleic Acid Research 26, 4508-4515.

Seeger, R. C., Brodeur, G. M., Sather, H., Dalton, A., Siegel, S. E., Wong, K. Y., Hammond, D. (1985). Association of multiple copies of the N-myc oncogene with rapid progression of neuroblastomas. N. Engl. J. Med. 318, 1111-1116.

Schwab, M., Alitalo, K., Klempnauer, K. H., Varmus, H. E., Bishop, J. M., Gilbert, F., Brodeur, G. M., Goldstein, M., Trent, J. M. (1983). Amplified DNA with limited homology to myc cellular oncogene is shared by human neuroblastoma cell lines and a neuroblastoma tumour. Nature 305, 245-248.

Shimizu, S., Narita, M., Tsujimoto, Y. (1999). Bcl-2 family proteins regulate the release of apoptogenic cytochrome c by the mitochondrial channel VDAC. Nature 399, 483-487.

Schlagbauer-Wadl, H., Griffioen, M., van Elsas, A., Schrier, P. T., Pustelnik, T., Eichler, H. G., Wolff, K., Pehamberger, H., Jansen, B. (1999). Influence of increased c-myc expression on the growth characteristics of human melanoma. J Invest Dermatol 112, 332-6.

Schmidt, E. V. (1999). The role of c-myc in cellular growth. Oncogene 18, 2988-2996).

Solomon, D. L. C., Philipp, A., Land, H., Eilers, M. (1995). Expression of cyclin D1 mRNA is not up-regulated by Myc in Rat fibroblasts. Oncogene 11, 1893-1897.

Sierra, A., Castelsague, X., Escobedo, A., Moreno, A., Drudis, T., Fabra, A. (1999). Synergistic cooperation between c-myc and Bcl-2 in lymph node progression of Ti human breast carcinomas. Breast Cancer Res. Treat. 54, 39-45.

Steiner, P., Philipp, A., Lukas, J., Godden-Kent, P., Pagano, M., Mittnacht, S., Bartek, J., Eilers, M. (1995). Identification of a Myc-dependent step during the formation of active GI cyclin-cdk complexes. EMBO Journal 14, 4814-4826.

Strojan, P., Budihna, M., Smid, L., Skrk, J. (1998). Urokinase-type plasminogen activator (uPA) and plasminogen activator inhibitor type 1 (PAI-1) in tissue and serum of head and neck squamous cell carcinoma patients. Eur J Cancer 34, 1193-7.

Szebeni, A., Olson, M. O. (1999). Nucleolar protein B23 has molecular chaperone activities. Protein Sci 8, 905-912.

Tujeta, R., Tujeta, N. (1998). Nucleolin: a multifunctional major nucleolar phosphoprotein. Crit. Rev Biochem Mol Biol 33, 407-436.

Velculescu, V. E., Zhang, L., Vogelstein, B., and Kinzler, K. W. (1995). Serial analysis of gene expression. Science 270, 484-487.

Velculescu, V. E., Zhang, L., Zhou, W., Volgelstein, J., Basrai, M. A., Basset Jr., 20 D. E., Hieter, P., Volgelstein, B., and Kinzler, K. W. (1997). Characterization of the yeast transcriptome. Cell 88, 243-251.

Versteeg, R., Noordermeer, I. A., Krilse-Wolters, M., Ruiter, D, J., and Schrier, P.1. (1988). C-myc down-regulates class I HLA expression in human melanomas. EMBO J. 7, 1023-1029.

Weiss, W. A., Aldape, K., Mohapatra, G., Feuerstein, B. G., Bishop, J. M. (1997). Targeted expression of MYCN causes neuroblastoma in tranagenic mice. EMBO Journal 16, 2985-2995.

Welle, S., Bhatt, K., Thornton, C A. (1999). Inventory of High-Abundance mRNAs in Skeletal Muscle of Normlal Men. Genome Research 9, 506-513.

Wiedmann, B., Sakai, H., Davis, T. A., Wiedmann, M. (1994). A proteincomplex required for signal-sequence-specific sorting and translocation. Nature 370, 434-440.

Wilson, W. (1968). Phase I study with toyocamycin (NSC-63701). Cancer Chemotherapy Reports 52, 301-3.

Wool, I., Chan, Y. L., Glück, A., (1996). In Translational Control, (Hershey, J., Mathews, M., and Sonenberg, N., eds), 685-732, Cold Spring Harbor Laboratory Press, Cold Spring Harbor, N.Y.

Yung, B. Y. M., Busch, H., Chan, P. K. (1985). Translocation of nucleolar phosphoprotein B23 (37 kDa/pI 5.1) induced by selective inhibitors of ribosome synthesis. Biochim. Biophys. Acta. 826, 167-173.

Yung, B. Y. M., Bor, A. M. S., Chan, P. K. (1990). Short exposure to actinomycin D induces reversible translocation of protein B23 as well as reversible inhibition of cell growth and RNA synthesis in HeLa cells. Cancer Res. 50, 5987-91.

TABLE 1
MYCN regulated genes (grouped by functional category)

| | | | | | | | | RIBOSOMAL PROTEINS | |
|---|---|---|---|---|---|---|---|---|---|
| [symbol] | [symbol] | | 47.7 | 0.000 | 0.0 | 10.7 | 5174 | ribosomal protein S17 | AA876041, AI564812, |
| [symbol] | [symbol] | | 43.5 | 0.000 | 0.0 | 9.7 | 180920 | ribosomal protein S9 | AI064904, U14971 |
| [symbol] | [symbol] | | 38.3 | 0.000 | 0.4 | 17.2 | 82148 | ribosomal protein S12 | AA483128, AA524764, |
| [symbol] | [symbol] | | 29.0 | 0.000 | 0.2 | 6.5 | 75458 | ribosomal protein L18 | L11566, AA513721, |
| [symbol] | [symbol] | | 26.9 | 0.000 | 0.2 | 6.0 | 178391 | L44- [symbol] ribosomal protein (L44L), Homo sapiens Brutants tyrosine kinase (BTK), alpha-D-gal | W05120, AA181201, |
| [symbol] | [symbol] | | 20.7 | 0.000 | 0.7 | 13.9 | 8102 | ribosomal protein S20 | AI741190, AA064902, |
| [symbol] | [symbol] | | 16.6 | 0.000 | 0.4 | 7.4 | 165590 | ribosomal protein S13 | AI350382, AI290903, |
| [symbol] | [symbol] | | 16.6 | 0.000 | 0.9 | 14.8 | 80617 | ribosomal protein S16 | AI262558, AA628078, |
| [symbol] | [symbol] | | 12.4 | 0.006 | 0.2 | 2.8 | 3254 | ribosomal protein L23-like | U26596, Z49254, |
| [symbol] | [symbol] | * | 12.4 | 0.006 | 0.2 | 2.8 | 1948 | ribosomal protein S21 | AW196629 |
| [symbol] | [symbol] | | 12.4 | 0.000 | 0.7 | 8.3 | 179666 | ribosomal protein L35a | AW328435, AW328458, |
| [symbol] | [symbol] | | 11.4 | 0.000 | 0.4 | 5.1 | 74267 | ribosomal protein L15 | AW327407, AA689521, |
| [symbol] | [symbol] | | 11.4 | 0.000 | 1.3 | 15.3 | 73742 | ribosomal protein, large, P0 | AA807754, AI174682, |
| [symbol] | [symbol] | | 11.0 | 0.000 | 3.1 | 34.3 | 182426 | ribosomal protein S2 | X17206, AA704039, |
| [symbol] | [symbol] | | 9.9 | 0.000 | 2.2 | 22.3 | 178551 | ribosomal protein L8 | AW327732, AI200056, |
| [symbol] | [symbol] | | 9.2 | 0.000 | 1.6 | 14.4 | 153177 | ribosomal protein S28 | AA229774, AA421061, |
| [symbol] | [symbol] | * | 8.3 | 0.010 | 0.0 | 1.9 | 128400 | ESTs, Highly similar to 60S RIBOSOMAL PROTEIN L39 [H. sapiens] | AA075869 |
| [symbol] | [symbol] | | 8.0 | 0.000 | 1.6 | 12.5 | 180450 | ribosomal protein S24 | AW410149, D51704, |
| [symbol] | [symbol] | | 7.7 | 0.000 | 2.2 | 17.2 | 75362 | ribosomal protein S18 | AA178543, AW409851, |
| [symbol] | [symbol] | | 7.4 | 0.000 | 1.6 | 11.6 | 2017 | ribosomal protein L38 | AA659304, AA747113, |
| [symbol] | [symbol] | | 7.1 | 0.000 | 1.6 | 11.1 | 91379 | ribosomal protein L26 | AA084377, AA111974, |
| [symbol] | [symbol] | | 6.6 | 0.000 | 1.1 | 7.4 | 99914 | ribosomal protein L22 | AA420829, AA603500, |
| [symbol] | [symbol] | | 6.0 | 0.000 | 1.8 | 10.7 | 157850 | ribosomal protein L9 | D14531, U09953, |
| [symbol] | [symbol] | | 5.9 | 0.000 | 3.1 | 18.6 | 182825 | ribosomal protein L35 | AA457581, AA479946, |
| [symbol] | [symbol] | | 5.9 | 0.000 | 2.7 | 15.8 | 119598 | ribosomal protein L3 | AA121930, AW022183, |
| [symbol] | [symbol] | | 5.7 | 0.002 | 0.9 | 5.1 | 158675 | ribosomal protein L14 | AA857488, AA186408, |
| [symbol] | [symbol] | | 5.7 | 0.000 | 8.3 | 46.8 | 163698 | ribosomal protein L29 | U10248, U49083, |
| [symbol] | [symbol] | | 5.3 | 0.000 | 4.9 | 26.0 | 163593 | ribosomal protein L18a | AI719134, AA047273, |
| [symbol] | [symbol] | | 5.2 | 0.000 | 4.0 | 20.9 | 195453 | ribosomal protein S27 (metallopanstimulin 1) | AA525023, AA555149, |
| [symbol] | [symbol] | | 5.1 | 0.000 | 2.5 | 12.5 | 252259 | ribosomal protein S3 | AA708649, AI792490, |
| [symbol] | [symbol] | | 4.8 | 0.000 | 2.0 | 9.7 | 75538 | ribosomal protein S7 | M77233, AA513485, |
| [symbol] | [symbol] | | 4.7 | 0.000 | 2.7 | 12.5 | 177415 | ribosomal protein S30; Finkel-Biskis- [symbol] murine sarcoma virus (FBR-MuSV) [symbol] | AA025263, AA055838, |
| [symbol] | [symbol] | | 4.7 | 0.000 | 1.8 | 8.3 | 75879 | ribosomal protein L19 | AI219423, AA075993, |
| [symbol] | [symbol] | | 4.6 | 0.000 | 2.2 | 10.2 | 180946 | ribosomal protein L5 | W95798, AA491764, |
| [symbol] | [symbol] | * | 4.3 | 0.000 | 21.5 | 91.4 | 151004 | ribosomal protein S8 | AI357743, AW051118, |
| [symbol] | [symbol] | | 4.1 | 0.000 | 5.4 | 22.3 | [symbol] | ribosomal protein L28 | AA225838, AA480489, |
| [symbol] | [symbol] | | 4.0 | 0.000 | 3.1 | 12.5 | 184014 | ribosomal protein L31 | AA340384, AA024502, |
| [symbol] | [symbol] | | 4.0 | 0.000 | 7.6 | 30.1 | 180842 | ribosomal protein L13 | AI808885, AI992115, |
| 39 | CTGTTGGTGA | | 3.9 | 0.000 | 4.0 | 15.8 | 3463 | ribosomal protein S23 | AW020385, AW022474, |
| 40 | AGGGCTTCCA | | 3.8 | 0.000 | 5.1 | 19.5 | 29797 | ribosomal protein L10 | AA244126, AA508388, |
| 41 | TAAGGAGCTG | | 3.8 | 0.000 | 3.8 | 14.4 | 77904 | ribosomal protein S26 | AW022428, X69654, |
| 42 | GTGAAGGCAG | | 3.7 | 0.000 | 3.4 | 12.5 | 77039 | ribosomal protein S3A | AA070817, AA074191, |
| 43 | CGCCGGAACA | | 3.4 | 0.000 | 3.8 | 13.0 | 286 | ribosomal protein L4 | AL119264, AW021149, |
| 44 | AGGCTACGGA | | 3.4 | 0.000 | 8.7 | 29.7 | 119122 | ribosomal protein L13a | AA190354, AW328422, |
| 45 | CCTTCGAGAT | | 3.3 | 0.000 | 4.0 | 13.5 | 76194 | ribosomal protein S5 | AI971757, AA057826, |
| 46 | AAGACAGTGG | | 3.3 | 0.000 | 9.2 | 30.1 | 184109 | ribosomal protein L37a | AA353060, AW020311, |
| 47 | TTACCATATC | | 3.3 | 0.000 | 6.9 | 22.7 | 177461 | ribosomal protein L39 | AW023657, T40220, |
| 48 | TGTGCTAAAT | | 3.2 | 0.000 | 5.1 | 16.7 | 250695 | ribosomal protein L34 | AW020881, N85940, |
| 49 | GCCGTGTCCG | | 3.2 | 0.001 | 2.9 | 9.3 | 241507 | ribosomal protein S6 | AA148286, AA167421, |
| 50 | GGCAAGAAGA | | 3.0 | 0.000 | 3.8 | 11.6 | 111611 | ribosomal protein L27 | AA531226, [symbol] , |
| 51 | ACATCATCGA | | 3.0 | 0.000 | 7.4 | 22.3 | 182979 | ribosomal protein L12 | L06505, AA484263, |
| 52 | GGATTTGGCC | | 2.9 | 0.000 | 16.8 | 48.2 | 251247 | ribosomal protein, large P2 | AA037465, AA064832, |
| 53 | CGCTGGTTCC | | 2.8 | 0.000 | 8.5 | 24.1 | 179943 | ribosomal protein L11 | AA226392, AA230322, |
| 54 | TACAAGAGGA | | 2.8 | 0.006 | 2.7 | 7.4 | 174131 | ribosomal protein L6 | AI042168, D17554 |
| 55 | TTGGTCCTCT | | 2.7 | 0.000 | 18.4 | 50.1 | 108124 | ribosomal protein L41 | Z12962, AA045319, |
| 56 | CTCCTCACCT | | 2.6 | 0.001 | 4.9 | 13.0 | 119122 | ribosomal protein L13a | AW242158, AW245640, |
| 57 | AATAGGTCCA | | 2.5 | 0.000 | 9.2 | 23.2 | 113029 | ribosomal protein S25 | AA228780, AA229897, |
| 58 | GAGGGAGTTT | | 2.5 | 0.000 | 18.6 | 46.8 | 76064 | ribosomal protein L27a | AA228189, AA229949, |
| 59 | AAGAAGATAG | | 2.5 | 0.002 | 4.3 | 10.7 | 184776 | ribosomal protein L23a | AA040728, AA888884, |
| 60 | ATTATTTTTC | | 2.5 | 0.006 | 3.6 | 8.8 | 153 | ribosomal protein L7 | AA138446, AW020191, |
| 61 | ACTCCAAAAA | | 2.2 | 0.000 | 10.3 | 22.7 | 133230 | ribosomal protein S15 | AA079663, AA151459, |
| | SECTION 2. | | | | | | | PROTEIN SYNTHESIS | |
| 62 | CTGGCGAGCG | | 18.6 | 0.000 | 0.2 | 4.2 | 174070 | [symbol] carrier protein | AA211097, AA283711, |
| 63 | GAGCGGGATG | | 16.6 | 0.001 | 0.2 | 3.7 | 77080 | proteasome (prosome, macropain) subunit beta type, 6 | D29012, X61971, |
| 64 | GCATAGGCTG | | 12.4 | 0.000 | 0.4 | 5.6 | 12084 | Tu translation elongation factor, mitochondrial | AL037768, L38995, |
| 65 | GGCTCCCACT | | 11.7 | 0.000 | 0.7 | 7.9 | 74335 | heat shock 90 kD protein 1, beta | AW023752, AA034511, |
| 66 | GCCCAGCTGG | | 11.4 | 0.000 | 0.4 | 5.1 | 223241 | eukaryotic translation elongation factor 1 delta (guanine nucleotide exchange protein) | Z21507, AA489523, |
| 67 | TGAAATAAAC | * | 10.4 | 0.004 | 0.0 | 2.3 | 173205 | nucleophosmin (nucleolar phosphoprotein B23, numatin) | AI184619 |
| 68 | TACCAGTGTA | | 10.4 | 0.004 | 0.0 | 2.3 | 79037 | heat shock 60 kD protein 1 (chaperonin) | AW021205, AW103323, |
| 69 | TGTGTTGAGA | | 9.4 | 0.000 | 5.6 | 52.4 | 181165 | eukaryotic translation elongation factor 1 alpha 1 | AA630271, AA668532, |
| 70 | TGGGCAAAGC | | 9.0 | 0.000 | 2.0 | 18.1 | 2188 | eukaryotic translation elongation factor 1 gamma | AA038923, AA190762, |
| 71 | ACATCCTCAC | | 8.3 | 0.010 | 0.0 | 1.9 | 18700 | proteasome (prosome, macropain) 26S subunit, non-ATPase, 13 | AA024838, AA149127, |
| 72 | TAAAATTTGT | * | 8.3 | 0.010 | 0.0 | 1.9 | 93379 | eukaryofic translation initiation factor 4B | AA054750, AA133563, |
| 73 | CAGTCTAAAA | | 8.3 | 0.003 | 0.4 | 3.7 | 76118 | ubiquitin carboxyl-terminal esterase L1 ( [symbol] thiolesterase) | X04741, AA029783, |
| 74 | GAAGGCATCC | | 8.3 | 0.010 | 0.0 | 1.9 | 250758 | proteasome (prosome, macropain) 28S subunit, ATPase, 3 | F32284, AA130329, |
| 75 | TGGCTAGTGT | | 6.9 | 0.001 | 0.7 | 4.6 | 118865 | proteasome (prosome, macropain) subunit, beta type, 7 | AA804284, AA829376, |
| 76 | CAGATCTTTG | | 5.8 | 0.000 | 1.1 | 6.5 | [symbol] | ubiquitin A-52 residue ribosomal protein fusion product 1 | AF075321, AI110823, |
| 77 | TCACAAGCAA | | 4.4 | 0.001 | 1.6 | 7.0 | 146763 | nascent-polypeptide-associated complex alpha polypeptide | AF054187, X80909, |
| 78 | AACTCTTGAA | | 3.6 | 0.006 | 1.3 | 5.1 | 58189 | eukaryotic translation initiation factor 3, subunit 3 (gamma, 40 kD) | U54559, AA024720, |
| 79 | AGCACCTCCA | | 3.5 | 0.000 | 9.0 | 31.5 | 75309 | eukaryotic translation elongation factor 2 | AA229607, AA533837, |
| 80 | AGCCCTACAA | * | 3.4 | 0.000 | 15.4 | 52.9 | 180532 | heat shock 90 kD protein 1, alpha | AW088888, AW128905, |
| 81 | CGCCGCGGTG | | 2.9 | 0.004 | 2.7 | 7.9 | 4835 | eukaryotic translation initiation factor 3, subunit 8 (110 kD) | AA573953, AA778265, |
| 82 | TTCATACACC | * | 2.6 | 0.000 | 31.3 | 82.8 | 180532 | heat shock 90 kD protein 1, alpha | AW080984 |
| 83 | TGAAATAAAA | | 2.5 | 0.000 | 10.1 | 25.5 | 173205 | nucleophosmin (nucleolar phosphoprotein B23, numatrin) | AI926288, AW166350, |
| 84 | ATCAGTGGCT | | 2.5 | 0.009 | 3.1 | 7.9 | 89545 | proteasome (prosome, macropain) subunit, beta type, 4 | R25997, R68878, |
| 85 | CCCTGATTTT | | −7.1 | 0.000 | 9.8 | 1.4 | 183684 | eukaryotic translation initiation factor 4 gamma, 2 | U73824, U76111, |
| 86 | AAGAGGTTTG | | −9.2 | 0.001 | 4.3 | 0.0 | 74368 | transmembrane protein (63 kD), endoplasmic reticulum/Golgi intermediate compartment | X69910, AI131495, |
| 87 | GTTTTTGCTT | | 2.4 | 0.075 | 1.6 | 3.7 | 79110 | Nucleolin | AA133588, AA135423, A |
| 88 | TACAAAACCA | | 2.1 | 0.923 | 0.9 | 1.9 | 79110 | Nucleolin | AA088423, AA284953, A |
| | SECTION 3. | | | | | | | GENES INVOLVED IN METASTASIS | |
| 89 | AATAGAAATT | | 14.5 | 0.000 | 0.0 | 3.2 | 313 | secreted phosphoprotein 1 (osteopontin, bone sialoprotein 1, early T-lymphocyte activation 1) | AW021049, AA021512, |
| 90 | ATGCTCCCTG | | 14.5 | 0.000 | 0.0 | 3.2 | 79339 | lactin, galactoside-binding, soluble, 3 binding protein (galectin 6 binding protein) | L13210, X79089 |
| 91 | ACAGGGTGAC | | 14.5 | 0.000 | 0.0 | 3.2 | 174050 | endothelial differentiation-related factor 1 | AA975055, AA992919, |
| 92 | AACGCGGCCA | | 12.8 | 0.000 | 1.1 | 14.4 | 73798 | macrophage migration inhibitory factor (glycosylation-inhibiting factor) | AA523321, AA927264 |
| 93 | AAGAAAGGAG | | 11.4 | 0.000 | 0.9 | 10.2 | 202097 | procollagen C-endopeptidase enhancer | L33799 |
| 94 | GTAAGTCTCA | | 8.3 | 0.010 | 0.0 | 1.9 | 211584 | neurofilament, light polypeptide (68 kD) | AW022557, AA330627, |
| 95 | GCCGATCCTC | | 8.3 | 0.010 | 0.0 | 1.9 | 24930 | tubulin-specific chaperone a | AF038952 |
| 96 | GGCTCCTGGC | | 8.3 | 0.010 | 0.0 | 1.9 | 5215 | integrin beta 4 binding protein | AF022229, AF047433, |
| 97 | GCCGGGTGGG | | 7.8 | 0.001 | 0.7 | 5.1 | 74631 | basigin | D45131, L10240, |
| 98 | GGGGAAATCG | | 5.9 | 0.000 | 3.8 | 22.3 | 76293 | thymosin, beta 1D | AA147288, AA155816, |
| 99 | GCCCCCAATA | | −1.7 | 0.007 | 20.1 | 11.6 | 227751 | lectin, galactoside-binding, soluble, 1 (galectin 1) | J04456, X14829, |
| 100 | ACCAAAAACC | | −1.8 | 0.003 | 21.3 | 11.6 | 172928 | collagen, type I, alpha 1 | AA454809, AA454820, |
| 101 | GTTGTGGTTA | | −2.2 | 0.000 | 30.0 | 13.9 | 75415 | beta-2-microglobulin | AB021288, AA897072, |
| 102 | AACTGCTTCA | | −2.5 | 0.002 | 12.8 | 5.1 | 11538 | actin related protein 2/3 complex, subunit fB (41 kD) | AA031434, AA045773, |
| 103 | TCTCTGATGC | | −2.7 | 0.001 | 12.5 | 4.6 | 6441 | tissue inhibitor of [symbol] [symbol] 2 | AL110197, AL134962, |
| 104 | TGTACCTGTA | | −2.8 | 0.000 | 14.1 | 5.1 | 169478 | tubulin, alpha, ubiquitous | AA018607, AA018627, |
| 105 | GCTTTATTTG | | −2.9 | 0.000 | 15.9 | 5.6 | 180952 | actin, beta | AA419273, AA528145, |
| 106 | TCCTGTAAAG | | −3.1 | 0.007 | 7.2 | 2.3 | 74034 | caveolin 1, caveolae protein, 22 kD | AA034380, AA044994, |
| 107 | TGCTAAAAAA | | −4.7 | 0.000 | 17.5 | 3.7 | 146550 | myosin, heavy polypeptide 9, non-muscle | AA041529, AA070528, |
| 108 | TGGAAATGAC | | −4.9 | 0.000 | 42.8 | 8.8 | 172928 | collagen, type I, alpha 1 | AA853342, AA436411, |
| 109 | TGGAAATGCC | | −5.0 | 0.001 | 6.9 | 1.4 | 172928 | collagen, type I, alpha 1 | AI190987, AI339782, |
| 110 | GTTTTTTTTA | * | −5.5 | 0.004 | 5.1 | 0.9 | 179573 | collagen, type I, alpha 2 | AI825593 |
| 111 | ACAGGCTACG | | −6.0 | 0.002 | 5.6 | 0.9 | 75777 | [symbol] | AI188763, AI540294, |
| 112 | TAAAAATGTT | | −6.3 | 0.000 | 17.5 | 2.8 | 82085 | plasminogen activator inhibitor, type I | M14083, AA040151, |
| 113 | GTTTCTAATA | | −6.3 | 0.006 | 2.9 | 0.0 | 239298 | microtubule-associated protein 4 | AA043400, AA086286, |
| 114 | TTAAAGATTT | | −7.1 | 0.000 | 13.2 | 1.9 | 77899 | tropomyosin 1 (alpha) | AA026364, AA036782, |
| 115 | GACCGCAGGA | | −7.2 | 0.003 | 3.4 | 0.0 | 119129 | collagen, type IV, alpha 1 | X03883, AW020005, |
| 116 | TAATCCTCAA | | −8.2 | 0.000 | 7.6 | 0.9 | 78409 | collagen, type XVIII, alpha 1 | AA009987, AA099281, |
| 117 | TGTAGAAAAA | | −9.2 | 0.004 | 4.3 | 0.5 | 119076 | tubulin, beta polypeptide | [symbol] [symbol] , AI623884, |
| 118 | TTAGTGTCGT | | −10.6 | 0.002 | 4.9 | 0.5 | 111779 | secreted protein, acidic, cystaine-rich (osleonectin) | AW020585, AW022769, |
| 119 | GCCCCAATAA | * | −11.1 | 0.000 | 5.1 | 0.0 | 227751 | lectin, galactoside-binding, soluble, 1 (galectin I) | AA095630 |
| 120 | AAAGTCATTG | * | −11.1 | 0.001 | 5.1 | 0.5 | 77899 | tropomyosin 1 (alpha) | M19713 |
| 121 | TGCAATATGC | | −12.5 | 0.000 | 5.8 | 0.0 | 750 | fibrillin 1 (Marian syndrome) | L13923, X63556, |
| 122 | ATCTTGTTAC | | −14.5 | 0.000 | 6.7 | 0.5 | 116162 | fibronectin 1 | AW020421, AW020447, |
| 123 | AAAAAGCTGC | | −15.9 | 0.000 | 7.4 | 0.0 | 111779 | secreted protein, acidic, cysteine-rich (osteonectin) | AA987875, AI755199, |
| 124 | AAAATATTTT | | −18.8 | 0.000 | 8.7 | 0.0 | 119000 | actinin, alpha 1 | X55187, AA024895, |
| 125 | ATGTGAAGAG | | −19.9 | 0.000 | 268.2 | 13.5 | 111779 | secreted protein, acidic, cysteine-rich (osteonectin) | AA228362, AA852763, |
| 126 | TGGCCTAATA | | −27.5 | 0.000 | 12.8 | 0.0 | 1501 | syndecan 2 (heparan sulfate proteoglycan 1, cell surface-associated, fibroglycan) | AA873519, AI279414, |
| 127 | TTTGCACCTT | | −37.6 | 0.000 | 17.5 | 0.5 | 75511 | connective tissue growth factor | AW021964, U14750, |
| | SECTION 4. | | | | | | | GLYCOLYSIS ENZYMES | |
| 128 | TAGCTTCTTC | | 14.5 | 0.000 | 0.0 | 3.2 | 76392 | aldehyde dehydrogenase 1, soluble | AA911018, AI150648, |
| 129 | ACCTTGTGCC | | 10.4 | 0.004 | 0.0 | 2.3 | 878 | sorbitol dehydrogenase | AA570172, AA570189, |
| 130 | TCTGCTTGTC | | 10.4 | 0.004 | 0.0 | 2.3 | 76392 | aldehyde dehydrogenase 1, soluble | H79748, H05271, |
| 131 | TGACTGAAGC | | 10.4 | 0.004 | 0.0 | 2.3 | 3343 | 3-phosphoglycerate dehydrogenase | AA742550, AA113268, |
| 132 | TGGCCCCACC | | 7.5 | 0.000 | 1.1 | 8.3 | 198281 | pyruvate kinase, muscle | AA766601, AA768285, |
| 133 | GCGACCGTCA | | 7.3 | 0.000 | 0.9 | 6.5 | 183760 | aldolase A, fructose-bisphosphate | AA169762, AA557193, |
| 134 | TACCATCAAT | | 4.7 | 0.000 | 5.8 | 27.4 | 195188 | glyceraldehyde-3-phosphate dehydrogenase | AA226658, AA522734, |
| 135 | TGAGGGAATA | | 4.4 | 0.000 | 2.2 | 9.7 | 83848 | triosephosphate isomerase 1 | AA587096, AA587188, |
| | SECTION 5. | | | | | | | MITOCHONDRIAL FUNCTION PROTEINS | |
| 136 | GAATCGGTTA | | 22.8 | 0.000 | 0.2 | 5.1 | 80595 | NADH dehydrogenase (ubiquinone) Fe-S protein 5 (15 kD) (NDUFB4) | AF047434, AA457600, |
| 137 | GGGGGTCACC | | 18.6 | 0.000 | 0.0 | 4.2 | 80986 | ATP synthase, H+ transporting, mitochondrial F0 complex, subunit c (subunit 9), isoform 1 | AA659764, AA866065, |
| 138 | AAGGAGTTTG | | 14.5 | 0.000 | 0.0 | 3.2 | 661 | NADH dehydrogenase (ubiquinone) 1 beta subcomplex, 7 (18 kD, B18) | AA493442, AA832435, |
| 139 | AGGTCCTAGC | | 14.5 | 0.000 | 0.4 | 6.5 | 226795 | glutathione S-transferase pi | F34719, U30897, |
| 140 | CTTAGAGCCC | | 12.4 | 0.006 | 0.2 | 2.8 | 211929 | thioredoxin, mitochondrial | AI680239 |
| 141 | TTCTGGCTGC | | 12.4 | 0.006 | 0.2 | 2.8 | 119251 | ubiquinol-cytochrome c reductase core protein 1 | F28971, AI005342, |
| 142 | GTGGTACAGG | | 7.3 | 0.007 | 0.4 | 3.2 | 31731 | ESTs, Highly similar to PUTATIVE PEROXISOMAL ANTIOXIDANT ENZYME [H. sapiens] | AA745952, AA829707, |
| 143 | GTGACAACAC | | 5.2 | 0.003 | 0.9 | 4.6 | 149155 | voltage-dependent anion channel 1 | AA907238, AI075271, |
| 144 | GCCTGCTGGG | | 4.8 | 0.000 | 2.0 | 9.7 | [symbol] | glutathione peroxidase 4 (phospholipid hydroperoxidase) | X71973, AA743017, |
| 145 | GGGGACTGAA | | 4.6 | 0.003 | 1.1 | 5.1 | 3709 | low molecular mass ubiquinone-binding protein (9.5 kD) | AA010879, AA025093, |
| 146 | CCCATCGTCC | * | 4.3 | 0.000 | 21.5 | 91.4 | mito | cytochrome c oxidase II (Welle et al., 1999), Tag matches mitochondrial sequences | AA069405, AA074137 |
| 147 | TGATTTCACT | * | 4.1 | 0.000 | 4.0 | 18.7 | mito | cytochrome c oxidase III (Welle et al., 1999), Tag matches mitochondrial sequences | AA757623 |
| 148 | TGAAGGAGCC | | 4.1 | 0.007 | 1.1 | 4.6 | 89399 | ATP synthase, H+ transporting, mitochondrial F0 complex, subunit c (subunit 9), isoform 2 | F27013, D13119, |
| 149 | GTGACCTCCT | | 4.1 | 0.003 | 1.3 | 5.6 | 81097 | cytochrome c oxidase subunit VIII | F28051, F28676, |
| 150 | AGCCCTACAA | * | 3.4 | 0.000 | 15.4 | 52.9 | mito | NADH dehydrogenase 3 (Welle et al., 1999), Tag matches mitochondrial sequences | AA577697, AI022799 |
| 151 | TTCATACACC | * | 2.6 | 0.000 | 31.3 | 82.6 | mito | NADH dehydrogenase 4/4L (Welle et al., 1999), Tag matches mitochondrial sequences | AA971178 |
| 152 | CACCTAATTG | * | 2.3 | 0.000 | 34.2 | 79.3 | mito | ATPase 6/8 (Welle et al., 1999), Tag matches mitochondrial sequences | AA031407 |
| 153 | GAGAGCTCCC | | −2.6 | 0.008 | 7.8 | 2.8 | 169919 | electron-transfer-flavoprotein, alpha polypeptide (glutaric aciduria II) | AI364921 |
| 154 | GTAAGATTAG | * | −5.8 | 0.000 | 8.1 | 1.4 | 5417 | oxygen regulated protein (150 kD) | AA828743 |
| 155 | GCCCCAATAA | * | −11.1 | 0.000 | 5.1 | 0.0 | 173554 | ubiquinol-cytochrome c reductase core protein II | AA425586 |
| | SECTION 6. | | | | | | | OTHER GENES | |
| 156 | CCCCCTGGAT | | 21.8 | 0.000 | 0.4 | 9.7 | N/A | calcyclin | AI832624, AA081048, |
| 157 | GTGCGCTAGG | | 18.6 | 0.000 | 0.2 | 4.2 | 9408 | ESTs, classified Into serine/threonine kinase, Highly similar to The KIAA0151 gene product is | AA420781, AA492367, |
| 158 | GAGTGGGGGC | | 18.6 | 0.000 | 0.2 | 4.2 | 14089 | ESTs, Weakly similar to LYSOSOMAL PRO-X CARBOXYPEPTIDASE PRECURSOR [H.s | AA362125, AA418395, |
| 159 | TTTCCTTCCT | * | 16.6 | 0.001 | 0.2 | 3.7 | 104143 | [symbol] [symbol] , light polypeptide (Lca) | AA574409, AA737504, |
| 160 | ATAAGATACA | | 16.6 | 0.000 | 0.0 | 3.7 | 199026 | RAS p21 protein activator (GTPase activating protein) 3 (Ins(1,3,4,5)P4-binding protein) | X69399 |
| 161 | AACGCTGCCT | | 14.5 | 0.000 | 0.0 | 3.2 | 28914 | adenine phosphoribosyltransferase | AA444001, AA535523 |
| 162 | ATTTGTCCCA | | 12.4 | 0.000 | 0.4 | 5.6 | 139800 | high-mobility group (nonhistone chromosomal) protein Isoforms I and Y | AA071304, AA074087, |
| 163 | TCAGACGCAG | | 12.4 | 0.006 | 0.2 | 2.8 | 182371 | prothymosin, alpha (gene sequence 28) | AW129309 |
| 164 | TCTCTTTTTC | * | 12.4 | 0.006 | 0.2 | 2.8 | 119529 | epididymal secretory protein (19.5 kD) | AA025798, AA039545, |
| 165 | TTTCAGGGGA | * | 12.4 | 0.006 | 0.2 | 2.8 | 2853 | poly(rC)-binding protein 1 | AI312897 |
| 166 | CGGCCCAACG | | 12.4 | 0.001 | 0.0 | 2.8 | 20521 | HMTI (hnRNP methyltransferase, S, cerevisiae)-like 2 | AA031375, AA187773, |
| 167 | TTCTCCCGCT | | 12.4 | 0.001 | 0.0 | 2.8 | 118126 | protective protein for beta-galactosidase (galactosalidosis) | M22960, AI752680, |
| 168 | GGGGGACGGC | | 12.4 | 0.001 | 0.0 | 2.8 | 21346 | ESTs, Weakly similar to F42C5.7 gene product (C. elegans) | AA015864, AA026165, |
| 169 | GGCCCTGAGC | | 11.4 | 0.000 | 0.4 | 5.1 | 71618 | polymerase (RNA) II (DNA directed) polypeptide L (7.6 kD), Human RNA polymerase II subu | AA570105, AA562657, |
| 170 | TATGTGATTT | | 10.4 | 0.000 | 0.4 | 4.6 | 5216 | ESTs, Highly similar to HSPC028 [H. sapiens] | AA713577, AA748622, |
| 171 | GGCAGAGGAC | | 10.4 | 0.000 | 0.7 | 7.0 | 118638 | non-metastatic cells 1, protein (NM23A) expressed in | X17620, AA046312, |
| 172 | GCCAAGATGC | | 10.4 | 0.004 | 0.0 | 2.3 | 83135 | p53-responsive gene 6 | AA038471, AA149617, |
| 173 | CCCACACTAC | | 10.4 | 0.004 | 0.0 | 2.3 | 242024 | guanine nucleotide binding protein (G protein), beta polypeptide 2 | M16538, AA548194, |
| 174 | CCGTCATCCT | | 10.4 | 0.004 | 0.0 | 2.3 | 153591 | Not56 (D. melanogaster)-like protein | AI914552, AA412507, |
| 175 | TGAAATAAAC | * | 10.4 | 0.004 | 0.0 | 2.3 | 155212 | methylmalonyl Coenzyme A mutase | AI569812, AI628986, |
| 176 | TGAAATAAAC | * | 10.4 | 0.004 | 0.0 | 2.3 | 238380 | Human endogenous retroviral protease mRNA, complete cds | AA527289 |
| 177 | GACCCTGCCC | | 10.4 | 0.004 | 0.0 | 2.3 | 173464 | FK506-binding protein 8 (38 kD) | L37033, AA587607, |
| 178 | CAGCAGAAGC | | 9.8 | 0.000 | 0.9 | 8.8 | 256313 | pinin, desmosome associated protein | N76807 |
| 179 | CTCATAGCAG | * | 9.3 | 0.001 | 0.4 | 4.2 | 103636 | chromosome 1 open reading frame 9 | AI312752, AI312755 |
| 180 | CTCATAGCAG | * | 9.3 | 0.001 | 0.4 | 4.2 | 119252 | tumor protein, translationally-controlled 1 | N92214, AA045631, |
| 181 | CTGGGCCTGG | | 8.3 | 0.010 | 0.0 | 1.9 | 74573 | similar to vaccinia virus [symbol] [symbol] KAL ORF | N99342, D45708, |
| 182 | GGGGTAAGAA | | 8.3 | 0.010 | 0.0 | 1.9 | 80423 | prostatic binding protein | AA613924, AA825254, |
| 183 | GGGCTGGGCC | | 8.3 | 0.010 | 0.0 | 1.9 | 100071 | 6-phosphogluconolactonase | AI217069, AI279087, |
| 184 | TTTTTTGTAA | | 8.3 | 0.010 | 0.0 | 1.9 | 97858 | SH3-domain binding protein 1 | AA748769 |
| 185 | GCAGTGGCCT | | 8.3 | 0.010 | 0.0 | 1.9 | 184276 | solute carrier family 9 (sodium/hydrogen exchanger), Isoform 3 regulatory factor 1 | AA425299, AA593621, |
| 186 | CCCCCAATGC | | 8.3 | 0.010 | 0.0 | 1.9 | 115232 | Spliceosome protein SAP-62 | AI280056, AI348200, |
| 187 | CTGCTGTGAT | * | 8.3 | 0.010 | 0.0 | 1.9 | 1063 | small nuclear [symbol] [symbol] polypeptide C | AA089406, AA099919, |
| 188 | CAGTTGGTTG | | 8.3 | 0.010 | 0.0 | 1.9 | 155218 | E1B-55 kDa-associated protein 5 | AA130531, AA155800, |
| 189 | ATCCATAGTG | | 8.3 | 0.010 | 0.0 | 1.9 | 66772 | TATA box binding protein (TBP)-associated factor, RNA polymerase II, N, 68 kD (RNA- [symbol] | AA662359, AA857343, |
| 190 | CTGGATGCCG | | 8.3 | 0.010 | 0.0 | 1.9 | 106061 | RD RNA-binding protein | AA569818, AA988602, |
| 191 | CTGACCCCCT | | 8.3 | 0.010 | 0.0 | 1.9 | 26492 | beta-1,3- [symbol] [symbol] 3 (glucuronosyltransferase I) | AB009598, AJ005865, |
| 192 | CCTGTACCCC | * | 8.3 | 0.010 | 0.0 | 1.9 | 32317 | Sox-like transcriptional factor | AA045957, AA196459, |
| 193 | TAAAATTTGT | * | 8.3 | 0.010 | 0.0 | 1.9 | 32317 | Sox-like transcriptional factor | AA919117 |
| 194 | TGGCCTGCCC | | 8.3 | 0.010 | 0.0 | 1.9 | 181002 | MLL septin-like fusion | AA761307, AA831791, |
| 195 | TGGCCTCCCC | | 8.3 | 0.010 | 0.0 | 1.9 | 159161 | Rho GDP dissociation Inhibitor (GDI) alpha | X69550 |
| 196 | TGCAGCGCCT | | 8.3 | 0.010 | 0.0 | 1.9 | 77573 | [symbol] [symbol] phosphorylase | X90858, AI018589, |
| 197 | TGAGGGGTGA | | 8.3 | 0.010 | 0.0 | 1.9 | 252979 | G protein pathway [symbol] [symbol] 1 | AA521025, AA569807, |
| 198 | TGAGGCCAGG | * | 8.3 | 0.010 | 0.0 | 1.9 | 79162 | structure specific recognition protein 1 | AW328290, M86737, |
| 199 | GAGAGAAGAG | | 8.3 | 0.010 | 0.0 | 1.9 | 13476 | UDP-GalbetaGlcNAc beta 1,4-galactosyltransferase, polypeptide 3 | AA721091, AA743639, |
| 200 | TCTTCTCACA | * | 8.3 | 0.010 | 0.0 | 1.9 | 656 | cell division cycle 25C | AA206499, AA534482, |
| 201 | GCCGCTACTT | | 8.3 | 0.010 | 0.0 | 1.9 | 32989 | [symbol] [symbol] receptor-like receptor activity modifying protein 1 | AI951585, AJ001014 |
| 202 | CCCTCCTCCG | | 8.3 | 0.010 | 0.0 | 1.9 | 81131 | guanidinoacetate N-methyltransferase | D59710, AI123221, |
| 203 | TATGACCACA | * | 8.3 | 0.010 | 0.0 | 1.9 | 6650 | vacuolar protein sorting 45B (yeast homolog) | AA765898, AA769317, |
| 204 | TACATTCACC | | 8.3 | 0.010 | 0.0 | 1.9 | 82043 | D123 gene product | D14876, U27112, |
| 205 | AAGCGGGACC | | 8.3 | 0.010 | 0.0 | 1.9 | 153438 | N-acetyltransferase, homolog of S. cerevisiae ARD1 | X77588, AA158247, |
| 206 | GATCAATGGA | * | 8.3 | 0.010 | 0.0 | 1.9 | 3090 | EphB1 | AA449788, AA640161, |
| 207 | GATCAATGGA | * | 8.3 | 0.010 | 0.0 | 1.9 | 251788 | glucosamine-6-phosphate deaminase | AA031910, AA151768, |
| 208 | GCCGCCATCT | | 8.3 | 0.010 | 0.0 | 1.9 | 89643 | [symbol] [symbol] ( [symbol] [symbol] -Korsakoff syndrome) | U55017 |
| 209 | CCCTGGGTTC | | 7.7 | 0.000 | 2.5 | 19.0 | 111334 | ferritin, light polypeptide | M11147, M12938, |
| 210 | CAGCCTTGGA | | 7.3 | 0.007 | 0.4 | 3.2 | 65648 | RNA binding motif protein 8 | AA045586, AA188655, |
| 211 | ACCCTTCCCT | * | 7.3 | 0.007 | 0.4 | 3.2 | 99528 | ESTs, Weakly similar to VON EBNER'S GLAND PROTEIN PRECURSOR [H. sapiens] | AA936288, AA977608, |
| 212 | GAGGGGAAAC | | 7.3 | 0.007 | 0.4 | 3.2 | 81972 | SHC (Src homology 2 domain-containing) transforming protein 1 | AA767918, X68148, |
| 213 | ACCCTTCCCT | * | 7.3 | 0.007 | 0.4 | 3.2 | 74564 | signal sequence receptor, beta (translocon-associated protein beta) | AW024384, D37991, |
| 214 | ACTGGGTCTA | | 6.5 | 0.000 | 1.6 | 10.2 | 250871 | non-metastatic cells 2, protein (NM23B) expressed in | AA514798, AA828464, |
| 215 | TGGAGTGGAG | | 5.8 | 0.000 | 1.1 | 6.5 | 3764 | guanylate kinase 1 | F25667, AA024959, |
| 216 | CCCCTCCCTC | * | 5.5 | 0.008 | 0.7 | 3.7 | 79410 | solute carrier family 4, anion exchanger, member 2 (erythrocyte membrane protein band 3- [symbol] | AA741103, AA767203, |
| 217 | CCCCTCCCTC | * | 6.5 | 0.008 | 0.7 | 3.7 | 74564 | signal sequence receptor, beta (translocon-associated protein beta) | AW083845 |
| 218 | ACAGTGGGGA | | 4.6 | 0.003 | 1.1 | 5.1 | 75839 | zinc finger protein 6 (CMPX1) | AA740738, AA100363, |
| 219 | TGATTTCACT | * | 4.1 | 0.000 | 4.0 | 16.7 | 24322 | ATPase, H+ transporting, lysosomal (vacuolar proton pump) 9 kD | AI065143 |
| 220 | TTATGGGATC | | 4.0 | 0.000 | 3.4 | 13.5 | 5862 | guanine nucleotide binding protein (G protein), beta polypeptide 2-like 1 | M24194, AA480431, |
| 221 | ATAGACATAA | | 2.8 | 0.007 | 2.5 | 7.0 | [symbol] | complement component 1, q subcomponent binding protein | AI916184, AA195312, |
| 222 | CACCTAATTG | * | 2.3 | 0.000 | 34.2 | 79.3 | 181368 | U5 anRNP-specific protein (220 kD), ortholog of S. cerevisiae Prp8p | AW129234, AW151854, |
| 223 | GAAATACAGT | * | −1.8 | 0.006 | 18.6 | 10.2 | 79572 | cathepain D (lysosomal aspartyl protease) | AA046688, AA063376, |
| 224 | GCCTTCCAAT | | −2.0 | 0.004 | 15.9 | 7.9 | 76053 | DEAD/H (Asp-Glu-Ala-Asp/His) box polypeptide 5 (RNA helicase, 68 kD) | AA030969, AW019938, |
| 225 | GTGTGTTTGT | | −2.0 | 0.005 | 15.0 | 7.4 | 118787 | transforming growth factor, beta-induced, 68 kD | M77349, AW021500, |
| 226 | AGCAGATCAG | | −2.2 | 0.004 | 13.2 | 6.0 | 119301 | S100 calcium-binding protein A10 (annexin II ligand, calpactin I, light polypeptide (p11)) | AW022967, AA009605, |
| 227 | CTGCCAAGTT | | −2.6 | 0.003 | 10.3 | 3.7 | 75873 | zyxin | AA040172, AA054721, |
| 228 | TTCTGTGAAT | * | −3.7 | 0.002 | 8.5 | 2.3 | 182183 | caldesmon 1 | AA552208, AA652809, |
| 229 | CTTAATCCTG | | −4.1 | 0.000 | 15.2 | 3.7 | 234433 | ESTs, Weakly similar to transporter protein [H. sapiens] | AA864787, AW021494, |
| 230 | TCTCAATTCT | * | −4.5 | 0.003 | 6.3 | 1.4 | 173497 | Sec23 (S. [symbol] [symbol] ) homolog B | AI581164, AW135796, |
| 231 | GCCCTTTCTC | | −4.5 | 0.000 | 12.5 | 2.8 | 7835 | endocytic receptor (macrophage mannose receptor family) | AA126747, AA405572, |
| 232 | TCTCAATTCT | * | −4.5 | 0.003 | 6.3 | 1.4 | N/A | BB1 | AA410935, AA022580, |
| 233 | TTCTTGTTTT | | −5.3 | 0.006 | 4.9 | 0.9 | 74621 | prion protein (p27-30) Creutzfeld-Jakob disease, Gertsmann-Strausler-Scheinker syndrome | D00015, M13687, |
| 234 | TATGACTTAA | * | −5.3 | 0.006 | 4.9 | 0.9 | 89230 | potassium intermediate/small conductance calcium-activated channel, subfamily N, member | AA285078, AA491238, |
| 235 | GTTTTTTTTA | * | −5.5 | 0.004 | 5.1 | 0.9 | 10114 | ESTs, Weakly similar to protein B [H. sapiens] | AA284721, AA496717, |
| 236 | GTCACAGTCC | | −5.8 | 0.009 | 2.7 | 0.0 | 155321 | serum response factor (c-los serum response element-binding transcription factor) | AA024483, AA041538, |
| 237 | TAAGAAAATG | | −8.3 | 0.008 | 2.9 | 0.0 | 75929 | cadherin 11 (OB-cadherin, osteoblast) | AA258422, AA461076, |
| 238 | TTTTTTAAAA | | −6.3 | 0.006 | 2.9 | 0.0 | 227400 | mitogen-activated protein kinase kinase kinase kinase 3 | AA043537 |
| 239 | TTACTTATAC | * | −6.8 | 0.004 | 3.1 | 0.0 | 159 | tumor necrosis factor receptor superfamily, member 1A | AW138039 |
| 240 | TTACTTATAC | * | −6.8 | 0.004 | 3.1 | 0.0 | [symbol] | wingless-type MMTV integration site family, member 2B | AW129457 |
| 241 | GCTGTTTTGT | | −6.8 | 0.004 | 3.1 | 0.0 | 92186 | KIAA0989 protein | AB023206, AA405541, |
| 242 | CTTTCTTTGA | | −8.2 | 0.001 | 3.8 | 0.0 | 4909 | regulated in glioma | AF052161, AA209488, |
| 243 | AAAAGATACT | | −8.2 | 0.009 | 3.8 | 0.5 | 82071 | Cbp/p300-interacting transactivator, with Glu/Asp-rich carboxy-terminal domain, 2 | AA115949, AA146987, |
| 244 | TCCGTGGTTG | | −8.2 | 0.001 | 3.8 | 0.0 | 79516 | brain acid-soluble protein 1 | AF039656, AA602987, |
| 245 | CATTATAACT | | −8.2 | 0.001 | 3.8 | 0.0 | 84359 | hypothetical protein | AA806434, AA832337, |
| 246 | TGTCATCACA | | −9.2 | 0.004 | 4.3 | 0.5 | 83354 | lysyl oxidase-like 2 | AA126278, AA149435, |
| 247 | TTTTGTTTTG | * | −9.7 | 0.003 | 4.5 | 0.5 | 95583 | [symbol] [symbol] 4 superfamily member (tetraspan NET-7) | AA037844, AA040421, |
| 248 | TACAGAGGGA | | −10.1 | 0.002 | 4.7 | 0.5 | 3776 | zinc finger protein 216 | AA730188, AA814563, |
| 249 | AAGTGAAACA | | −11.1 | 0.001 | 5.1 | 0.5 | 93659 | protein disulfide isomerase related protein (calcium-binding protein, intestinal-related) | AW411440 |
| 250 | AGTTTCCCAA | | −13.0 | 0.000 | 6.0 | 0.5 | 75854 | SULT1C sulfotransferase | AF055584, AA113827, |
| 251 | TACAATAAAC | | −14.0 | 0.000 | 6.5 | 0.0 | 9071 | progesterone membrane binding protein | AA836144, AJ002030, |
| 252 | CATATCATTA | | −15.9 | 0.000 | 7.4 | 0.0 | 119205 | insulin-like growth factor binding protein 7 | [symbol] [symbol] , [symbol] [symbol] , |
| | SECTION 7. | | | | | | | EST clones of unknown function | |
| 253 | GCACCTCAGC | | 14.5 | 0.000 | 0.0 | 3.2 | 10702 | ESTs | AA027098, AA035781, |
| 254 | GAGAGAAAAT | | 14.5 | 0.000 | 0.0 | 3.2 | 181444 | ESTs, Weakly similar to R12C12.6 [C. elegans] | AA476914, AA630706, |
| 255 | ACTTTTTAAA | | 14.1 | 0.000 | 1.1 | 15.8 | 157300 | EST | AI365306 |
| 256 | TTTCAGGGGA | * | 12.4 | 0.006 | 0.2 | 2.8 | 3804 | DKFZP564C1940 protein | AA831451, AA070917, |
| 257 | TGATGGGCAT | | 12.4 | 0.006 | 0.2 | 2.8 | 74284 | ESTs, Moderately similar to S. cerevisiae hypothetical protein L3111 [H. sapiens] | AI688503, AI745361, |
| 258 | CTGGGCGTGT | * | 12.4 | 0.001 | 0.0 | 2.8 | 108946 | ESTs, Weakly similar to laminin beta-2 chain precursor [H. sapiens] | AA046403, AA759123, |
| 259 | ACGGTGATGT | | 12.4 | 0.001 | 0.0 | 2.8 | 10453 | ESTs | AA005401, AA045808, |
| 260 | CTGGGCGTGT | * | 12.4 | 0.001 | 0.0 | 2.8 | 15246 | EST | T90794 |
| 261 | TGAGGCCAGG | * | 8.3 | 0.010 | 0.0 | 1.9 | 110128 | ESTs | AA584364 |
| 262 | GGTTTGTGTG | | 8.3 | 0.010 | 0.0 | 1.9 | 83954 | Homo sapiens unknown mRNA | AF061739, AA732389, |
| 263 | TTGTGGGATC | | 8.3 | 0.010 | 0.0 | 1.9 | 167795 | ESTs | AA018907, AA126960, |
| 264 | CCTGTACCCC | * | 6.3 | 0.010 | 0.0 | 1.9 | 12342 | Homo sapiens clone 24538 mRNA sequence | AI924428 |
| 265 | TATGACCACA | * | 6.3 | 0.010 | 0.0 | 1.9 | 176577 | ESTs | AI651376 |
| 266 | ACTACCTTCA | | 8.3 | 0.010 | 0.0 | 1.9 | 9601 | ESTs, Highly similar to CGI-106 protein [H. sapiens] | AA744772, AA805690, |
| 267 | CTGCTGTGAT | * | 8.3 | 0.010 | 0.0 | 1.9 | 193909 | ESTs, Weakly similar to IIII ALU SUBFAMILY SC WARNING ENTRY IIII (H. sapiens) | AA628209, AJ690704, |
| 268 | GAGTGAGTGA | * | 7.3 | 0.007 | 0.4 | 3.2 | 52186 | ESTs, Weakly similar to IIII ALU SUBFAMILY J WARNING ENTRY IIII (H. sapiens) | H62203 |
| 269 | GAGTGAGTGA | * | 7.3 | 0.007 | 0.4 | 3.2 | 10463 | ESTs, Weakly similar to C44C1.2 gene product (C. elegans) | AA009696, AA088448, |
| 270 | TTTGTTAAAA | * | 4.1 | 0.007 | 1.1 | 4.6 | 111244 | ESTs | AA748351, AA769191, |
| 271 | AAGATAATGC | * | 4.1 | 0.007 | 1.1 | 4.6 | 102898 | ESTs, Weakly similar to C11D2.4 (C. elegans) | AA806449, AA115687, |
| 272 | AAGATAATGC | * | 4.1 | 0.007 | 1.1 | 4.6 | 251978 | EST | C14037 |
| 273 | TTTGTTAAAA | * | 4.1 | 0.007 | 1.1 | 4.6 | 207118 | EST | AJ806514 |
| 274 | AGGAAAGCTG | | 4.0 | 0.000 | 6.3 | 25.0 | 76437 | DKFZP5668023 protein | AA523344, AA551986, |
| 275 | CAAGCATCCC | | 3.5 | 0.000 | 5.8 | 20.4 | 153423 | ESTs | AW275649, AW276934, |
| 276 | GAAATACAGT | * | −1.8 | 0.006 | 18.6 | 10.2 | 67201 | ESTs | AA122047, AA404659, |
| 277 | TTCTGTGAAT | * | −3.7 | 0.002 | 8.5 | 2.3 | 77870 | ESTs | AA022926, AA121431, |
| 278 | GAAATAATGG | * | −4.0 | 0.001 | 9.2 | 2.3 | 178053 | ESTs | AA412270, AJ208372, |
| 279 | GAAATAATGG | * | −4.0 | 0.001 | 9.2 | 2.3 | 209037 | ESTs | AJ143898, AJ808260 |
| 280 | TATGACTTAA | * | −5.3 | 0.006 | 4.9 | 0.9 | 203352 | ESTs | W52993 |
| 281 | GTAAGATTAG | * | −5.8 | 0.000 | 8.1 | 1.4 | 250705 | ESTs | AA953513, AJI28280, |
| 282 | GCCATATTAT | | −5.8 | 0.009 | 2.7 | 0.0 | 19280 | KIAA0544 protein | R70600, AJ081100, |
| 283 | AATTTTCATT | * | −6.3 | 0.008 | 2.9 | 0.0 | 77695 | KIAA0008 gene product | AJ889277 |
| 284 | AATTTTCATT | * | −6.3 | 0.006 | 2.9 | 0.0 | 35092 | ESTs | AA343561, AA449815, |
| 285 | TTCCTCCTTT | * | −7.7 | 0.002 | 3.6 | 0.0 | 226144 | ESTs | AJ215617, AJ982565, |
| 286 | GCTTGTCTTT | | −7.7 | 0.002 | 3.6 | 0.0 | 224620 | ESTs | AJ580377, AJ953171 |
| 287 | ACAGTGTTAA | | −7.7 | 0.002 | 3.6 | 0.0 | 186342 | ESTs | AA167571, AA653213, |
| 288 | TTCCTCCTTT | * | −7.7 | 0.002 | 3.6 | 0.0 | 115581 | EST, Highly similar to KIAA0826 protein (H. sapiens) | AA463908 |
| 289 | TTTTGTTTTG | * | −9.7 | 0.003 | 4.5 | 0.5 | 74867 | ESTs | AA120854, AA489661, |
| 290 | ACAGATTTGA | | −10.1 | 0.000 | 4.7 | 0.0 | 41271 | ESTs | AA253217, AA599448, |
| 291 | TTCCCCCTTC | | −10.6 | 0.000 | 4.9 | 0.0 | 163928 | ESTs | AA577100, AJ819034 |
| 292 | AAAGTCATTG | * | −11.1 | 0.001 | 5.1 | 0.5 | 21145 | Human BAC clone RG083M05 from 7q21-7q22 | AJ056386, AJ161119 |
| | SECTION 8. | | | | | | | UNIDENTIFIED TRANSCRIPTS | |
| 293 | CCGCCGAAGT | | 24.9 | 0.000 | 0.0 | 5.6 | | | |
| 294 | ACCTTTTCAA | | 16.8 | 0.000 | 0.0 | 3.7 | | | |
| 295 | GCCCCTCCGG | | 16.6 | 0.001 | 0.2 | 3.7 | | | |
| 296 | GCTTTCTCAC | | 10.4 | 0.000 | 0.9 | 9.3 | | | |
| 297 | CGCCGCGGCT | | 10.4 | 0.004 | 0.0 | 2.3 | | | |
| 298 | GTGACCACGG | | 9.3 | 0.001 | 0.4 | 4.2 | | | |
| 299 | GTTAACAGTC | | 8.3 | 0.010 | 0.0 | 1.9 | | | |
| 300 | GCCGTTCTTA | | 8.3 | 0.010 | 0.0 | 1.9 | | | |
| 301 | AGGCTACCGG | | 7.3 | 0.007 | 0.4 | 3.2 | | | |
| 302 | ACTTTTTCAC | | 5.4 | 0.000 | 1.8 | 9.7 | | | |
| 303 | TCCCCGTCAT | | −4.5 | 0.003 | 6.3 | 1.4 | | | |
| 304 | AGGAATGTTA | | −5.1 | 0.008 | 4.7 | 0.9 | | | |
| 305 | TCCCTTATTA | | −5.3 | 0.008 | 4.9 | 0.9 | | | |
| 306 | TCTTGATATT | | −5.8 | 0.009 | 2.7 | 0.0 | | | |
| 307 | AAGGCAATTT | | −6.8 | 0.001 | 8.3 | 0.8 | | | |
| 308 | TTCGGTTGGT | | −7.2 | 0.000 | 10.1 | 1.4 | | | |
| 309 | TCCCCGGTAC | | −7.5 | 0.000 | 13.9 | 1.9 | | | |
| 310 | GCTGACGTCA | | −8.2 | 0.001 | 3.8 | 0.0 | | | |
| 311 | TCCCCCGTAC | | −8.7 | 0.001 | 4.0 | 0.0 | | | |
| 312 | ACGTTCTCTT | | −9.2 | 0.001 | 4.3 | 0.0 | | | |
| 313 | TAACTTTTGG | | −9.2 | 0.001 | 4.3 | 0.0 | | | |
| 314 | AAATGCTTGG | | −9.7 | 0.003 | 4.5 | 0.5 | | | |
| 315 | CTAAAAACCT | | −10.1 | 0.000 | 4.7 | 0.0 | | | |
| 316 | GTGAGAGTTT | | −12.1 | 0.000 | 5.6 | 0.5 | | | |
| 317 | GGTGGACACG | | −13.0 | 0.000 | 6.0 | 0.0 | | | |
| 318 | TCCCCTATTA | | −15.4 | 0.000 | 7.2 | 0.0 | | | |
| 319 | CTTTATTCCA | | −18.3 | 0.000 | 17.0 | 0.9 | | | |

TABLE IA
Downstream targets induced by N-myc ribosomal proteins

| Tag sequence | SHEP-2 | SHEP-21N | Fold induction | P value | Unigene Hs. | Gene |
|---|---|---|---|---|---|---|
| GCCGAGGAAG | 1 | 37 | 37.0 | <0.001 | 82148 | ribosomal protein S12 |
| GCTTTTAAGG | 1 | 29 | 29.0 | <0.001 | 8102 | ribosomal protein S20 |
| CCCATCCGAA | 1 | 23 | 23.0 | <0.001 | 91379 | ribosomal protein L26 |
| GGCCGCGTTC | 0 | 23 | >23 | <0.001 | 5174 | ribosomal protein S17 |
| CCAGTGGCCC | 0 | 21 | >21 | <0.001 | 180920 | ribosomal protein S9 |
| GTGTTGCACA | 1 | 16 | 16.0 | <0.001 | 165590 | ribosomal protein S13 |
| GATGCTGCCA | 1 | 16 | 16.0 | <0.001 | 99914 | ribosomal protein L22 |
| CCGTCCAAGG | 2 | 31 | 15.5 | <0.001 | 80617 | ribosomal protein S16 |
| GGAGTGGACA | 1 | 14 | 14.0 | <0.001 | 75458 | ribosomal protein L18 |
| GCCTGTATGA | 2 | 27 | 13.5 | <0.001 | 180450 | ribosomal protein S24 |
| GTTCCCTGGC | 2 | 26 | 13.0 | <0.001 | 177415 | ribosomal protein Fau-S30 |
| ATGGCTGGTA | 6 | 72 | 12.0 | <0.001 | 182426 | ribosomal protein S2 |
| GTGTTAACCA | 1 | 11 | 11.0 | <0.001 | 74267 | ribosomal protein L10 |
| CACAAACGGT | 4 | 43 | 10.8 | <0.001 | 195453 | ribosomal protein S27 |
| CTCAACATCT | 3 | 32 | 10.7 | <0.001 | 73742 | ribosomal protein, large, P0 |
| GTTCGTGCCA | 2 | 18 | 9.0 | <0.001 | 179666 | ribosomal protein L35a |
| GACGACACGA | 4 | 30 | 7.5 | <0.001 | 153177 | ribosomal protein S28 |
| TCGTCTTTAT | 3 | 21 | 7.0 | <0.001 | 75538 | ribosomal protein S7 |
| GGACCACTGA | 5 | 34 | 6.8 | <0.001 | 119598 | ribosomal protein L3 |
| CCTCGGAAAA | 4 | 24 | 6.0 | <0.001 | 2017 | ribosomal protein L38 |
| AATCCTGTGG | 8 | 48 | 6.0 | <0.001 | 178551 | ribosomal protein L8 |
| ATCAAGGGTG | 4 | 21 | 5.3 | <0.001 | 157850 | ribosomal protein L9 |
| GGGCTGGGGT | 20 | 101 | 5.1 | <0.001 | 183698 | ribosomal protein L29 |
| AAGGAGATGG | 5 | 25 | 5.0 | <0.001 | 184014 | ribosomal protein L31/tag matches |
| | | | | | | mitochondrial sequences |
| AAGGTGGAGG | 11 | 55 | 5.0 | <0.001 | 163593 | ribosomal protein L18a |
| TTACCATATC | 10 | 49 | 4.9 | <0.001 | 177461 | ribosomal protein L39 |
| GTGAAGGCAG | 6 | 27 | 4.5 | <0.001 | 77039 | ribosomal protein S3A |
| GAACACATCC | 4 | 18 | 4.5 | 0.002 | 75879 | ribosomal protein L19 |
| CGCCGCCGGC | 10 | 40 | 4.0 | <0.001 | 182825 | human ribosomal protein L35 mRNA |
| GCCGTGTCCG | 5 | 20 | 4.0 | 0.002 | 119213 | ribosomal protein S6 |
| AGGAAAGCTG | 13 | 52 | 4.0 | <0.001 | 76437 | ESTs, highly similar to 60S rpL36 |
| | | | | | | (Rattus norvegicus) |
| CCCCAGCCAG | 7 | 27 | 3.9 | <0.001 | 75459 | ribosomal protein S3 |
| GCAGCCATCC | 13 | 48 | 3.7 | <0.001 | 4437 | ribosomal protein L28 |
| GGCAAGAAGA | 7 | 25 | 3.6 | <0.001 | 111611 | ribosomal protein L27 |
| CCCGTCCGGA | 19 | 65 | 3.4 | <0.001 | 180842 | ribosomal protein L13 |
| CGCTGGTTCC | 15 | 51 | 3.4 | <0.001 | 179943 | ribosomal protein L11 |
| TAAGGAGCTG | 9 | 30 | 3.3 | <0.001 | 77904 | ribosomal protein S26 |
| CCTTCGAGAT | 8 | 26 | 3.3 | 0.001 | 76194 | ribosomal protein S5 |
| GGATTTGGCC | 33 | 103 | 3.1 | <0.001 | 119500 | ribosomal protein, large, P2 |
| AGGCTACGGA | 20 | 63 | 3.2 | <0.001 | 119122 | 60S ribosomal protein L13A |
| CTGCTATACG | 7 | 22 | 3.1 | <0.004 | 180946 | ribosomal protein L5 |
| TGTGCTAAAT | 12 | 35 | 2.9 | <0.001 | 179779 | ribosomal protein L37 |
| GAGGGAGTTT | 34 | 97 | 2.9 | <0.001 | 76064 | ribosomal protein L27a |
| AAGAAGATAG | 8 | 22 | 2.8 | 0.007 | 184776 | ribosomal protein L23a |
| ACATCATCGA | 17 | 46 | 2.7 | <0.001 | 182979 | ribosomal protein L12 |
| CTGTTGGTGA | 12 | 31 | 2.6 | 0.003 | 3463 | ribosomal protein S23 |
| AAGACAGTGG | 26 | 63 | 2.4 | <0.001 | 184109 | ribosomal protein L37a |
| TTGGTCCTCT | 46 | 108 | 2.3 | <0.001 | 108124 | ribosomal protein L41 |
| CTCCTCACCT | 12 | 28 | 2.3 | 0.008 | 119122 | 60S ribosomal protein L13A |
| AATAGGTCCA | 22 | 50 | 2.3 | <0.001 | 113029 | ribosomal protein S25 |
| ACTCCAAAAA | 23 | 46 | 2.0 | 0.004 | 133230 | ribosomal protein S15 |
| CTGGGTTAAT | 47 | 87 | 1.9 | <0.001 | 126701 | ribosomal protein S19 |
| TCAGATCTTT | 38 | 74 | 1.9 | <0.001 | 75344 | ribosomal protein S4, X-linked |
| AGCTCTCCCT | 38 | 56 | 1.9 | 0.003 | 82202 | ribosomal protein L17 |
| TAATAAAGGT | 45 | 78 | 1.7 | 0.002 | 118690 | ribosomal protein S8 |
| TTCAATAAAA | 56 | 89 | 1.6 | 0.004 | 177592 | ribosomal protein, large, P1 |

Additional tags with P values > 0.01 and ≦0.05

| | | | | | | |
|---|---|---|---|---|---|---|
| AAGGTCGAGC | 1 | 8 | 8.0 | 0.022 | 184582 | ribosomal protein L24 |
| CTCGAGGAGG | 0 | 6 | >6 | 0.016 | 3254 | ribosomal protein L23-like |
| GCTCCGAGCG | 0 | 5 | >5 | 0.028 | 80617 | ribosomal protein S16 |
| TACAAGAGGA | 5 | 16 | 3.2 | 0.014 | 174131 | ribosomal protein L6 |
| CCATTGCACT | 7 | 17 | 2.4 | 0.032 | 53798 | ESTs, highly similar to 60S RP L18A |
| CGCCGGAACA | 12 | 27 | 2.3 | 0.012 | 286 | ribosomal protein L4 |
| ATTATTTTTC | 8 | 18 | 2.3 | 0.037 | 153 | ribosomal protein L7 |
| CAATAAATGT | 34 | 53 | 1.6 | 0.027 | 179779 | ribosomal protein L37 |
| CCAGAACAGA | 40 | 60 | 1.5 | 0.030 | 111222 | ribosomal protein L30 |
| GCATAATAGG | 38 | 55 | 1.4 | 0.048 | 184108 | ribosomal protein L21 |

| | | | | | Unigene | | Accession |
|---|---|---|---|---|---|---|---|
| No. | Tag_Sequence | SHEP-2 | SHEP-21N | P value | cluster | Unigene description | code |
| 1 | CGACGAGGAG | 1.8 | 7.0 | 0.0087 | 9999 | epithelial membrane protein 3 | |
| 2 | TTTAAAAAAA* | 21.7 | 7.0 | 0.0003 | 97437 | centriole associated protein | |
| 3 | GAATAAATGT | 7.2 | 1.2 | 0.0053 | 8762 | FK506-binding protein 9 (63 kD) | AI088246 |
| 4 | TAAAATAAAA* | 5.0 | 0.0 | 0.0027 | 87100 | ESTs | |
| 5 | TCTTCTGCCA | 9.5 | 1.7 | 0.0021 | 86392 | ESTs | |
| 6 | TGCTTTGGGA | 1.4 | 5.8 | 0.0100 | 84344 | ESTs, Highly similar to CGI-135 protein [H. sapiens] | AA788733 |
| 7 | GGGCGCTGTG | 0.9 | 6.4 | 0.0020 | 8372 | ubiquinol-cytochrome c reductase (6.4 kD) subunit | |
| 8 | TTAGATAAGC* | 4.5 | 12.2 | 0.0036 | 82916 | chaperonin containing TCP1, subunit 6A (zeta 1) | |
| 9 | AGCTCTCCCT | 29.3 | 53.4 | 0.0000 | 82202 | ribosomal protein L17 | AA961432 |
| 10 | CCCTGCCTTG | 0.0 | 3.5 | 0.0052 | 82045 | midkine (neurite growth-promoting factor 2) | |
| 11 | GCACAAGAAG | 9.6 | 19.2 | 0.0037 | 81634 | ATP synthase, H+ transporting, mitochondrial | |
| | | | | | | F0 complex, subunit b, isoform 1 | |
| 12 | TTAAAGGCCG | 0.5 | 5.2 | 0.0025 | 79086 | ribosomal protein, mitochondrial, L3 | |
| 13 | AGAAAGATGT | 9.5 | 20.3 | 0.0016 | 78225 | annexin A1 | AA846272 |
| 14 | TTTAAAAAAA* | 21.7 | 7.0 | 0.0003 | 77501 | sarcoglycan, beta (43 kD dystrophin-associated glycoprotein) | |
| 15 | GTAAAAAAAA* | 44.7 | 20.9 | 0.0002 | 77495 | UBX Domain-containing 1 | AI492365 |
| 16 | CTTGATTCCC | 0.9 | 6.4 | 0.0020 | 77266 | quiescin Q6 | |
| 17 | TGTGCTCGGG | 2.3 | 7.6 | 0.0091 | 76847 | KIAA0088 protein | |
| 18 | CAGTCTCTCA | 0.9 | 7.0 | 0.0010 | 76230 | ribosomal protein S10 | |
| 19 | TACGTACTGC | 0.0 | 3.5 | 0.0052 | 76086 | ESTs, Highly similar to small zinc | |
| | | | | | | finger-like protein [H. sapiens] | |
| 20 | GGCAAGCCCC | 9.9 | 40.1 | 0.0000 | 76067 | heat shock 27 kD protein 1 | AI540836 |
| 21 | CGGCTGAATT | 0.9 | 5.8 | 0.0043 | 75888 | phosphogluconate dehydrogenase | |
| 22 | GGCTGGGGGC | 19.4 | 38.3 | 0.0001 | 75721 | profilin 1 | AA903097 |
| 23 | TGCTGGGTGG* | 0.6 | 4.6 | 0.0056 | 754 | folylpolyglutamate synthase | |
| 24 | TCAGATCTTT | 39.7 | 70.9 | 0.0000 | 75344 | ribosomal protein S4, X-linked | AA954645 |
| 25 | AAGAGTTTTG | 0.5 | 5.8 | 0.0010 | 75313 | aido-keto reductase family 1, member B1 (aldose reduclase) | |
| 26 | ACAAATCCTT | 1.8 | 7.0 | 0.0087 | 752 | FK506-binding protein 1A (12 kD) | |
| 27 | AATATGTGGG | 5.4 | 12.8 | 0.0060 | 74649 | cytochrome c oxidase subunit Vlc | AI025170 |
| 28 | GGGTTTTTAT | 5,0 | 11.6 | 0.0100 | 74497 | nuclease sensitive element binding protein 1 | |
| 29 | TCTGCTTACA | 2.7 | 8.1 | 0.0093 | 74267 | ribosomal protein L15 | |
| 30 | TTTAAAAAAA* | 21.7 | 7.0 | 0.0003 | 74088 | early growth response 3 | |
| 31 | CAAACCATCC* | 1.4 | 5.8 | 0.0100 | 65114 | keratin 18 | |
| 32 | CAGACTTTTG* | 5.9 | 12.8 | 0.0100 | 63348 | DKFZP586M121 protein | |
| 33 | TTGGGGTTTC | 40.6 | 74.9 | 0.0000 | 62954 | ferritin, heavy polypeptide 1 | AA866040 |
| 34 | AAGCCTTGCT | 5.4 | 0.6 | 0.0098 | 6289 | growth factor receptor-bound protein 2 [No, now | AI039721 |
| | | | | | | described as small stress protein-like protein HSP22 | |
| 35 | AAGAAACCTT | 1.4 | 5.8 | 0.0100 | 5836 | ESTs, Highly similar to CGI-138 protein [H. sapiens] | |
| 36 | AAGGAAATGA | 4.5 | 0.0 | 0.0043 | 57929 | slit (Drosophila) homolog 3 | AI741785 |
| 37 | AACTAAAAAA* | 68.6 | 91.8 | 0.0010 | 55921 | glutamyl-prolyl-tRNA synthetase | |
| 38 | ATAATTCTTT | 73.1 | 102.8 | 0.0001 | 539 | ribosomal protein S29 | |
| 39 | GTAAAAAAAA* | 44.7 | 20.9 | 0.0002 | 460 | Activating Transcription Factor 3 | AA903193 |
| 40 | TTTTAAAAAT* | 7.7 | 1.7 | 0.0100 | 45033 | lacrimal proline rich protein | |
| 41 | CTCGAATAAA | 4.5 | 0.0 | 0.0043 | 34871 | KIAA0569 gene product | AI338485 |
| 42 | AACTAAAAAA* | 68.6 | 91.8 | 0.0010 | 3297 | ribosomal protein S27a | |
| 43 | GAGGCGATCA | 0.0 | 3.5 | 0.0052 | 30783 | ESTs, Weakly similar to eyelid [D. melanogaster] | |
| 44 | GTGGCTGAAA | 5.0 | 0.0 | 0.0027 | 29797 | ribosomal protein L10 | AI582446 |
| 45 | GTTTCCCCAA* | | | 0.0067 | 281434 | | |
| 46 | GGTGAAGACA | 8.6 | 1.2 | 0.0015 | 26951 | Human mRNA for KIAA0375 gene, complete cds | AI080611 |
| 47 | GCATTTAAAT* | 16.7 | 27.3 | 0.0065 | 250876 | eukaryotic translation elongation factor 1 beta 2 | |
| 48 | TTTTGTATTT | 5.9 | 0.6 | 0.0055 | 250705 | ESTs | |
| 49 | CAGACTTTTG* | 5.9 | 12.8 | 0.0100 | 250501 | TNF? elastin microfibril interface located protein | |
| 50 | GATCCCAACA | 0.5 | 4.6 | 0.0056 | 25 | ATP synthase, H+ transporting, mitochondrial | |
| | | | | | | F1 complex, beta polypeptide | |
| 51 | CTGTTGATTG | 16.3 | 26.7 | 0.0070 | 249495 | heterogeneous nuclear ribonucleoprotein A1 | |
| 52 | CACGCAATGC* | 0.5 | 5.8 | 0.0010 | 244 | amino-terminal enhancer of split | AI015996 |
| 53 | TGGTACACGT | 0.9 | 5.8 | 0.0043 | 242463 | keratin 8 | |
| 54 | TTTTAAAAAT* | 7.7 | 1.7 | 0.0100 | 240013 | catechol-O-methyltransferase | |
| 55 | ATTCTCCAGT | 48.8 | 72.6 | 0.0002 | 234518 | ribosomal protein L23 | |
| 56 | CCCTTAGCTT | 13.5 | 3.5 | 0.0011 | 233936 | myosin, light polypeptide, regulatory, non-sarcomeric (20 kD) | AI033904 |
| 57 | GTGGTGGGCG* | 0.9 | 7.0 | 0.0010 | 233694 | ESTs, Weakly similar to ZK1058.5 [C. elegans] | |
| 58 | GCGGGGTACC | 1.4 | 5.8 | 0.0100 | 227823 | pM5 protein | |
| 59 | GGAGAGTACA | 0.0 | 3.5 | 0.0052 | 225939 | sialyltransferase 9 (CMP-NeuAc:lactosylceramide | |
| | | | | | | alpha-2,3-sialyltransferase; GM3 synthase) | |
| 60 | TAAAATAAAA* | 5.0 | 0.0 | 0.0027 | 21254 | TRAF Interacting protein | |
| 61 | GTGGTGGGCG* | 0.9 | 7.0 | 0.0010 | 209741 | EST | |
| 62 | GTGGTGGGCG* | 0.9 | 7.0 | 0.0010 | 208985 | ESTs | |
| 63 | TCCAAATCGA | 6.8 | 1.2 | 0.0080 | 2064 | vimentin | |
| 64 | TAAAATAAAA* | 5.0 | 0.0 | 0.0027 | 204144 | ESTs, Moderately similar to tumor necrosis factor-alpha-Induced | |
| | | | | | | protein B12 [H. sapiens] | |
| 65 | TAAAATAAAA* | 5.0 | 0.0 | 0.0027 | 202218 | EST | |
| 66 | TTCAATAAAA* | 58.7 | 93.0 | 0.0000 | 2012 | transcobalamin I (vitamin B12 binding protein, R binder family) | AI738761 |
| 67 | TGCTGGGTGG* | 0.5 | 4.6 | 0.0056 | 198273 | NADH dehydrogenase (ubiquinone) 1 beta | |
| | | | | | | subcomplex, 8 (19 kD, ASHI) | |
| 68 | TAAAATAAAA* | 5.0 | 0.0 | 0.0027 | 190401 | ESTs, Weakly similar to predicted | |
| | | | | | | using Genefinder [C. elegans] | |
| 69 | AAGGTCGAGC | 1.8 | 7.0 | 0.0087 | 184582 | ribosomal protein L24 | |
| 70 | GCATAATAGG | 31.6 | 54.0 | 0.0001 | 184108 | ribosomal protein L21 | AI224420 |
| 71 | GTAAAAAAAA* | 44.7 | 20.9 | 0.0002 | 183842 | Ubiquitin B | AA988708 |
| 72 | TAATATTTTT | 11.7 | 2.9 | 0.0020 | 182485 | actinin, alpha 4 | |
| 73 | GAAAAATGGT | 48.3 | 87.7 | 0.0000 | 181357 | laminin receptor 1 (67 kD, ribosomal protein SA) | AI025931 |
| 74 | AGAACCTTAA | 5.0 | 0.0 | 0.0027 | 181244 | major histocompatibllity complex, class I, A | AI023950 |
| 75 | CTCATAAGGA | 49.2 | 70.9 | 0.0006 | 181165 | eukaryotic translation elongation factor 1 alpha 1 | |
| 76 | TAATTTTGGA | 9.0 | 17.4 | 0.0078 | 180152 | ESTs | |
| 77 | CAATAAATGT | 37.0 | 57.5 | 0.0004 | 179779 | ribosomal protein L37 | |
| 78 | TTCAATAAAA* | 58.7 | 93.0 | 0.0000 | 177592 | ribosomal protein, large, P1 | AA961386 |
| 79 | TTAAATAGCA | 6.8 | 0.6 | 0.0021 | 172928 | collagen, type I, alpha 1 | AA992596 |
| 80 | GGAGGAGAGC | 5.0 | 0.0 | 0.0027 | 172928 | collagen, type I, alpha 1 | |
| 81 | TGAAATTGTC | 4.5 | 0.0 | 0.0043 | 172928 | collagen, type I, alpha 1 | |
| 82 | AATGCAGGCA | 1.4 | 6.4 | 0.0067 | 172673 | S-adenosylhomocysteine hydrolase | AI051370 |
| 83 | TTCCGGTTCC | 9.0 | 1.7 | 0.0030 | 172609 | nucleobindin 1 | AI025019 |
| 84 | CACGCAATGC* | 0.5 | 5.8 | 0.0010 | 170683 | EST | AI015996 |
| 85 | TAGACTTATT | 0.5 | 4.6 | 0.0056 | 170197 | glutamic-oxaloacetic transaminase 2, | |
| | | | | | | mitochondrial (aspartate aminotransferase 2) | |
| 86 | TTCACAGTGG | 0.5 | 5.2 | 0.0025 | 169992 | protein phosphatase 3 (formerly 2B), regulatory | |
| | | | | | | subunit B (19 kD), alpha isoform (calclneurin B, type I) | |
| 87 | TGCACGTTTT | 37.5 | 61.0 | 0.0001 | 169793 | ribosomal protein L32 | AA922605 |
| 88 | GCAGCTCAGG | 2.3 | 7.6 | 0.0091 | 167137 | ESTs, Moderately similar to CATHEPSIN D | |
| | | | | | | PRECURSOR [H. sapiens] | |
| 89 | GGTGAGACAC | 2.7 | 8.7 | 0.0071 | 164280 | solute carrier family 25 (mitochondrlal carrier; adenine | |
| | | | | | | nucleotide translocator), member 6 | |
| 90 | TTTAAAAAAA* | 21.7 | 7.0 | 0.0003 | 155545 | 37 kDa leucine-rich repeat (LRR) protein | |
| 91 | ATGTCATCAA | 1.4 | 7.0 | 0.0030 | 152936 | adaptor-related protein complex 2, mu 1 subunit | |
| 92 | TAATAAAGGT | 45.1 | 62.2 | 0.0029 | 151604 | ribosomal protein S8 | AI312878 |
| 93 | TTAAAACAAA | 4.1 | 0.0 | 0.0070 | 150508 | ESTs | AI280093 |
| 94 | TGGCCTAAAA | 4.5 | 0.0 | 0.0043 | 1501 | syndecan 2 (heparan sulfate proteoglycan 1, | AI005403 |
| | | | | | | cell surface-associated, fibroglycan) | |
| 95 | GGTTAATTGA | 4.1 | 0.0 | 0.0070 | 14376 | | AI590977 |
| 96 | TTCAATAAAA* | 58.7 | 93.0 | 0.0000 | 141269 | ESTs | AA961386 |
| 97 | ATGTGAAGAA | 6.8 | 1.2 | 0.0080 | 13662 | Homo sapiens clone 25036 mRNA sequence | AI279223 |
| 98 | TTTAAAAAAA* | 21.7 | 7.0 | 0.0003 | 134533 | ESTs | |
| 99 | TTAGATAAGC* | 4.5 | 12.2 | 0.0036 | 134350 | ESTs | |
| 100 | GTGACAGAAG | 5.4 | 13.4 | 0.0034 | 129673 | eukaryotic translation Initiation factor 4A, Isoform 1 | |
| 101 | CTGGGTTAAT | 42.4 | 77.8 | 0.0000 | 126701 | ribosomal protein S19 | AA865047 |
| 102 | CAAACCATCC* | 1.4 | 5.8 | 0.0100 | 125170 | ESTs | |
| 103 | GGGACTGGGC* | 0.5 | 5.2 | 0.0025 | 122256 | ESTs | |
| 104 | GCATTTAAAT* | 16.7 | 27.3 | 0.0065 | 120979 | ESTs | |
| 105 | TAGGTTGTCT | 55.1 | 71.5 | 0.0057 | 119252 | tumor protein, translationally-controlled 1 | AA808956 |
| 106 | GACGTGTGGG | 0.0 | 4.6 | 0.0009 | 119192 | H2A histone family, member Z | |
| 107 | GGCTTTACCC | 9.0 | 20.9 | 0.0006 | 119140 | eukaryotic translation initiation factor 5A | |
| 108 | AACTAATACT | 4.5 | 12.8 | 0.0022 | 118724 | DR1-associated protein 1 (negative cofactor 2 alpha) | AI028098 |
| 109 | TAAAATAAAA* | 5.0 | 0.0 | 0.0027 | 118246 | ESTs | |
| 110 | GGGACTGGGC* | 0.5 | 5.2 | 0.0025 | 117848 | hemoglobin, epsilon 1 | |
| 111 | TAAAAACAAA | 5.4 | 0.6 | 0.0098 | 114599 | | AW029321, |
| | | | | | | | W1923 |
| 112 | GTTTCCCCAA* | 1.4 | 6.4 | 0.0067 | 112423 | Homo sapiens mRNA; cDNA DKFZp586I1420 | |
| | | | | | | (from clone DKFZp586I1420) | |
| 113 | GCAAAAAAAA | 67.3 | 31.4 | 0.0000 | 11221 | ESTs, Weakly similar to fos395541_1 [H. sapiens] | AI057027 |
| 114 | CCAGAACAGA | 37.5 | 55.8 | 0.0011 | 111222 | ribosomal protein L30 | |
| 115 | GTACGGAGAT | 0.5 | 4.6 | 0.0056 | 109225 | vascular cell adhesion molecule 1 | |
| 116 | GTTTCCCCAA* | 1.4 | 6.4 | 0.0067 | 107573 | sialyltransferase | |
| 117 | GGCCCCTCAC | 0.5 | 4.6 | 0.0056 | 106283 | Insulin-like growth factor binding protein 6 | |
| 118 | TTTAAAAAAA* | 21.7 | 7.0 | 0.0003 | 106204 | ESTs, Moderately similar to antigen containing epitope | |
| | | | | | | to monoclonal antibody MMS-85/12 [M. musculus] | |
| 119 | CCTCCCCCGT | 0.9 | 5.2 | 0.0092 | 10488 | Breakpoint cluster region protein, uterine | |
| | | | | | | leiomyoma, 1; barrier to autointegration factor | |
| 120 | AGCAGGGCTC | 0.0 | 5.2 | 0.0004 | 100623 | phospholipase C, beta 3, neighbor pseudogene | |
| 121 | AATTGCAAGC | 5.0 | 0.0 | 0.0027 | — | | U50523 |
| 122 | TTTAACGGCC | 43.8 | 12.8 | 0.0000 | — | | AA196553 |
| 123 | CATTGCCTTC | 5.9 | 0.6 | 0.0055 | — | | |
| 124 | GCTTGCTGCC | 4.5 | 0.0 | 0.0043 | — | | |
| 125 | AAAACATTCT | 67.7 | 32.5 | 0.0000 | — | | AI538076 |
| 126 | GCAGACATTG | 0.9 | 5.8 | 0.0043 | — | | |
| 127 | ACCCTTGGCC | 12.2 | 26.7 | 0.0003 | — | | |
| 128 | AGTAGGTGGC | 8.1 | 1.7 | 0.0068 | — | U5 snRNP-specific protein (220 kD), | |
| | | | | | | ortholog of S. cerevisiae Prp8p | |
| 129 | GCAAGCCAAC | 16.7 | 33.1 | 0.0002 | — | actinin, alpha 1 | Al669642 |
| 130 | TTAGCTTGTT | 4.1 | 0.0 | 0.0070 | — | | |
| 131 | GTAATAACTT | 4.1 | 0.0 | 0.0070 | — | | |
| 132 | CGCCGTCGGC | 0.0 | 3.5 | 0.0052 | — | | |
| 133 | ACTCTTTCAA | 0.5 | 4.6 | 0.0056 | — | | |
| 134 | TGCTACGAAA | 5.9 | 0.6 | 0.0055 | — | | |
| 135 | CCCCGGTACA | 7.2 | 1.2 | 0.0053 | — | | |
| 136 | TTATAAAAGA | 10.8 | 3.5 | 0.0098 | — | | |

TABLE 1
MYCN regulated genes (grouped by functional category)

| | | | Ratio | | | | | | |
|---|---|---|---|---|---|---|---|---|---|
| | | | ON: | P | | SHEP- | Unigene | | |
| No. | Tag Sequence | nd | OFF | value | SHEP-2 | 21N | Cluster | Unigene Description | Accession code |
| | | | | | | | | RIBOSOMAL PROTEINS | |
| 1 | GGCCGCGTTC | | 47.7 | 0.000 | 0.00 | 10.7 | 5174 | ribosomal protein S17 | AA876041, |
| | (SEQ ID NO: 1) | | | | | | | | AI564812, |
| 2 | CCAGTGGCCC | | 43.5 | 0.000 | 00.0 | 9.7 | 180920 | ribosomal protein S9 | AI064904, U14971 |
| | (SEQ ID NO: 2) | | | | | | | | |
| 3 | GCCGAGGAAG | | 38.3 | 0.000 | 0.4 | 17.2 | 82148 | ribosomal protein S12 | AA483128, |
| | (SEQ ID NO: 3) | | | | | | | | AA524764, |
| 4 | GGAGTGGACA | | 29.0 | 0.000 | 0.2 | 6.5 | 75458 | ribosomal protein L18 | L11566, AA513721, |
| | (SEQ ID NO: 4) | | | | | | | | |
| 5 | GTTAACGTCC | | 26.9 | 0.000 | 0.2 | 6.0 | 178391 | L-44-like ribosomal protein (L44L), Homo | W05120, AA181201, |
| | (SEQ ID NO: 5) | | | | | | | sapiens Bruton's tyrosine kinase (BTK) alpha- | |
| | | | | | | | | D-galactosidase A (GLA) | |
| 6 | GCTTTTAAGG | | 20.7 | 0.000 | 0.7 | 13.9 | 8102 | ribosomal protein S20 | AI741190, |
| | (SEQ ID NO: 6) | | | | | | | | AA064902, |
| 7 | GTGTTGCACA | | 16.6 | 0.000 | 0.4 | 7.4 | 165590 | ribosomal protein S13 | AI350382, |
| | (SEQ ID NO: 7) | | | | | | | | AI290903, |
| 8 | CCGTCCAAGG | | 16.6 | 0.000 | 0.9 | 14.8 | 80617 | ribosomal protein S16 | AI262556, |
| | (SEQ ID NO: 8) | | | | | | | | AA628078, |
| 9 | CTCGAGGAGG | | 12.4 | 0.006 | 0.2 | 2.8 | 3254 | ribosomal protein L23-like | U26596, Z49254, |
| | (SEQ ID NO: 9) | | | | | | | | |
| 10 | TCTCTTTTTC | · | 12.4 | 0.006 | 0.2 | 2.8 | 1948 | ribosomal protein S21 | AW196629 |
| | (SEQ ID NO: 10) | | | | | | | | |
| 11 | GTTCGTGCCA | | 12.4 | 0.000 | 0.7 | 8.3 | 179666 | ribosomal protein L35a | AW328435, |
| | (SEQ ID NO: 11) | | | | | | | | AW328458, |
| 12 | GTGTTAACCA | | 11.4 | 0.000 | 0.4 | 5.1 | 74267 | ribosomal protein L15 | AW327407, |
| | (SEQ ID NO: 12) | | | | | | | | AA669521, |
| 13 | CTCAACATCT | | 11.4 | 0.000 | 1.3 | 15.3 | 73742 | ribosomal protein, large, P0 | AA807754, |
| | (SEQ ID NO: 13) | | | | | | | | AI174682, |
| 14 | ATGGCTGGTA | | 11.0 | 0.000 | 3.1 | 34.3 | 182426 | ribosomal protein S2 | X17206, AA704039, |
| | (SEQ ID NO: 14) | | | | | | | | |
| 15 | AATCCTGTGG | | 9.9 | 0.000 | 2.2 | 22.3 | 178551 | ribosomal protein L8 | AW327732, |
| | (SEQ ID NO: 15) | | | | | | | | AI200056, |
| 16 | GACGACACGA | | 9.2 | 0.000 | 1.6 | 14.4 | 153177 | ribosomal protein S28 | AA229774, |
| | (SEQ ID NO: 16) | | | | | | | | AA421061, |
| 17 | TCTTCTCACA | · | 8.3 | 0.010 | 0.0 | 1.9 | 126400 | ESTs, Highly similar to 60S RIBOSOMAL | AA075869 |
| | (SEQ ID NO: 17) | | | | | | | PROTEIN L39 (H. sapiens) | |
| 18 | GCCTGTATGA | | 8.0 | 0.000 | 1.6 | 12.5 | 180450 | ribosomal protein S24 | AW410149, D51704, |
| | (SEQ ID NO: 18) | | | | | | | | |
| 19 | TGGTGTTGAG | | 7.7 | 0.000 | 2.2 | 17.2 | 75362 | ribosomal protein S18 | AA179543, |
| | (SEQ ID NO: 19) | | | | | | | | AW409851, |
| 20 | CCTCGGAAAA | | 7.4 | 0.000 | 1.6 | 11.6 | 2017 | ribosomal protein L38 | AA659304, |
| | (SEQ ID NO: 20) | | | | | | | | AA747113, |
| 21 | CCCATCCGAA | | 7.1 | 0.000 | 1.6 | 11.1 | 91379 | ribosomal protein L26 | AA084377, |
| | (SEQ ID NO: 21) | | | | | | | | AA111974, |
| 22 | GATGCTGCCA | | 6.6 | 0.000 | 1.1 | 7.4 | 99914 | ribosomal protein L22 | AA420829, |
| | (SEQ ID NO: 22) | | | | | | | | AA603500, |
| 23 | ATCAAGGGTG | | 6.0 | 0.000 | 1.8 | 10.7 | 157850 | ribosomal protein L9 | D14531, U09953, |
| | (SEQ ID NO: 23) | | | | | | | | |
| 24 | CGCCGCCGGC | | 5.9 | 0.000 | 3.1 | 18.6 | 182825 | ribosomal protein L35 | AA457581, |
| | (SEQ ID NO: 24) | | | | | | | | AA479946, |
| 25 | GGACCACTGA | | 5.9 | 0.000 | 2.7 | 15.8 | 119598 | ribosomal protein L3 | AA121930, |
| | (SEQ ID NO: 25) | | | | | | | | AW022183, |
| 26 | CAGCTCACTG | | 5.7 | 0.002 | 0.9 | 5.1 | 158675 | ribosomal protein L14 | AA857488, |
| | (SEQ ID NO: 26) | | | | | | | | AA186408, |
| 27 | GGGCTGGGGT | | 5.7 | 0.000 | 8.3 | 46.8 | 183698 | ribosomal protein L29 | U10248, U49083, |
| | (SEQ ID NO: 27) | | | | | | | | |
| 28 | AAGGTGGAGG | | 5.3 | 0.000 | 4.9 | 26.0 | 163593 | ribosomal protein L18a | AI719134, |
| | (SEQ ID NO: 28) | | | | | | | | AA047273, |
| 29 | CACAAACGGT | | 5.2 | 0.000 | 4.0 | 20.9 | 195453 | ribosomal protein S27 (metallopanstimulin 1) | AA525023, |
| | (SEQ ID NO: 29) | | | | | | | | AA555149, |
| 30 | CCCCAGCCAG | | 5.1 | 0.000 | 2.5 | 12.5 | 252259 | ribosomal protein S3 | AA706649, |
| | (SEQ ID NO: 30) | | | | | | | | AI792490, |
| 31 | TCGTCTTTAT | | 4.8 | 0.000 | 2.0 | 9.7 | 75538 | ribosomal protein S7 | M77233, AA513485, |
| | (SEQ ID NO: 31) | | | | | | | | |
| 32 | GTTCCCTGGC | | 4.7 | 0.000 | 2.7 | 12.5 | 177415 | ribosomal protein S30; Finkel-Biskis-Reilly | AA025263, |
| | (SEQ ID NO: 32) | | | | | | | murine sarcoma virus (FBR-MuSV) | AA055838, |
| | | | | | | | | ubiquitously expressed | |
| 33 | GAACACATCC | | 4.7 | 0.000 | 1.8 | 8.3 | 75879 | ribosomal protein L19 | AI219423, |
| | (SEQ ID NO: 33) | | | | | | | | AA075993, |
| 34 | CTGCTATACG | | 4.6 | 0.000 | 2.2 | 10.2 | 180946 | ribosomal protein L5 | W95798, AA491764, |
| | (SEQ ID NO: 34) | | | | | | | | |
| 35 | CCCATCGTCC | · | 4.3 | 0.000 | 21.5 | 91.4 | 151604 | ribosomal protein S8 | AI357743, |
| | (SEQ ID NO: 35) | | | | | | | | AW051118, |
| 36 | GCAGCCATCC | | 4.1 | 0.000 | 5.4 | 22.3 | 4437 | ribosomal protein L28 | AA225830, |
| | (SEQ ID NO: 36) | | | | | | | | AA480489, |
| 37 | AAGGAGATGG | | 4.0 | 0.000 | 3.1 | 12.5 | 184014 | ribosomal protein L31 | AA340384, |
| | (SEQ ID NO: 37) | | | | | | | | AA024502, |
| 38 | CCCGTCCGGA | | 4.0 | 0.000 | 7.6 | 30.1 | 180842 | ribosomal protein L13 | AI808685, |
| | (SEQ ID NO: 38) | | | | | | | | AI992115, |
| 39 | CTGTTGGTGA | | 3.9 | 0.000 | 4.0 | 15.8 | 3463 | ribosomal protein S23 | AW020385, |
| | (SEQ ID NO: 39) | | | | | | | | AW022474, |
| 40 | AGGGCTTCCA | | 3.8 | 0.000 | 5.1 | 19.5 | 29797 | ribosomal protein L10 | AA244126, |
| | (SEQ ID NO: 40) | | | | | | | | AA508386, |
| 41 | TAAGGAGCTG | | 3.8 | 0.000 | 3.8 | 14.4 | 77904 | ribosomal protein S26 | AW022428, X69654, |
| | (SEQ ID NO: 41) | | | | | | | | |
| 42 | GTGAAGGCAG | | 3.7 | 0.000 | 3.4 | 12.5 | 77039 | ribosomal protein S3A | AA070817, |
| | (SEQ ID NO: 42) | | | | | | | | AA074191, |
| 43 | CGCCGGAACA | | 3.4 | 0.000 | 3.8 | 13.0 | 286 | ribosomal protein L4 | AL119264, |
| | (SEQ ID NO: 43) | | | | | | | | AW021149, |
| 44 | AGGCTACGGA | | 3.4 | 0.000 | 8.7 | 29.7 | 119122 | ribosomal protein L13a | AA190354, |
| | (SEQ ID NO: 44) | | | | | | | | AW328422, |
| 45 | CCTTCGAGAT | | 3.3 | 0.000 | 4.0 | 13.5 | 76194 | ribosomal protein S5 | AI971757, |
| | (SEQ ID NO: 45) | | | | | | | | AA057826, |
| 46 | AAGACAGTGG | | 3.3 | 0.000 | 9.2 | 30.1 | 184109 | ribosomal protein L37a | AA353060, |
| | (SEQ ID NO: 46) | | | | | | | | AW020311, |
| 47 | TTACCATATC | | 3.3 | 0.000 | 6.9 | 22.7 | 177461 | ribosomal protein L39 | AW023657, T40220, |
| | (SEQ ID NO: 47) | | | | | | | | |
| 48 | TGTGCTAAAT | | 3.2 | 0.000 | 5.1 | 16.7 | 250695 | ribosomal protein L34 | AW020881, N85940, |
| | (SEQ ID NO: 48) | | | | | | | | |
| 49 | GCCGTGTCCG | | 3.2 | 0.001 | 2.9 | 9.3 | 241507 | ribosomal protein S6 | AA148286, |
| | (SEQ ID NO: 49) | | | | | | | | AA167421, |
| 50 | GGCAAGAAGA | | 3.0 | 0.000 | 3.8 | 11.6 | 111611 | ribosomal protein L27 | AA531226, |
| | (SEQ ID NO: 50) | | | | | | | | AA885969, |
| 51 | ACATCATCGA | | 3.0 | 0.000 | 7.4 | 22.3 | 182979 | ribosomal protein L12 | L06505, AA484253, |
| | (SEQ ID NO: 51) | | | | | | | | |
| 52 | GGATTTGGCC | | 2.9 | 0.000 | 16.8 | 48.2 | 251247 | ribosomal protein, large P2 | AA037465, |
| | (SEQ ID NO: 52) | | | | | | | | AA064832, |
| 53 | CGCTGGTTCC | | 2.8 | 0.000 | 8.5 | 24.1 | 179943 | ribosomal protein L11 | AA226392, |
| | (SEQ ID NO: 53) | | | | | | | | AA230322, |
| 54 | TACAAGAGGA | | 2.8 | 0.006 | 2.7 | 7.4 | 174131 | ribosomal protein L6 | AI042168, D17554 |
| | (SEQ ID NO: 54) | | | | | | | | |
| 55 | TTGGTCCTCT | | 2.7 | 0.000 | 18.4 | 50.1 | 108124 | ribosomal protein L41 | Z12962, AA045319, |
| | (SEQ ID NO: 55) | | | | | | | | |
| 56 | CTCCTCACCT | | 2.6 | 0.001 | 4.9 | 13.0 | 119122 | ribosomal protein L13a | AW242158, |
| | (SEQ ID NO: 56) | | | | | | | | AW245640, |
| 57 | AATAGGTCCA | | 2.5 | 0.000 | 9.2 | 23.2 | 113029 | ribosomal protein S25 | AA228780, |
| | (SEQ ID NO: 57) | | | | | | | | AA229897, |
| 58 | GAGGGAGTTT | | 2.5 | 0.000 | 18.6 | 46.8 | 76064 | ribosomal protein L27a | AA228189, |
| | (SEQ ID NO: 58) | | | | | | | | AA229949, |
| 59 | AAGAAGATAG | | 2.5 | 0.002 | 4.3 | 10.7 | 184776 | ribosomal protein L23a | AA040728, |
| | (SEQ ID NO: 59) | | | | | | | | AA088884, |
| 60 | ATTATTTTTC | | 2.5 | 0.006 | 3.6 | 8.8 | 153 | ribosomal protein L7 | AA138446, |
| | (SEQ ID NO: 60) | | | | | | | | AW020191, |
| 61 | ACTCCAAAAA | | 2.2 | 0.000 | 10.3 | 22.7 | 133230 | ribosomal protein S15 | AA079663, |
| | (SEQ ID NO: 61) | | | | | | | | AA151459, |
| | SECTION 2 | | | | | | | PROTEIN SYNTHESIS | |
| 62 | CTGGCGAGCG | | 18.6 | 0.000 | 0.2 | 4.2 | 174070 | ubiquitin carrier protein | AA211097, |
| | (SEQ ID NO: 62) | | | | | | | | AA283711, |
| 63 | GAGCGGGATG | | 16.6 | 0.001 | 0.2 | 3.7 | 77060 | proteasome (prosome, macropain) subunit | D29012, X61971, |
| | (SEQ ID NO: 63) | | | | | | | beta type, 6 | |
| 64 | GCATAGGCTG | | 12.4 | 0.000 | 0.4 | 5.6 | 12084 | Tu translation elongation factor, | AL037768, L38995, |
| | (SEQ ID NO: 64) | | | | | | | mitochondrial | |
| 65 | GGCTCCCACT | | 11.7 | 0.000 | 0.7 | 7.9 | 74335 | heat shock 90 kD protein 1, beta | AW023752, |
| | (SEQ ID NO: 65) | | | | | | | | AA034511, |
| 66 | GCCCAGCTGG | | 11.4 | 0.000 | 0.4 | 5.1 | 223241 | eukaryotic translation elongation factor 1 delta | Z21507, AA489523, |
| | (SEQ ID NO: 66) | | | | | | | (guanine nucleotide exchange protein) | |
| 67 | TGAAATAAAC | · | 10.4 | 0.004 | 0.0 | 2.3 | 173205 | nucleophosmin (nucleolar phosphoprotein | AI184619 |
| | (SEQ ID NO: 67) | | | | | | | B23, numatrin) | |
| 68 | TACCAGTGTA | | 10.4 | 0.004 | 0.0 | 2.3 | 79037 | heat shock 60 kD protein 1 (chaperonin) | AW021205, |
| | (SEQ ID NO: 68) | | | | | | | | AW103323, |
| 69 | TGTGTTGAGA | | 9.4 | 0.000 | 5.6 | 52.4 | 181165 | eukaryotic translation elongation factor 1 | AA630271, |
| | (SEQ ID NO: 69) | | | | | | | alpha 1 | AA668532, |
| 70 | TGGGCAAAGC | | 9.0 | 0.000 | 2.0 | 18.1 | 2188 | eukaryotic translation elongation factor 1 | AA038923, |
| | (SEQ ID NO: 70) | | | | | | | gamma | AA190762, |
| 71 | ACATCCTCAC | | 8.3 | 0.010 | 0.0 | 1.9 | 18700 | proteasome (prosome, macropain) 26S | AA024838, |
| | (SEQ ID NO: 71) | | | | | | | subunit, non-ATPase, 13 | AA149127, |
| 72 | TAAAATTTGT | · | 8.3 | 0.010 | 0.0 | 1.9 | 93379 | eukaryotic translation initiation factor 4B | AA054750, |
| | (SEQ ID NO: 72) | | | | | | | | AA133563, |
| 73 | CAGTCTAAAA | | 8.3 | 0.003 | 0.4 | 3.7 | 76118 | ubiquitin carboxyl-terminal esterase L1 | X04741, AA029783, |
| | (SEQ ID NO: 73) | | | | | | | (ubiquitin thiolesterase) | |
| 74 | GAAGGCATCC | | 8.3 | 0.010 | 0.0 | 1.9 | 250758 | proteasome (prosome, macropain) 28S | F32284, AA130329, |
| | (SEQ ID NO: 74) | | | | | | | subunit, ATPase, 3 | |
| 75 | TGGCTAGTGT | | 6.9 | 0.001 | 0.7 | 4.6 | 118865 | proteasome (prosome, macropain) subunit, | AA804284, |
| | (SEQ ID NO: 75) | | | | | | | beta type, 7 | AA829376, |
| 76 | CAGATCTTTG | | 5.8 | 0.000 | 1.1 | 6.5 | 119502 | ubiquitin A-52 residue ribosomal protein | AF075321, |
| | (SEQ ID NO: 76) | | | | | | | fusion product 1 | AI110823, |
| 77 | TCACAAGCAA | | 4.4 | 0.001 | 1.6 | 7.0 | 146763 | nascent-polypeptide-associated complex alpha | AF054187, X80909, |
| | (SEQ ID NO: 77) | | | | | | | polypeptide | |
| 78 | AACTCTTGAA | | 3.6 | 0.006 | 1.3 | 5.1 | 58189 | eukaryotic translation initiation factor 3, | U54559, AA024720, |
| | (SEQ ID NO: 78) | | | | | | | subunit 3 (gamma, 40 kD) | |
| 79 | AGCACCTCCA | | 3.5 | 0.000 | 9.0 | 31.5 | 75309 | eukaryotic translation elongation factor 2 | AA229607, |
| | (SEQ ID NO: 79) | | | | | | | | AA533837, |
| 80 | AGCCCTACAA | · | 3.4 | 0.000 | 15.4 | 52.9 | 180532 | heat shock 90 kD protein 1, alpha | AW088888, |
| | (SEQ ID NO: 80) | | | | | | | | AW128905, |
| 81 | CGCCGCGGTG | | 2.9 | 0.004 | 2.7 | 7.9 | 4835 | eukaryotic translation initiation factor 3, | AA573953, |
| | (SEQ ID NO: 81) | | | | | | | subunit 8 (110 kD) | AA778265, |
| 82 | TTCATACACC | · | 2.6 | 0.000 | 31.3 | 82.8 | 180532 | heat shock 90 kD protein 1, alpha | AW080984 |
| | (SEQ ID NO: 82) | | | | | | | | |
| 83 | TGAAATAAAA | | 2.5 | 0.000 | 10.1 | 25.5 | 173205 | nucleophosmin (nucleolar phosphoprotein | AI926288, |
| | (SEQ ID NO: 83) | | | | | | | B23, numatrin) | AW166350, |
| 84 | ATCAGTGGCT | | 2.5 | 0.009 | 3.1 | 7.9 | 89545 | proteasome (prosome, macropain) subunit, | R25997, R68878, |
| | (SEQ ID NO: 84) | | | | | | | beta type, 4 | |
| 85 | CCCTGATTTT | | −7.1 | 0.000 | 9.8 | 1.4 | 183684 | eukaryotic translation initiation factor 4 | U73824, U76111, |
| | (SEQ ID NO: 85) | | | | | | | gamma, 2 | |
| 86 | AAGAGGTTTG | | −9.2 | 0.001 | 4.3 | 0.0 | 74368 | transmembrane protein (63 kD), endoplasmic | X69910, AI131495, |
| | (SEQ ID NO: 86) | | | | | | | reticulum/Golgi intermediate compartment | |
| 87 | GTTTTTGCTT | | 2.4 | 0.075 | 1.6 | 3.7 | 79110 | Nucleolin | AA133588, |
| | (SEQ ID NO: 87) | | | | | | | | AA135423, A |
| 88 | TACAAAACCA | | 2.1 | 0.923 | 0.9 | 1.9 | 79110 | Nucleolin | AA088423, |
| | (SEQ ID NO: 88) | | | | | | | | AA284953, A |
| | SECTION 3. | | | | | | | GENES INVOLVED IN METASTASIS | |
| 89 | AATAGAAATT | | 14.5 | 0.000 | 0.0 | 3.2 | 313 | secreted phosphoprotein 1 (osteopontin, bone | AW021049, |
| | (SEQ ID NO: 89) | | | | | | | sialoprotein 1, early T-lymphocyte activation | AA021512, |
| | | | | | | | | 1) | |
| 90 | ATGCTCCCTG | | 14.5 | 0.000 | 0.0 | 3.2 | 79339 | lectin, galactoside-binding, soluble, 3 binding | L13210, X79089 |
| | (SEQ ID NO: 90) | | | | | | | protein (galectin 6 binding protein) | |
| 91 | ACAGGGTGAC | | 14.5 | 0.000 | 0.0 | 3.2 | 174050 | endothelial differentiation-related factor 1 | AA975055, |
| | (SEQ ID NO: 91) | | | | | | | | AA992919, |
| 92 | AACGCGGCCA | | 12.8 | 0.000 | 1.1 | 14.4 | 73798 | macrophage migration inhibitory factor | AA523321, |
| | (SEQ ID NO: 92) | | | | | | | (glycosylation-inhibiting factor) | AA927284 |
| 93 | AAGAAAGGAG | | 11.4 | 0.000 | 0.9 | 10.2 | 202097 | procollagen C-endopeptidase enhancer | L33799 |
| | (SEQ ID NO: 93) | | | | | | | | |
| 94 | GTAAGTCTCA | | 8.3 | 0.010 | 0.0 | 1.9 | 211584 | neurofilament, light polypeptide (68 kD) | AW022557, |
| | (SEQ ID NO: 94) | | | | | | | | AA330627, |
| 95 | GCCGATCCTC | | 8.3 | 0.010 | 0.0 | 1.9 | 24930 | tubulin-specific chaperone a | AF038952 |
| | (SEQ ID NO: 95) | | | | | | | | |
| 96 | GGCTCCTGGC | | 8.3 | 0.010 | 0.0 | 1.9 | 5215 | integrin beta 4 binding protein | AF022229, |
| | (SEQ ID NO: 96) | | | | | | | | AF047433, |
| 97 | GCCGGGTGGG | | 7.6 | 0.001 | 0.7 | 5.1 | 74631 | basigin | D45131, L10240, |
| | (SEQ ID NO: 97) | | | | | | | | |
| 98 | GGGGAAATCG | | 5.9 | 0.000 | 3.8 | 22.3 | 76293 | thymosin, beta 10 | AA147288, |
| | (SEQ ID NO: 98) | | | | | | | | AA155816, |
| 99 | GCCCCCAATA | | −1.7 | 0.007 | 20.1 | 11.6 | 227751 | lectin, galactoside-binding, soluble, 1 | J04456, X14829, |
| | (SEQ ID NO: 99) | | | | | | | (galectin 1) | |
| 100 | ACCAAAAACC | | −1.8 | 0.003 | 21.3 | 11.6 | 172928 | collagen, type I, alpha 1 | AA454809, |
| | (SEQ ID NO: 100) | | | | | | | | AA454820, |
| 101 | GTTGTGGTTA | | −2.2 | 0.000 | 30.0 | 13.9 | 75415 | beta-2-microglobulin | AB021288, |
| | (SEQ ID NO: 101) | | | | | | | | AA897072, |
| 102 | AACTGCTTCA | | −2.5 | 0.002 | 12.8 | 5.1 | 11538 | actin related protein 2/3 complex, subunit fB | AA031434, |
| | (SEQ ID NO: 102) | | | | | | | (41 kD) | AA045773, |
| 103 | TCTCTGATGC | | −2.7 | 0.001 | 12.5 | 4.6 | 6441 | tissue inhibitor of metalloproteinase 2 | AL110197, |
| | (SEQ ID NO: 103) | | | | | | | | AL134962, |
| 104 | TGTACCTGTA | | −2.8 | 0.000 | 14.1 | 5.1 | 169476 | tubulin, alpha, ubiquitous | AA018607, |
| | (SEQ ID NO: 104) | | | | | | | | AA018627, |
| 105 | GCTTTATTTG | | −2.9 | 0.000 | 15.9 | 5.6 | 180952 | actin, beta | AA419273, |
| | (SEQ ID NO: 105) | | | | | | | | AA528145, |
| 106 | TCCTGTAAAG | | −3.1 | 0.007 | 7.2 | 2.3 | 74034 | caveolin 1, caveolae protein, 22 kD | AA034380, |
| | (SEQ ID NO: 106) | | | | | | | | AA044994, |
| 107 | TGCTAAAAAA | | −4.7 | 0.000 | 17.5 | 3.7 | 146550 | myosin, heavy polypeptide 9, non-muscle | AA041529, |
| | (SEQ ID NO: 107) | | | | | | | | AA070528, |
| 108 | TGGAAATGAC | | −4.9 | 0.000 | 42.8 | 8.8 | 172928 | collagen, type I, alpha 1 | AA853342, |
| | (SEQ ID NO: 108) | | | | | | | | AA436411, |
| 109 | TGGAAATGCC | | −5.0 | 0.001 | 6.9 | 1.4 | 172928 | collagen, type I, alpha 1 | AI190987, |
| | (SEQ ID NO: 109) | | | | | | | | AI339782, |
| 110 | GTTTTTTTTA | · | −5.5 | 0.004 | 5.1 | 0.9 | 179573 | collagen, type I, alpha 2 | AI825593 |
| | (SEQ ID NO: 110) | | | | | | | | |
| 111 | ACAGGCTACG | | −6.0 | 0.002 | 5.6 | 0.9 | 75777 | transgelin | AI188763, |
| | (SEQ ID NO: 111) | | | | | | | | AI540294, |
| 112 | TAAAAATGTT | | −6.3 | 0.000 | 17.5 | 2.8 | 82085 | plasminogen activator inhibitor, type I | M14083, AA040151, |
| | (SEQ ID NO: 112) | | | | | | | | |
| 113 | GTTTCTAATA | | −6.3 | 0.006 | 2.9 | 0.0 | 239298 | microtubule-associated protein 4 | AA043400, |
| | (SEQ ID NO: 113) | | | | | | | | AA086286, |
| 114 | TTAAAGATTT | | −7.1 | 0.000 | 13.2 | 1.9 | 77899 | tropomyosin 1 (alpha) | AA026364, |
| | (SEQ ID NO: 114) | | | | | | | | AA036782, |
| 115 | GACCGCAGGA | | −7.2 | 0.003 | 3.4 | 0.0 | 119129 | collagen, type IV, alpha 1 | X03883, AW020005, |
| | (SEQ ID NO: 115) | | | | | | | | |
| 116 | TAATCCTCAA | | −8.2 | 0.000 | 7.6 | 0.9 | 78409 | collagen, type XVIII, alpha 1 | AA009957, |
| | (SEQ ID NO: 116) | | | | | | | | AA099281, |
| 117 | TGTAGAAAAA | | −9.2 | 0.004 | 4.3 | 0.5 | 119076 | tubulin, beta polypeptide | AI308800, |
| | (SEQ ID NO: 117) | | | | | | | | AI623884, |
| 118 | TTAGTGTCGT | | −10.6 | 0.002 | 4.9 | 0.5 | 111779 | secreted protein, acidic, cysteine-rich | AW020585, |
| | (SEQ ID NO: 118) | | | | | | | (osteonectin) | AW022769, |
| 119 | GCCCCAATAA | · | −11.1 | 0.000 | 5.1 | 0.0 | 227751 | lectin, galactoside-binding, soluble, 1 | AA095630 |
| | (SEQ ID NO: 119) | | | | | | | (galectin I) | |
| 120 | AAAGTCATTG | · | −11.1 | 0.001 | 5.1 | 0.5 | 77899 | tropomyosin 1 (alpha) | M19713 |
| | (SEQ ID NO: 120) | | | | | | | | |
| 121 | TGCAATATGC | | −12.5 | 0.000 | 5.8 | 0.0 | 750 | fibrillin 1 (Marfan syndrome) | L13923, X63556, |
| | (SEQ ID NO: 121) | | | | | | | | |
| 122 | ATCTTGTTAC | | −14.5 | 0.000 | 6.7 | 0.5 | 116162 | fibronectin 1 | AW020421, |
| | (SEQ ID NO: 122) | | | | | | | | AW020447, |
| 123 | AAAAAGCTGC | | −15.9 | 0.000 | 7.4 | 0.0 | 111779 | secreted protein, acidic, cysteine-rich | AA987875, |
| | (SEQ ID NO: 123) | | | | | | | (osteonectin) | AI755199, |
| 124 | AAAATATTTT | | −18.8 | 0.000 | 8.7 | 0.0 | 119000 | actinin, alpha 1 | X55187, AA024895, |
| | (SEQ ID NO: 124) | | | | | | | | |
| 125 | ATGTGAAGAG | | −19.9 | 0.000 | 268.2 | 13.5 | 111779 | secreted protein, acidic, cysteine-rich | AA228362, |
| | (SEQ ID NO: 125) | | | | | | | (osteonectin) | AA852763, |
| 126 | TGGCCTAATA | | −27.5 | 0.000 | 12.8 | 0.0 | 1501 | syndecan 2 (heparin sulfate proteoglycan 1, | AA873519, |
| | (SEQ ID NO: 126) | | | | | | | cell surface-associated, fibroglycan) | AI279414, |
| 127 | TTTGCACCTT | | −37.6 | 0.000 | 17.5 | 0.5 | 75511 | connective tissue growth factor | AW021964, U14750, |
| | (SEQ ID NO: 127) | | | | | | | | |
| | SECTION 4. | | | | | | | GLYCOLYSIS ENZYMES | |
| 128 | TAGCTTCTTC | | 14.5 | 0.000 | 0.0 | 3.2 | 76392 | aldehyde dehydrogenase 1, soluble | AA911018, |
| | (SEQ ID NO: 128) | | | | | | | | AI150648, |
| 129 | ACCTTGTGCC | | 10.4 | 0.004 | 0.0 | 2.3 | 878 | sorbitol dehydrogenase | AA570172, |
| | (SEQ ID NO: 129) | | | | | | | | AA570189, |
| 130 | TCTGCTTGTC | | 10.4 | 0.004 | 0.0 | 2.3 | 76392 | aldehyde dehydrogenase 1, soluble | H79748, H05271, |
| | (SEQ ID NO: 130) | | | | | | | | |
| 131 | TGACTGAAGC | | 10.4 | 0.004 | 0.0 | 2.3 | 3343 | 3-phosphoglycerate dehydrogenase | AA742550, |
| | (SEQ ID NO: 131) | | | | | | | | AA113268, |
| 132 | TGGCCCCACC | | 7.5 | 0.000 | 1.1 | 8.3 | 198281 | pyruvate kinase, muscle | AA766601, |
| | (SEQ ID NO: 132) | | | | | | | | AA768285, |
| 133 | GCGACCGTCA | | 7.3 | 0.000 | 0.9 | 6.5 | 183760 | aldolase A, fructose-bisphosphate | AA169762, |
| | (SEQ ID NO: 133) | | | | | | | | AA557193, |
| 134 | TACCATCAAT | | 4.7 | 0.000 | 5.8 | 27.4 | 195188 | glyceraldehyde-3-phosphate dehydrogenase | AA226658, |
| | (SEQ ID NO: 134) | | | | | | | | AA522734, |
| 135 | TGAGGGAATA | | 4.4 | 0.000 | 2.2 | 9.7 | 83848 | triosphosphate isomerase 1 | AA587096, |
| | (SEQ ID NO: 135) | | | | | | | | AA587188, |
| | | | | | | | | MITOCHONDRIAL FUNCTION | |
| | SECTION 5. | | | | | | | PROTEINS | |
| 136 | GAATCGGTTA | | 22.8 | 0.000 | 0.2 | 5.1 | 80595 | NADH dehydrogenase (ubiquinone) Fe-S | AF047434, |
| | (SEQ ID NO: 136) | | | | | | | (15 kD) (NDUFB4) | AA457600, |
| 137 | GGGGGTCACC | | 18.6 | 0.000 | 0.0 | 4.2 | 80986 | ATP synthase, H+ transporting, mitochondrial | AA659764, |
| | (SEQ ID NO: 137) | | | | | | | F0 complex, subunit c (subunit 9), isoform 1 | AA866065, |
| 138 | AAGGAGTTTG | | 14.5 | 0.000 | 0.0 | 3.2 | 661 | NADH dehydrogenase (ubiquinone) 1 beta | AA493442, |
| | (SEQ ID NO: 138) | | | | | | | subcomplex, 7 (18 kD, B18) | AA832435, |
| 139 | AGGTCCTAGC | | 14.5 | 0.000 | 0.4 | 6.5 | 226795 | glutathione S-transferase pi | F34719, U30897, |
| | (SEQ ID NO: 139) | | | | | | | | |
| 140 | CTTAGAGCCC | | 12.4 | 0.006 | 0.2 | 2.8 | 211929 | thioredoxin, mitochondrial | AI680239 |
| | (SEQ ID NO: 140) | | | | | | | | |
| 141 | TTCTGGCTGC | | 12.4 | 0.006 | 0.2 | 2.8 | 119251 | ubiquinol-cytochrome c reductase core protein 1 | F28971, AI005342, |
| | (SEQ ID NO: 141) | | | | | | | | |
| 142 | GTGGTACAGG | | 7.3 | 0.007 | 0.4 | 3.2 | 31731 | EST's, Highly similar to PUTATIVE | AA745952, |
| | (SEQ ID NO: 142) | | | | | | | PEROXISOMAL ANTIOXIDANT | AA829707, |
| | | | | | | | | ENZYME (H. sapiens) | |
| 143 | GTGACAACAC | | 5.2 | 0.003 | 0.9 | 4.6 | 149155 | voltage-dependent anion channel 1 | AA907238, |
| | (SEQ ID NO: 143) | | | | | | | | AI075271, |
| 144 | GCCTGCTGGG | | 4.8 | 0.000 | 2.0 | 9.7 | 2706 | glutathione peroxidase 4 phospholipid | X71973, AA743017, |
| | (SEQ ID NO: 144) | | | | | | | hydroperoxidase) | |
| 145 | GGGGACTGAA | | 4.6 | 0.003 | 1.1 | 5.1 | 3709 | low molecular mass ubiquinone-binding | AA010879, |
| | (SEQ ID NO: 145) | | | | | | | protein (9.5 kD) | AA025093, |
| 146 | CCCATCGTCC | · | 4.3 | 0.000 | 21.5 | 91.4 | mito | cytochrome c oxidase II (Welle et al., 1999), | AA069405, |
| | (SEQ ID NO: 146) | | | | | | | Tag matches mitochondrial sequences | AA074137 |
| 147 | TGATTTCACT | · | 4.1 | 0.000 | 4.0 | 18.7 | mito | cytochrome c oxidase III (Welle et al., 1999), | AA757623 |
| | (SEQ ID NO: 147) | | | | | | | Tag matches mitochondrial sequences | |
| 148 | TGAAGGAGCC | | 4.1 | 0.007 | 1.1 | 4.6 | 89399 | ATP synthase, H+ transporting, mitochondrial | F27013, D13119, |
| | (SEQ ID NO: 148) | | | | | | | F0 complex, subunit c (subunit 9), isoform 2 | |
| 149 | GTGACCTCCT | | 4.1 | 0.003 | 1.3 | 5.6 | 81097 | cytochrome c oxidase subunit VIII | F28051, F28676, |
| | (SEQ ID NO: 149) | | | | | | | | |
| 150 | AGCCCTACAA | · | 3.4 | 0.000 | 15.4 | 52.9 | mito | NADH dehydrogenase 3 (Welle et al., 1999), | AA577697, |
| | (SEQ ID NO: 150) | | | | | | | Tag matches mitochondrial sequences | AI022799 |
| 151 | TTCATACACC | · | 2.6 | 0.000 | 31.3 | 82.6 | mito | NADH dehydrogenase 4/4L (Welle et al., | AA971178 |
| | (SEQ ID NO: 151) | | | | | | | 1999), Tag matches mitochondrial sequences | |
| 152 | CACCTAATTG | · | 2.3 | 0.000 | 34.2 | 79.3 | mito | ATPase 6/8 (Welle et al., 1999), Tag matches | AA031407 |
| | (SEQ ID NO: 152) | | | | | | | mitochondrial sequences | |
| 153 | GAGAGCTCCC | | −2.6 | 0.008 | 7.8 | 2.8 | 169919 | electron-transfer-flavoprotein, alpha | AI364921 |
| | (SEQ ID NO: 153) | | | | | | | polypeptide (glutaric aciduria II) | |
| 154 | GTAAGATTAG | · | −5.8 | 0.000 | 8.1 | 1.4 | 5417 | oxygen regulated protein (150 kD) | AA828743 |
| | (SEQ ID NO: 154) | | | | | | | | |
| 155 | GCCCCAATAA | · | −11.1 | 0.000 | 5.1 | 0.0 | 173554 | ubiquinol-cytochrome c reductase core protein | AA425586 |
| | (SEQ ID NO: 155) | | | | | | | II | |
| | SECTION 6. | | | | | | | OTHER GENES | |
| 156 | CCCCCTGGAT | | 21.8 | 0.000 | 0.4 | 9.7 | N/A | calcyclin | AI832624, |
| | (SEQ ID NO: 156) | | | | | | | | |
| 157 | GTGCGCTAGG | | 18.6 | 0.000 | 0.2 | 4.2 | 9408 | ESTs, classified Into serine/threonine kinase, | AA420761, |
| | (SEQ ID NO: 157) | | | | | | | Highly similar to The KIAA0151gene product | AA492367, |
| | | | | | | | | is | |
| 158 | GAGTGGGGGC | | 18.6 | 0.000 | 0.2 | 4.2 | 14089 | ESTs, Weakly similar to LYSOSOMAL | AA362125, |
| | (SEQ ID NO: 158) | | | | | | | PRO-X CARBOXYPEPTIDASE | AA418395, |
| | | | | | | | | PRECURSOR (H. sapiens) | |
| 159 | TTTCCTTCCT | · | 16.6 | 0.001 | 0.2 | 3.7 | 104143 | clathrin, light polypeptide (Lca) | AA574409, |
| | (SEQ ID NO: 159) | | | | | | | | AA737504, |
| 160 | ATAAGATACA | | 16.6 | 0.000 | 0.0 | 3.7 | 199026 | RAS p21 protein activator (GTPase activating | X69399 |
| | (SEQ ID NO: 160) | | | | | | | protein) 3 (Ins(1,3,4,5)P4-binding protein | |
| 161 | AACGCTGCCT | | 14.5 | 0.000 | 0.0 | 3.2 | 28914 | adenine phosphoribosyltransferase | AA444001, |
| | (SEQ ID NO: 161) | | | | | | | | AA535523 |
| 162 | ATTTGTCCCA | | 12.4 | 0.000 | 0.4 | 5.6 | 139800 | high-mobility group (nonhistone | AA071304, |
| | (SEQ ID NO: 162) | | | | | | | chromosomal) protein Isoforms I and Y | AA074087, |
| 163 | TCAGACGCAG | | 12.4 | 0.006 | 0.2 | 2.8 | 182371 | prothymosin, alpha (gene sequence 28) | AW129309 |
| | (SEQ ID NO: 163) | | | | | | | | |
| 164 | TCTCTTTTTC | · | 12.4 | 0.006 | 0.2 | 2.8 | 119529 | epididymal secretory protein (19.5 kD) | AA025798, |
| | (SEQ ID NO: 164) | | | | | | | | AA039545, |
| 165 | TTTCAGGGGA | · | 12.4 | 0.006 | 0.2 | 2.8 | 2853 | poly(rC)-binding protein 1 | AI312897 |
| | (SEQ ID NO: 165) | | | | | | | | |
| 166 | CGGCCCAACG | | 12.4 | 0.001 | 0.0 | 2.8 | 20521 | HMTI (hnRNP methytransferase, S, | AA031375, |
| | (SEQ ID NO: 166) | | | | | | | cerevisiae)-like 2 | AA187773, |
| 167 | TTCTCCCGCT | | 12.4 | 0.001 | 0.0 | 2.8 | 118126 | protective protein for beta-galactosidase | M22960, AI752680, |
| | (SEQ ID NO: 167) | | | | | | | (galactosialidosis) | |
| 168 | GGGGGACGGC | | 12.4 | 0.001 | 0.0 | 2.8 | 21346 | ESTs, Weakly similar to F42C5.7 gene | AA015864, |
| | (SEQ ID NO: 168) | | | | | | | product (C. elegans) | AA026165, |
| 169 | GGCCCTGAGC | | 11.4 | 0.000 | 0.4 | 5.1 | 71618 | polymerase (RNA) II (DNA directed) | AA570105, |
| | (SEQ ID NO: 169) | | | | | | | polypeptide L (7.6 kD), Human RNA | AA562657, |
| | | | | | | | | polymerase II subunit | |
| 170 | TATGTGATTT | | 10.4 | 0.000 | 0.4 | 4.6 | 5216 | ESTs, Highly similar to HSPC028 (H. sapiens) | AA713577, |
| | (SEQ ID NO: 170) | | | | | | | | AA748622, |
| 171 | GGCAGAGGAC | | 10.4 | 0.000 | 0.7 | 7.0 | 118638 | non-metastatic cells I, protein (NM23A) | X17620, AA046312, |
| | (SEQ ID NO: 171) | | | | | | | expressed in | |
| 172 | GCCAAGATGC | | 10.4 | 0.004 | 0.0 | 2.3 | 83135 | p53-responsive gene 6 | AA038471, |
| | (SEQ ID NO: 172) | | | | | | | | AA149617, |
| 173 | CCCACACTAC | | 10.4 | 0.004 | 0.0 | 2.3 | 242024 | guanine nucleotide binding protein (G | M16538, AA548194, |
| | (SEQ ID NO: 173) | | | | | | | protein), beta polypeptide 2 | |
| 174 | CCGTCATCCT | | 10.4 | 0.004 | 0.0 | 2.3 | 153591 | Not56 (D. melanogaster)-like protein | AI914552, |
| | (SEQ ID NO: 174) | | | | | | | | AA412507, |
| 175 | TGAAATAAAC | · | 10.4 | 0.004 | 0.0 | 2.3 | 155212 | methylmalonyl Coenzyme A mutase | AI569812, |
| | (SEQ ID NO: 175) | | | | | | | | AI628986, |
| 176 | TGAAATAAAC | · | 10.4 | 0.004 | 0.0 | 2.3 | 238380 | Human endogenous retroviral protease nRNA, | AA527289 |
| | (SEQ ID NO: 176) | | | | | | | complete cds | |
| 177 | GACCCTGCCC | | 10.4 | 0.004 | 0.0 | 2.3 | 173464 | FK506-binding protein 8 (38 kD) | L37033, AA587607, |
| | (SEQ ID NO: 177) | | | | | | | | |
| 178 | CAGCAGAAGC | | 9.8 | 0.000 | 0.9 | 8.8 | 256313 | pinin, desmosome associated protein | N76807 |
| | (SEQ ID NO: 178) | | | | | | | | |
| 179 | CTCATAGCAG | · | 9.3 | 0.001 | 0.4 | 4.2 | 103636 | chromosome 1 open reading frame 9 | AI312752, |
| | (SEQ ID NO: 179) | | | | | | | | AI312755, |
| 180 | CTCATAGCAG | · | 9.3 | 0.001 | 0.4 | 4.2 | 119252 | tumor protein, translationally-controlled 1 | N92214, AA045631, |
| | (SEQ ID NO: 180) | | | | | | | | |
| 181 | CTGGGCCTGG | | 8.3 | 0.010 | 0.0 | 1.9 | 74573 | similar to vaccinia virus HindIII K4L ORF | N99342, D45708, |
| | (SEQ ID NO: 181) | | | | | | | | |
| 182 | GGGGTAAGAA | | 8.3 | 0.010 | 0.0 | 1.9 | 80423 | prostatic binding protein | AA613924, |
| | (SEQ ID NO: 182) | | | | | | | | AA825254, |
| 183 | GGGCTGGGCC | | 8.3 | 0.010 | 0.0 | 1.9 | 100071 | 6-phosphogluconolactonase | AI217069, |
| | (SEQ ID NO: 183) | | | | | | | | AI279087, |
| 184 | TTTTTTGTAA | | 8.3 | 0.010 | 0.0 | 1.9 | 97858 | SH3-domain binding protein 1 | AA748769 |
| | (SEQ ID NO: 184) | | | | | | | | |
| 185 | GCAGTGGCCT | | 8.3 | 0.010 | 0.0 | 1.9 | 184276 | solute carrier family 9 (sodium/hydrogen | AA425299, |
| | (SEQ ID NO: 185) | | | | | | | exchanger), Isoform 3 regulatory factor 1 | AA593621, |
| 186 | CCCCCAATGC | | 8.3 | 0.010 | 0.0 | 1.9 | 115232 | Spliceosome protein SAP-62 | AI280056, |
| | (SEQ ID NO: 186) | | | | | | | | AI348200, |
| 187 | CTGCTGTGAT | · | 8.3 | 0.010 | 0.0 | 1.9 | 1063 | small nuclear ribonucleoprotein polypeptide C | AA089406, |
| | (SEQ ID NO: 187) | | | | | | | | AA099919, |
| 188 | CAGTTGGTTG | | 8.3 | 0.010 | 0.0 | 1.9 | 155218 | E1B-55 kDa-associated protein 5 | AA130531, |
| | (SEQ ID NO: 188) | | | | | | | | AA155800, |
| 189 | ATCCATAGTG | | 8.3 | 0.010 | 0.0 | 1.9 | 66772 | TATA box binding protein (TBP)-associated | AA662359, |
| | (SEQ ID NO: 189) | | | | | | | factor, RNA polymerase II, N, 68 kD (RNA - | AA857343, |
| | | | | | | | | binding | |
| 190 | CTGGATGCCG | | 8.3 | 0.010 | 0.0 | 1.9 | 106061 | RD RNA-binding protein | AA569818, |
| | (SEQ ID NO: 190) | | | | | | | | AA988602, |
| 191 | CTGACCCCCT | | 8.3 | 0.010 | 0.0 | 1.9 | 26492 | beta-1,3-glucoronyltransferase 3 | AB009598, |
| | (SEQ ID NO: 191) | | | | | | | (glucuronosyltransferase I) | AJ005865, |
| 192 | CCTGTACCCC | · | 8.3 | 0.010 | 0.0 | 1.9 | 32317 | Sox-like transcriptional factor | AA045957, |
| | (SEQ ID NO: 192) | | | | | | | | AA196459, |
| 193 | TAAAATTTGT | · | 8.3 | 0.010 | 0.0 | 1.9 | 32317 | Sox-like transcriptional factor | AA919117 |
| | (SEQ ID NO: 193) | | | | | | | | |
| 194 | TGGCCTGCCC | | 8.3 | 0.010 | 0.0 | 1.9 | 181002 | MLL septin-like fusion | AA761307, |
| | (SEQ ID NO: 194) | | | | | | | | AA831791, |
| 195 | TGGCCTCCCC | | 8.3 | 0.010 | 0.0 | 1.9 | 159161 | Rhp GDP dissociation Inhibitor (GDI) alpha | X69550 |
| | (SEQ ID NO: 195) | | | | | | | | |
| 196 | TGCAGCGCCT | | 8.3 | 0.010 | 0.0 | 1.9 | 77573 | uridine phosphorylase | X90858, AI018589, |
| | (SEQ ID NO: 196) | | | | | | | | |
| 197 | TGAGGGGTGA | | 8.3 | 0.010 | 0.0 | 1.9 | 252979 | G protein pathway suppressor 1 | AA521025, |
| | (SEQ ID NO: 197) | | | | | | | | AA569807, |
| 198 | TGAGGCCAGG | · | 8.3 | 0.010 | 0.0 | 1.9 | 79162 | structure specific recognition protein 1 | AW328290, M85737, |
| | (SEQ ID NO: 198) | | | | | | | | |
| 199 | GAGAGAAGAG | | 8.3 | 0.010 | 0.0 | 1.9 | 13476 | UDP-GalbetaGlcNAc beta 1,4- | AA721091, |
| | (SEQ ID NO: 199) | | | | | | | galactosyltransferase, polypeptide 3 | AA743639, |
| 200 | TCTTCTCACA | · | 8.3 | 0.010 | 0.0 | 1.9 | 656 | cell division cycle 25C | AA206499, |
| | (SEQ ID NO: 200) | | | | | | | | AA534482, |
| 201 | GCCGCTACTT | | 8.3 | 0.010 | 0.0 | 1.9 | 32989 | calcitonin receptor-like receptor activity | AI951585, |
| | (SEQ ID NO: 201) | | | | | | | modifying protein 1 | AJ001014, |
| 202 | CCCTCCTCCG | | 8.3 | 0.010 | 0.0 | 1.9 | 81131 | guanidinoacetate N-methyltransferase | D59710, AI123221, |
| | (SEQ ID NO: 202) | | | | | | | | |
| 203 | TATGACCACA | · | 8.3 | 0.010 | 0.0 | 1.9 | 6650 | vacuolar protein sorting 45B (yeast homolog) | AA765898, |
| | (SEQ ID NO: 203) | | | | | | | | AA769317, |
| 204 | TACATTCACC | | 8.3 | 0.010 | 0.0 | 1.9 | 82043 | D123 gene product | D14876, U27112, |
| | (SEQ ID NO: 204) | | | | | | | | |
| 205 | AAGCGGGACC | | 8.3 | 0.010 | 0.0 | 1.9 | 153436 | N-acetyltransferase, homolog of S. cerevisiae | X77588, AA158247, |
| | (SEQ ID NO: 205) | | | | | | | ARDI | |
| 206 | GATCAATGGA | · | 8.3 | 0.010 | 0.0 | 1.9 | 3090 | EphB1 | AA449788, |
| | (SEQ ID NO: 206) | | | | | | | | AA640161, |
| 207 | GATCAATGGA | · | 8.3 | 0.010 | 0.0 | 1.9 | 251788 | glucosamine-6-phosphate deaminase | AA031910, |
| | (SEQ ID NO: 207) | | | | | | | | AA151768, |
| 208 | GCCGCCATCT | | 8.3 | 0.010 | 0.0 | 1.9 | 89643 | transkelolase (Wernicke-Korsakoff syndrome) | U55017 |
| | (SEQ ID NO: 208) | | | | | | | | |
| 209 | CCCTGGGTTC | | 7.7 | 0.000 | 2.5 | 19.0 | 111334 | ferritin, light polypeptide | M11147, M12938, |
| | (SEQ ID NO: 209) | | | | | | | | |
| 210 | CAGCCTTGGA | | 7.3 | 0.007 | 0.4 | 3.2 | 65648 | RNA binding motif protein 8 | AA045586, |
| | (SEQ ID NO: 210) | | | | | | | | AA188655, |
| 211 | ACCCTTCCCT | · | 7.3 | 0.007 | 0.4 | 3.2 | 99528 | ESTs, Weakly similar to VON EBNER'S | AA936288, |
| | (SEQ ID NO: 211) | | | | | | | GLAND PROTEIN PRECURSOR | AA977608, |
| | | | | | | | | (H. sapiens) | |
| 212 | GAGGGGAAAC | | 7.3 | 0.007 | 0.4 | 3.2 | 81972 | SHC (Src homology 2 domain-containing) | AA767918, X68148, |
| | (SEQ ID NO: 212) | | | | | | | transforming protein 1 | |
| 213 | ACCCTTCCCT | · | 7.3 | 0.007 | 0.4 | 3.2 | 74564 | signal sequence receptor, beta (translocon- | AW024384, D37991, |
| | (SEQ ID NO: 213) | | | | | | | associated protein beta) | |
| 214 | ACTGGGTCTA | | 6.5 | 0.000 | 1.6 | 10.2 | 250871 | non-metastatic cells 2, protein (NM23B) | AA514798, |
| | (SEQ ID NO: 214) | | | | | | | expressed in | AA828464, |
| 215 | TGGAGTGGAG | | 5.8 | 0.000 | 1.1 | 6.5 | 3764 | guanylate kinase 1 | F25667, AA024959, |
| | (SEQ ID NO: 215) | | | | | | | | |
| 216 | CCCCTCCCTC | · | 5.5 | 0.008 | 0.7 | 3.7 | 79410 | solute carrier family 4, anion exchanger, | AA741103, |
| | (SEQ ID NO: 216) | | | | | | | member 2 (erythrocyte membrane protein | AA767203, |
| | | | | | | | | band 3-) | |
| 217 | CCCCTCCCTC | · | 5.5 | 0.008 | 0.7 | 3.7 | 74564 | signal sequence receptor, beta (translocon- | AW083845 |
| | (SEQ ID NO: 217) | | | | | | | associated protein beta) | |
| 218 | ACAGTGGGGA | | 4.6 | 0.003 | 1.1 | 5.1 | 75839 | zinc finger protein 6 (CMPXI) | AA740738, |
| | (SEQ ID NO: 218) | | | | | | | | AA100363, |
| 219 | TGATTTCACT | · | 4.1 | 0.000 | 4.0 | 16.7 | 24322 | ATPase, H+ transporting, lysosomal (vacuolar | AI065143 |
| | (SEQ ID NO: 219) | | | | | | | proton pump) 9 kD | |
| 220 | TTATGGGATC | | 4.0 | 0.000 | 3.4 | 13.5 | 5862 | guanine nucleotide binding protein (G | M24194, AA480431, |
| | (SEQ ID NO: 220) | | | | | | | protein), beta polypeptide 2-like 1 | |
| 221 | ATAGACATAA | | 2.8 | 0.007 | 2.5 | 7.0 | 74614 | complement component 1, q subcomponent | AI916184, |
| | (SEQ ID NO: 221) | | | | | | | binding protein | AA195312, |
| 222 | CACCTAATTG | · | 2.3 | 0.000 | 34.2 | 79.3 | 181368 | U5 anRNP-specific protein (220 kD), ortholog | AW129234, |
| | (SEQ ID NO: 222) | | | | | | | of S. cerevisiae Prp8p | AW151854, |
| 223 | GAAATACAGT | · | −1.8 | 0.006 | 18.6 | 10.2 | 79572 | cathepain D (lysosomal aspartyl protease) | AA046688, |
| | (SEQ ID NO: 223) | | | | | | | | AA063376, |
| 224 | GCCTTCCAAT | | −2.0 | 0.004 | 15.9 | 7.9 | 76053 | DEAD/H (Asp-Glu-Ala-Asp/His) box | AA030969, |
| | (SEQ ID NO: 224) | | | | | | | polypeptide 5 (RNA helicase, 68 kD) | AW019938, |
| 225 | GTGTGTTTGT | | −2.0 | 0.005 | 15.0 | 7.4 | 118787 | transforming growth factor, beta-induced, | M77349, AW021500, |
| | (SEQ ID NO: 225) | | | | | | | 68 kD | |
| 226 | AGCAGATCAG | | −2.2 | 0.004 | 13.2 | 6.0 | 119301 | S100 calcium-binding protein A10 (annexin II | AW022967, |
| | (SEQ ID NO: 226) | | | | | | | ligand, calpactin I, light polypeptide (p11)) | AA009605, |
| 227 | CTGCCAAGTT | | −2.6 | 0.003 | 10.3 | 3.7 | 75873 | zyxin | AA040172, |
| | (SEQ ID NO: 227) | | | | | | | | AA054721, |
| 228 | TTCTGTGAAT | · | −3.7 | 0.002 | 8.5 | 2.3 | 182183 | caldesmon 1 | AA552208, |
| | (SEQ ID NO: 228) | | | | | | | | AA652809, |
| 229 | CTTAATCCTG | | −4.1 | 0.000 | 15.2 | 3.7 | 234433 | ESTs, Weakly similar to transporter protein | AA864787, |
| | (SEQ ID NO: 229) | | | | | | | (H. sapiens) | AW021494, |
| 230 | TCTCAATTCT | · | −4.5 | 0.003 | 6.3 | 1.4 | 173497 | Sec23 (S. cerevisiae) homolog B | AI581164, |
| | (SEQ ID NO: 230) | | | | | | | | AW135796, |
| 231 | GCCCTTTCTC | | −4.5 | 0.000 | 12.5 | 2.8 | 7835 | endocytic receptor (macrophage mannose | AA126747, |
| | (SEQ ID NO: 231) | | | | | | | receptor family) | AA405572, |
| 232 | TCTCAATTCT | · | −4.5 | 0.003 | 6.3 | 1.4 | N/A | BB1 | AA410935, |
| | (SEQ ID NO: 232) | | | | | | | | AA022580, |
| 233 | TTCTTGTTTT | | −5.3 | 0.006 | 4.9 | 0.9 | 74621 | prion protein (p27-30) Creutzfeld-Jakob | D00015, M13687, |
| | (SEQ ID NO: 233) | | | | | | | disease, Gertsmann-Strausler-Scheinker | |
| | | | | | | | | syndrome | |
| 234 | TATGACTTAA | · | −5.3 | 0.006 | 4.9 | 0.9 | 89230 | potassium intermediate/small conductance | AA285078, |
| | (SEQ ID NO: 234) | | | | | | | calcium-activated channel, subfamily N, | AA491238, |
| | | | | | | | | member | |
| 235 | GTTTTTTTTA | · | −5.5 | 0.004 | 5.1 | 0.9 | 10114 | ESTs, Weakly similar to protein B (H. sapiens) | AA284721, |
| | (SEQ ID NO: 235) | | | | | | | | AA496717, |
| 236 | GTCACAGTCC | | −5.8 | 0.009 | 2.7 | 0.0 | 155321 | serum response factor (c-los serum response | AA024483, |
| | (SEQ ID NO: 236) | | | | | | | element-binding transcription factor) | AA041538, |
| 237 | TAAGAAAATG | | −8.3 | 0.008 | 2.9 | 0.0 | 75929 | cadherin 11 (OB-cadherin, osteoblast) | AA258422, |
| | (SEQ ID NO: 237) | | | | | | | | AA461076, |
| 238 | TTTTTTAAAA | | −6.3 | 0.006 | 2.9 | 0.0 | 227400 | mitogen-activated protein kinase kinase kinase | AA043537 |
| | (SEQ ID NO: 238) | | | | | | | kinase 3 | |
| 239 | TTACTTATAC | · | −8.8 | 0.004 | 3.1 | 0.0 | 159 | tumor necrosis factor receptor superfamily, | AW138039 |
| | (SEQ ID NO: 239) | | | | | | | member 1A | |
| 240 | TTACTTATAC | · | −8.8 | 0.004 | 3.1 | 0.0 | 29335 | wingless-type MMTV integration site family, | AW129457 |
| | (SEQ ID NO: 240) | | | | | | | member 2B | |
| 241 | GCTGTTTTGT | | −6.8 | 0.004 | 3.1 | 0.0 | 92186 | KIAA0989 protein | AB023206, |
| | (SEQ ID NO: 241) | | | | | | | | AA405541, |
| 242 | CTTTCTTTGA | | −8.2 | 0.001 | 3.8 | 0.0 | 4909 | regulated in glioma | AF052161, |
| | (SEQ ID NO: 242) | | | | | | | | AA209488, |
| 243 | AAAAGATACT | | −8.2 | 0.009 | 3.8 | 0.5 | 82071 | Cbp/p300-interacting transactivator, with | AA115949, |
| | (SEQ ID NO: 243) | | | | | | | Glu/Asp-rich carboxy-terminal domain, 2 | AA146987, |
| 244 | TCCGTGGTTG | | −8.2 | 0.001 | 3.8 | 0.0 | 79516 | brain acid-soluble protein 1 | AF039656, |
| | (SEQ ID NO: 244) | | | | | | | | AA602987, |
| 245 | CATTATAACT | | −8.2 | 0.001 | 3.8 | 0.0 | 84359 | hypothetical protein | AA806434, |
| | (SEQ ID NO: 245) | | | | | | | | AA832337, |
| 246 | TGTCATCACA | | −9.2 | 0.004 | 4.3 | 0.5 | 83354 | lysyl oxidase-like 2 | AA126278, |
| | (SEQ ID NO: 246) | | | | | | | | AA149435, |
| 247 | TTTTGTTTTG | · | −9.7 | 0.003 | 4.5 | 0.5 | 95583 | transmembrane 4 superfamily member | AA037844, |
| | (SEQ ID NO: 247) | | | | | | | (tetraspan NET-7) | AA040421, |
| 248 | TACAGAGGGA | | −10.1 | 0.002 | 4.7 | 0.5 | 3776 | zinc finger protein 216 | AA730188, |
| | (SEQ ID NO: 248) | | | | | | | | AA814563, |
| 249 | AAGTGAAACA | | −11.1 | 0.001 | 5.1 | 0.5 | 93659 | protein disulfide isomerase related protein | AW411440 |
| | (SEQ ID NO: 249) | | | | | | | (calcium-binding protein, intestinal-related) | |
| 250 | AGTTTCCCAA | | −13.0 | 0.000 | 6.0 | 0.5 | 75854 | SULT1C sulfotransferase | AF055584, |
| | (SEQ ID NO: 250) | | | | | | | | AA113827, |
| 251 | TACAATAAAC | | −14.0 | 0.000 | 6.5 | 0.0 | 9071 | progesterone membrane binding protein | AA836144, |
| | (SEQ ID NO: 251) | | | | | | | | AJ002030, |
| 252 | CATATCATTA | | −15.9 | 0.000 | 7.4 | 0.0 | 119205 | insulin-like growth factor binding protein 7 | AL036223, |
| | (SEQ ID NO: 252) | | | | | | | | AA745836, |
| | SECTION 7. | | | | | | | EST clones of unknown function | |
| 253 | GCACCTCAGC | | 14.5 | 0.000 | 0.0 | 3.2 | 10702 | ESTs | AA027098, |
| | (SEQ ID NO: 253) | | | | | | | | AA035781, |
| 254 | GAGAGAAAAT | | 14.5 | 0.000 | 0.0 | 3.2 | 181444 | ESTs, Weakly similar to R12C12.6 (C. | AA476914, |
| | (SEQ ID NO: 254) | | | | | | | elegans) | AA630706, |
| 255 | ACTTTTTAAA | | 14.1 | 0.000 | 1.1 | 15.8 | 157300 | EST | AI365306 |
| | (SEQ ID NO: 255) | | | | | | | | |
| 256 | TTTCAGGGGA | · | 12.4 | 0.006 | 0.2 | 2.8 | 3804 | DKFZP564C1940 protein | AA831451, |
| | (SEQ ID NO: 256) | | | | | | | | AA070917, |
| 257 | TGATGGGCAT | | 12.4 | 0.006 | 0.2 | 2.8 | 74284 | ESTs, Moderately similar to S. cerevisiae | AI688503, |
| | (SEQ ID NO: 257) | | | | | | | hypothetical protein L3111 (H. sapiens) | AI745361, |
| 258 | CTGGGCGTGT | · | 12.4 | 0.001 | 0.0 | 2.8 | 108948 | ESTs, Weakly similar to laminin beta-2 chain | AA046403, |
| | (SEQ ID NO: 258) | | | | | | | precursor (H. sapiens) | AA759123, |
| 259 | ACGGTGATGT | | 12.4 | 0.001 | 0.0 | 2.8 | 10453 | ESTs | AA005401, |
| | (SEQ ID NO: 259) | | | | | | | | AA045808, |
| 260 | CTGGGCGTGT | · | 12.4 | 0.001 | 0.0 | 2.8 | 15246 | EST | T90794 |
| | (SEQ ID NO: 260) | | | | | | | | |
| 261 | TGAGGCCAGG | · | 8.3 | 0.010 | 0.0 | 1.9 | 110128 | ESTs | AA584364 |
| | (SEQ ID NO: 261) | | | | | | | | |
| 262 | GGTTTGTGTG | | 8.3 | 0.010 | 0.0 | 1.9 | 83954 | Homo sapiens unknown mRNA | AF061739, |
| | (SEQ ID NO: 262) | | | | | | | | AA732389, |
| 263 | TTGTGGGATC | | 8.3 | 0.010 | 0.0 | 1.9 | 167795 | ESTs | AA018907, |
| | (SEQ ID NO: 263) | | | | | | | | AA126960, |
| 264 | CCTGTACCCC | · | 6.3 | 0.010 | 0.0 | 1.9 | 12342 | Homo sapiens clone 24538 mRNA sequence | AI924428 |
| | (SEQ ID NO: 264) | | | | | | | | |
| 265 | TATGACCACA | · | 6.3 | 0.010 | 0.0 | 1.9 | 176577 | ESTs | AI651376 |
| | (SEQ ID NO: 265) | | | | | | | | |
| 266 | ACTACCTTCA | | 8.3 | 0.010 | 0.0 | 1.9 | 9601 | ESTs, Highly similar to CGI-106 protein (H. | AA744772, |
| | (SEQ ID NO: 266) | | | | | | | sapiens) | AA805690, |
| 267 | CTGCTGTGAT | · | 8.3 | 0.010 | 0.0 | 1.9 | 193909 | ESTs, Weakly similar to IIII ALU | AA628209, |
| | (SEQ ID NO: 267) | | | | | | | SUBFAMILY SC WARNING ENTRY IIII | AI690704, |
| | | | | | | | | (H. sapiens) | |
| 268 | GAGTGAGTGA | · | 7.3 | 0.007 | 0.4 | 3.2 | 52186 | ESTs, Weakly similar to IIII ALU | H62203 |
| | (SEQ ID NO: 268) | | | | | | | SUBFAMILY J WARNING ENTRY IIII (H. | |
| | | | | | | | | sapiens) | |
| 269 | GAGTGAGTGA | · | 7.3 | 0.007 | 0.4 | 3.2 | 10483 | ESTs, Weakly similar to C44C1.2 gene | AA009696, |
| | (SEQ ID NO: 269) | | | | | | | product (C. elegans) | AA088448, |
| 270 | TTTGTTAAAA | · | 4.1 | 0.007 | 1.1 | 4.6 | 111244 | ESTs | AA748351, |
| | (SEQ ID NO: 270) | | | | | | | | AA769191, |
| 271 | AAGATAATGC | · | 4.1 | 0.007 | 1.1 | 4.6 | 102898 | ESTs, Weakly similar to C11D2.4 (C. | AA806449, |
| | (SEQ ID NO: 271) | | | | | | | elegans) | AA115687, |
| 272 | AAGATAATGC | · | 4.1 | 0.007 | 1.1 | 4.6 | 251978 | EST | C14037 |
| | (SEQ ID NO: 272) | | | | | | | | |
| 273 | TTTGTTAAAA | · | 4.1 | 0.007 | 1.1 | 4.6 | 207118 | EST | AI806514 |
| | (SEQ ID NO: 273) | | | | | | | | |
| 274 | AGGAAAGCTG | | 4.0 | 0.000 | 6.3 | 25.0 | 76437 | DKFZP566B023 protein | AA523344, |
| | (SEQ ID NO: 274) | | | | | | | | AA551986, |
| 275 | CAAGCATCCC | | 3.5 | 0.000 | 5.8 | 20.4 | 153423 | ESTs | AW275649, |
| | (SEQ ID NO: 275) | | | | | | | | AW276934, |
| 276 | GAAATACAGT | · | −1.8 | 0.006 | 18.6 | 10.2 | 67201 | ESTs | AA122047, |
| | (SEQ ID NO: 276) | | | | | | | | AA404659, |
| 277 | TTCTGTGAAT | · | −3.7 | 0.002 | 8.5 | 2.3 | 77870 | ESTs | AA022926, |
| | (SEQ ID NO: 277) | | | | | | | | AA121431, |
| 278 | GAAATAATGG | · | −4.0 | 0.001 | 9.2 | 2.3 | 178053 | ESTs | AA412270, |
| | (SEQ ID NO: 278) | | | | | | | | AI208372, |
| 279 | GAAATAATGG | · | −4.0 | 0.001 | 9.2 | 2.3 | 209037 | ESTs | AI143898, AI808260 |
| | (SEQ ID NO: 279) | | | | | | | | |
| 280 | TATGACTTAA | · | −5.3 | 0.006 | 4.9 | 0.9 | 203352 | ESTs | W52993 |
| | (SEQ ID NO: 280) | | | | | | | | |
| 281 | GTAAGATTAG | · | −5.8 | 0.000 | 8.1 | 1.4 | 250705 | ESTs | AA953513, |
| | (SEQ ID NO: 281) | | | | | | | | AI129290, |
| 282 | GCCATATTAT | | −5.8 | 0.009 | 2.7 | 0.0 | 19280 | KIAA0544 protein | R70600, AI081100, |
| | (SEQ ID NO: 282) | | | | | | | | |
| 283 | AATTTTCATT | · | −6.3 | 0.006 | 2.9 | 0.0 | 77695 | KIAA0008 gene product | AI889277 |
| | (SEQ ID NO: 283) | | | | | | | | |
| 284 | AATTTTCATT | · | −6.3 | 0.006 | 2.9 | 0.0 | 35092 | ESTs | AA343561, |
| | (SEQ ID NO: 284) | | | | | | | | AA449315, |
| 285 | TTCCTCCTTT | · | −7.7 | 0.002 | 3.6 | 0.0 | 226144 | ESTs | AI215617, |
| | (SEQ ID NO: 285) | | | | | | | | AI982565, |
| 286 | GCTTGTCTTT | | −7.7 | 0.002 | 3.6 | 0.0 | 224620 | ESTs | AI560377, AI953171 |
| | (SEQ ID NO: 286) | | | | | | | | |
| 287 | ACAGTGTTAA | | −7.7 | 0.002 | 3.6 | 0.0 | 166342 | ESTs | AA167571, |
| | (SEQ ID NO: 287) | | | | | | | | AA653213, |
| 288 | TTCCTCCTTT | · | −7.7 | 0.002 | 3.6 | 0.0 | 115581 | EST, Highly similar to KIAA0826 protein (H. | AA463908 |
| | (SEQ ID NO: 288) | | | | | | | sapiens) | |
| 289 | TTTTGTTTTG | · | −9.7 | 0.003 | 4.5 | 0.5 | 74867 | ESTs | AA120954, |
| | (SEQ ID NO: 289) | | | | | | | | AA489661, |
| 290 | ACAGATTTGA | | −10.1 | 0.000 | 4.7 | 0.0 | 41271 | ESTs | AA253217, |
| | (SEQ ID NO: 290) | | | | | | | | AA593446, |
| 291 | TTCCCCCTTC | | −10.6 | 0.000 | 4.9 | 0.0 | 163928 | ESTs | AA577100, |
| | (SEQ ID NO: 291) | | | | | | | | AI819034 |
| 292 | AAAGTCATTG | · | −11.1 | 0.001 | 5.1 | 0.5 | 21145 | Human BAC clone RGOB3M05 from 7q21-7q22 | AI056386, AI161119 |
| | (SEQ ID NO: 292) | | | | | | | | |
| | SECTION 8. | | | | | | | UNIDENTIFIED TRANSCRIPTS | |
| 293 | CCGCCGAAGT | | 24.9 | 0.000 | 0.0 | 5.6 | | | |
| | (SEQ ID NO: 293) | | | | | | | | |
| 294 | ACCTTTTCAA | | 16.6 | 0.000 | 0.0 | 3.7 | | | |
| | (SEQ ID NO: 294) | | | | | | | | |
| 295 | GCCCCTCCGG | | 16.6 | 0.001 | 0.2 | 3.7 | | | |
| | (SEQ ID NO: 295) | | | | | | | | |
| 296 | GCTTTCTCAC | | 10.4 | 0.000 | 0.9 | 9.3 | | | |
| | (SEQ ID NO: 296) | | | | | | | | |
| 297 | CGCCGCGGCT | | 10.4 | 0.004 | 0.0 | 2.3 | | | |
| | (SEQ ID NO: 297) | | | | | | | | |
| 298 | GTGACCACGG | | 9.3 | 0.001 | 0.4 | 4.2 | | | |
| | (SEQ ID NO: 298) | | | | | | | | |
| 299 | GTTAACAGTC | | 8.3 | 0.010 | 0.0 | 1.9 | | | |
| | (SEQ ID NO: 299) | | | | | | | | |
| 300 | GCCGTTCTTA | | 8.3 | 0.010 | 0.0 | 1.9 | | | |
| | (SEQ ID NO: 300) | | | | | | | | |
| 301 | AGGCTACCGG | | 7.3 | 0.007 | 0.4 | 3.2 | | | |
| | (SEQ ID NO: 301) | | | | | | | | |
| 302 | ACTTTTTCAC | | 5.4 | 0.000 | 1.8 | 9.7 | | | |
| | (SEQ ID NO: 302) | | | | | | | | |
| 303 | TCCCCGTCAT | | −4.5 | 0.003 | 6.3 | 1.4 | | | |
| | (SEQ ID NO: 303) | | | | | | | | |
| 304 | AGGAATGTTA | | −5.1 | 0.003 | 4.7 | 0.9 | | | |
| | (SEQ ID NO: 304) | | | | | | | | |
| 305 | TCCCTTATTA | | −5.3 | 0.005 | 4.9 | 0.9 | | | |
| | (SEQ ID NO: 305) | | | | | | | | |
| 306 | TCTTGATATT | | −5.8 | 0.009 | 2.7 | 0.0 | | | |
| | (SEQ ID NO: 306) | | | | | | | | |
| 307 | AAGGCAATTT | | −6.8 | 0.001 | 6.3 | 0.9 | | | |
| | (SEQ ID NO: 307) | | | | | | | | |
| 308 | TTCGGTTGGT | | −7.2 | 0.000 | 10.1 | 1.4 | | | |
| | (SEQ ID NO: 308) | | | | | | | | |
| 309 | TCCCCGGTAC | | −7.5 | 0.000 | 13.9 | 1.9 | | | |
| | (SEQ ID NO: 309) | | | | | | | | |
| 310 | GCTGACGTCA | | −8.2 | 0.001 | 3.8 | 0.0 | | | |
| | (SEQ ID NO: 310) | | | | | | | | |
| 311 | TCCCCCGTAC | | −8.7 | 0.001 | 4.0 | 0.0 | | | |
| | (SEQ ID NO: 311) | | | | | | | | |
| 312 | ACGTTCTCTT | | −9.2 | 0.001 | 4.3 | 0.0 | | | |
| | (SEQ ID NO: 312) | | | | | | | | |
| 313 | TAACTTTTGG | | −9.2 | 0.001 | 4.3 | 0.0 | | | |
| | (SEQ ID NO: 313) | | | | | | | | |
| 314 | AAATGCTTGG | | −9.7 | 0.003 | 4.5 | 0.5 | | | |
| | (SEQ ID NO: 314) | | | | | | | | |
| 315 | CTAAAAACCT | | −10.1 | 0.000 | 4.7 | 0.0 | | | |
| | (SEQ ID NO: 315) | | | | | | | | |
| 316 | GTGAGAGTTT | | −12.1 | 0.000 | 5.6 | 0.5 | | | |
| | (SEQ ID NO: 316) | | | | | | | | |
| 317 | GGTGGACACG | | −13.0 | 0.000 | 6.0 | 0.0 | | | |
| | (SEQ ID NO: 317) | | | | | | | | |
| 318 | TCCCCTATTA | | −15.4 | 0.000 | 7.2 | 0.0 | | | |
| | (SEQ ID NO: 318) | | | | | | | | |
| 319 | CTTTATTCCA | | −18.3 | 0.000 | 17.0 | 0.9 | | | |
| | (SEQ ID NO: 319) | | | | | | | | |

| No | Tag_Sequence | SHEP-2 | SHEP-21N | P value | Unigene cluster | Unigene description | Accession code |
|---|---|---|---|---|---|---|---|
| 1 | CGACGAGGAG SEQ ID NO: 320 | 1.8 | 7.0 | 0.0087 | 9999 | epithelial membrane protein 3 | |
| 2 | TTTAAAAAAA* SEQ ID NO: 321 | 21.7 | 7.0 | 0.0003 | 97437 | centriole associated protein | |
| 3 | GAATAAATGT SEQ ID NO: 322 | 7.2 | 1.2 | 0.0053 | 8762 | FK506-binding protein 9 (63 kD) | AI088246 |
| 4 | TAAAATAAAA* SEQ ID NO: 323 | 5.0 | 0.0 | 0.0027 | 87100 | ESTs | |
| 5 | TCTTCTGCCA SEQ ID NO: 324 | 9.5 | 1.7 | 0.0021 | 86392 | ESTs | |
| 6 | TGCTTTGGGA SEQ ID NO: 325 | 1.4 | 5.8 | 0.0100 | 84344 | ESTs, Highly similar to CGI-135 protein [H. sapiens] | AA788733 |
| 7 | GGGCGCTGTG SEQ ID NO: 326 | 0.9 | 6.4 | 0.0020 | 8372 | ubiquinol-cytochrome c reductase (6.4 kD) subunit | |
| 8 | TTAGATAAGC* SEQ ID NO: 327 | 4.5 | 12.2 | 0.0036 | 82916 | chaperonin containing TCP1, subunit 6A (zeta 1) | |
| 9 | AGCTCTCCCT SEQ ID NO: 328 | 29.3 | 53.4 | 0.0000 | 82202 | ribosomal protein L17 | AA961432 |
| 10 | CCCTGCCTTG SEQ ID NO: 329 | 0.0 | 3.5 | 0.0052 | 82045 | midkine (neurite growth-promoting factor 2) | |
| 11 | GCACAAGAAG SEQ ID NO: 330 | 9.5 | 19.2 | 0.0037 | 81634 | ATP synthase, H+ transporting, mitochondrial F0 complex, subunit b, isoform 1 | |
| 12 | TTAAAGGCCG SEQ ID NO: 331 | 0.5 | 5.2 | 0.0025 | 79086 | ribosomal protein, mitochondrial, L3 | |
| 13 | AGAAAGATGT SEQ ID NO: 332 | 9.5 | 20.3 | 0.0016 | 78225 | annexin A1 | AA846272 |
| 14 | TTTAAAAAAA* SEQ ID NO: 333 | 21.7 | 7.0 | 0.0003 | 77501 | sarcoglycan, beta (43 kD dystrophin-associated glycoprotein) | |
| 15 | GTAAAAAAAA* SEQ ID NO: 334 | 44.7 | 20.9 | 0.0002 | 77495 | UBX Domain-containing 1 | AI492365 |
| 16 | CTTGATTCCC SEQ ID NO: 335 | 0.9 | 6.4 | 0.0020 | 77266 | qulescin Q6 | |
| 17 | TGTGCTCGGG SEQ ID NO: 336 | 2.3 | 7.6 | 0.0091 | 76847 | KIAA0088 protein | |
| 18 | CAGTCTCTCA SEQ ID NO: 337 | 0.9 | 7.0 | 0.0010 | 76230 | ribosomal protein S10 | |
| 19 | TACGTACTGC SEQ ID NO: 338 | 0.0 | 3.5 | 0.0052 | 76086 | ESTs, Highly, similar to small zinc finger-like protein [H. sapiens] | |
| 20 | GGCAAGCCCC SEQ ID NO: 339 | 9.9 | 40.1 | 0.0000 | 76067 | heat shock 27 kD protein 1 | AI540836 |
| 21 | CGGCTGAATT SEQ ID NO: 340 | 0.9 | 5.8 | 0.0043 | 75888 | phosphogluconate dehydrogenase | |
| 22 | GGCTGGGGGC SEQ ID NO: 341 | 19.4 | 38.3 | 0.0001 | 75721 | profilin 1 | AA903097 |
| 23 | TGCTGGGTGG* SEQ ID NO: 342 | 0.5 | 4.6 | 0.0056 | 754 | folylpolyglutamate synthase | |
| 24 | TCAGATCTTT SEQ ID NO: 343 | 39.7 | 70.9 | 0.0000 | 75344 | ribosomal protein S4, X-linked | AA954645 |
| 25 | AAGAGTTTTG SEQ ID NO: 344 | 0.5 | 5.8 | 0.0010 | 75313 | aido-keto reductase family 1, member B1 (aldose reduclase) | |
| 26 | ACAAATCCTT SEQ ID NO: 345 | 1.8 | 7.0 | 0.0087 | 752 | FK506-binding protein 1A (12 kD) | |
| 27 | AATATGTGGG SEQ ID NO: 346 | 5.4 | 12.8 | 0.0060 | 74649 | cytochrome c oxidase subunit Vlc | AI025170 |
| 28 | GGGTTTTTAT SEQ ID NO: 347 | 5.0 | 11.6 | 0.0100 | 74497 | nuclease sensitive element binding protein 1 | |
| 29 | TCTGCTTACA SEQ ID NO: 348 | 2.7 | 8.1 | 0.0093 | 74267 | ribosomal protein L15 | |
| 30 | TTTAAAAAAA* SEQ ID NO: 349 | 21.7 | 7.0 | 0.0003 | 74088 | early growth response 3 | |
| 31 | CAAACCATCC* SEQ ID NO: 350 | 1.4 | 5.8 | 0.0100 | 65114 | keratin 18 | |
| 32 | CAGACTTTTG* SEQ ID NO: 351 | 5.9 | 12.8 | 0.0100 | 63348 | DKFZP586M121 protein | |
| 33 | TTGGGGTTTC SEQ ID NO: 352 | 40.6 | 74.9 | 0.0000 | 62954 | ferritin, heavy polypeptide 1 | AA866040 |
| 34 | AAGCCTTGCT SEQ ID NO: 353 | 5.4 | 0.6 | 0.0098 | 6289 | growth factor receptor-bound protein 2 [No, now described as small stress protein-like | AI039721 |
| | | | | | | protein HSP22 | |
| 35 | AAGAAACCTT SEQ ID NO: 354 | 1.4 | 5.8 | 0.0100 | 5836 | ESTs, Highly similar to CGI-138 protein [H. sapiens] | |
| 36 | AAGGAAATGA SEQ ID NO: 355 | 4.5 | 0.0 | 0.0043 | 57929 | sllt (Drosophila) homolog 3 | AI741785 |
| 37 | AACTAAAAAA* SEQ ID NO: 356 | 68.6 | 91.8 | 0.0010 | 55921 | glutamyl-prolyl-tRNA synthetase | |
| 38 | ATAATTCTTT SEQ ID NO: 357 | 73.1 | 102.8 | 0.0001 | 539 | ribosomal protein S29 | |
| 39 | GTAAAAAAA* SEQ ID NO: 358 | 44.7 | 20.9 | 0.0002 | 460 | Activating Transcription Factor 3 | AA903193 |
| 40 | TTTTAAAAAT* SEQ ID NO: 359 | 7.7 | 1.7 | 0.0100 | 45033 | lacrimal proline rich protein | |
| 41 | CTCGAATAAA SEQ ID NO: 360 | 4.5 | 0.0 | 0.0043 | 34871 | KIAA0569 gene product | AI338485 |
| 42 | AACTAAAAAA* SEQ ID NO: 361 | 68.6 | 91.8 | 0.0010 | 3297 | ribosomal protein S27a | |
| 43 | GAGGCGATCA SEQ ID NO: 362 | 0.0 | 3.5 | 0.0052 | 30783 | ESTs, Weakly similar to eyelld [D. melanogaster] | |
| 44 | GTGGCTGAAA SEQ ID NO: 363 | 5.0 | 0.0 | 0.0027 | 29797 | ribosomal protein L10 | AI582446 |
| 45 | GTTTCCCCAA* SEQ ID NO: 364 | | | 0.0067 | 281434 | | |
| 46 | GGTGAAGACA SEQ ID NO: 365 | 8.6 | 1.2 | 0.0015 | 26951 | Human mRNA for KIAA0375 gene, complete cds | AI080611 |
| 47 | GCATTTAAAT* SEQ ID NO: 366 | 16.7 | 27.3 | 0.0065 | 250876 | eukaryotic translation elongation factor 1 beta 2 | |
| 48 | TTTTGTATTT SEQ ID NO: 367 | 5.9 | 0.6 | 0.0055 | 250705 | ESTs | |
| 49 | CAGACTTTTG* SEQ ID NO: 368 | 5.9 | 12.8 | 0.0100 | 250501 | TNF? elastin microfibril interface located protein | |
| 50 | GATCCCAACA SEQ ID NO: 369 | 0.5 | 4.6 | 0.0056 | 25 | ATP synthase, H+ transporting, mitochondrial F1 complex, beta polypeptide | |
| 51 | CTGTTGATTG SEQ ID NO: 370 | 16.3 | 26.7 | 0.0070 | 249495 | heterogeneous nuclear ribonucleoprotein A1 | |
| 52 | CACGCAATGC* SEQ ID NO: 371 | 0.5 | 5.8 | 0.0010 | 244 | amino-terminal enhancer of split | AI015996 |
| 53 | TGGTACACGT SEQ ID NO: 372 | 0.9 | 5.8 | 0.0043 | 242463 | keratin 8 | |
| 54 | TTTTAAAAAT* SEQ ID NO: 373 | 7.7 | 1.7 | 0.0100 | 240013 | catechol-O-methyltransferase | |
| 55 | ATTCTCCAGT SEQ ID NO: 374 | 48.8 | 72.6 | 0.0002 | 234518 | ribosomal protein L23 | |
| 56 | CCCTTAGCTT SEQ ID NO: 375 | 13.5 | 3.5 | 0.0011 | 233936 | myosin, light polypeptide, regulatory, non-sarcomeric (20 kD) | AI033904 |
| 57 | GTGGTGGGCG* SEQ ID NO: 376 | 0.9 | 7.0 | 0.0010 | 233694 | ESTs, Weakly similar to ZK1058.5 [C. elegans] | |
| 58 | GCGGGGTACC SEQ ID NO: 377 | 1.4 | 5.8 | 0.0100 | 227823 | pM5 protein | |
| 59 | GGAGAGTACA SEQ ID NO: 378 | 0.0 | 3.5 | 0.0052 | 225939 | slalyltransferase 9 (CMP-NeuAc:lactosylceramide alpha-2,3-slalyltransferase; GM3 | |
| | | | | | | synthase) | |
| 60 | TAAAATAAAA* SEQ ID NO: 379 | 5.0 | 0.0 | 0.0027 | 21254 | TRAF Interacting protein | |
| 61 | GTGGTGGGCG* SEQ ID NO: 380 | 0.9 | 7.0 | 0.0010 | 209741 | EST | |
| 62 | GTGGTGGGCG* SEQ ID NO: 381 | 0.9 | 7.0 | 0.0010 | 208985 | ESTs | |
| 63 | TCCAAATCGA SEQ ID NO: 382 | 6.8 | 1.2 | 0.0080 | 2064 | vimentin | |
| 64 | TAAAATAAAA* SEQ ID NO: 383 | 5.0 | 0.0 | 0.0027 | 204144 | ESTs, Moderately similar to tumor necrosis factor-alpha-induced protein B12 [H. sapiens] | |
| 65 | TAAAATAAAA* SEQ ID NO: 384 | 5.0 | 0.0 | 0.0027 | 202218 | EST | |
| 66 | TTCAATAAAA* SEQ ID NO: 385 | 58.7 | 93.0 | 0.0000 | 2012 | transcobalamin I (vitamin B12 binding protein, R binder family) | AI738761 |
| 67 | TGCTGGGTGG* SEQ ID NO: 386 | 0.5 | 4.6 | 0.0056 | 198273 | NADH dehydrogenase (ubiquinone) 1 beta subcomplex, 8 (19 kD, ASHI) | |
| 68 | TAAAATAAAA* SEQ ID NO: 387 | 5.0 | 0.0 | 0.0027 | 190401 | ESTs, Weakly similar to predicted using Genefinder [C. elegans] | |
| 69 | AAGGTCGAGC SEQ ID NO: 388 | 1.8 | 7.0 | 0.0087 | 184582 | ribosomal protein L24 | |
| 70 | GCATAATAGG SEQ ID NO: 389 | 31.6 | 54.0 | 0.0001 | 184108 | ribosomal protein L21 | AI224420 |
| 71 | GTAAAAAAAA* SEQ ID NO: 390 | 44.7 | 20.9 | 0.0002 | 183842 | Ubiquitin B | AA988708 |
| 72 | TAATATTTTT SEQ ID NO: 391 | 11.7 | 2.9 | 0.0020 | 182485 | actinin, alpha 4 | |
| 73 | GAAAAATGGT SEQ ID NO: 392 | 48.3 | 87.7 | 0.0000 | 181357 | laminin receptor 1 (67 kD, ribosomal protein SA) | AI025931 |
| 74 | AGAACCTTAA SEQ ID NO: 393 | 5.0 | 0.0 | 0.0027 | 181244 | major histocompatibility complex, class I, A | AI023950 |
| 75 | CTCATAAGGA SEQ ID NO: 394 | 49.2 | 70.9 | 0.0006 | 181165 | eukaryotic translation elongation factor 1 alpha 1 | |
| 76 | TAATTTTGGA SEQ ID NO: 395 | 9.0 | 17.4 | 0.0078 | 180152 | ESTs | |
| 77 | CAATAAATGT SEQ ID NO: 396 | 37.0 | 57.5 | 0.0004 | 179779 | ribosomal protein L37 | |
| 78 | TTCAATAAAA* SEQ ID NO: 397 | 58.7 | 93.0 | 0.0000 | 177592 | ribosomal protein, large, P1 | AA961386 |
| 79 | TTAAATAGCA SEQ ID NO: 398 | 6.8 | 0.6 | 0.0021 | 172928 | collagen, type I, alpha 1 | AA992596 |
| 80 | GGAGGAGAGC SEQ ID NO: 399 | 5.0 | 0.0 | 0.0027 | 172928 | collagen, type I, alpha 1 | |
| 81 | TGAAATTGTC SEQ ID NO: 400 | 4.5 | 0.0 | 0.0043 | 172928 | collagen, type I, alpha 1 | |
| 82 | AATGCAGGCA SEQ ID NO: 401 | 1.4 | 6.4 | 0.0067 | 172673 | S-adenosylhomocysteine hydrolase | AI051370 |
| 83 | TTCCGGTTCC SEQ ID NO: 402 | 9.0 | 1.7 | 0.0030 | 172609 | nucleobindin 1 | AI025019 |
| 84 | CACGCAATGC* SEQ ID NO: 403 | 0.5 | 5.8 | 0.0010 | 170683 | EST | AI015996 |
| 85 | TAGACTTATT SEQ ID NO: 404 | 0.5 | 4.6 | 0.0056 | 170197 | glutamic-oxaloacetic transaminase 2, mitochondrial (aspartate aminotransferase 2) | |
| 86 | TTCACAGTGG SEQ ID NO: 405 | 0.5 | 5.2 | 0.0025 | 169992 | protein phosphatase 3 (formerly 2B), regulatory subunit B (19 kD), alpha isoform | |
| | | | | | | (calcineurin B, type I) | |
| 87 | TGCACGTTTT SEQ ID NO: 406 | 37.5 | 61.0 | 0.0001 | 169793 | ribosomal protein L32 | AA922605 |
| 88 | GCAGCTCAGG SEQ ID NO: 407 | 2.3 | 7.6 | 0.0091 | 167137 | ESTs, Moderately similar to CATHEPSIN D PRECURSOR [H. sapiens] | |
| 89 | GGTGAGACAC SEQ ID NO: 408 | 2.7 | 8.7 | 0.0071 | 164280 | solute carrier family 25 (mitochondrial carrier; adenine nucleotide translocator), member 6 | |
| 90 | TTTAAAAAAA* SEQ ID NO: 409 | 21.7 | 7.0 | 0.0003 | 155545 | 37 kDa leucine-rich repeat (LRR) protein | |
| 91 | ATGTCATCAA SEQ ID NO: 410 | 1.4 | 7.0 | 0.0030 | 152936 | adaptor-related protein complex 2, mu 1 subunit | |
| 92 | TAATAAAGGT SEQ ID NO: 411 | 45.1 | 62.2 | 0.0029 | 151604 | ribosomal protein S8 | AI312878 |
| 93 | TTAAAACAAA SEQ ID NO: 412 | 4.1 | 0.0 | 0.0070 | 150508 | ESTs | AI280093 |
| 94 | TGGCCTAAAA SEQ ID NO: 413 | 4.5 | 0.0 | 0.0043 | 1501 | syndecan 2 (heparan sulfate proteoglycan 1, cell surface-associated, fibroglycan) | AI005403 |
| 95 | GGTTAATTGA SEQ ID NO: 414 | 4.1 | 0.0 | 0.0070 | 14376 | | AI590977 |
| 96 | TTCAATAAAA* SEQ ID NO: 415 | 58.7 | 93.0 | 0.0000 | 141269 | ESTs | AA961386 |
| 97 | ATGTGAAGAA SEQ ID NO: 416 | 6.8 | 1.2 | 0.0080 | 13662 | Homo sapiens clone 25036 mRNA sequence | AI279223 |
| 98 | TTTAAAAAAA* SEQ ID NO: 417 | 21.7 | 7.0 | 0.0003 | 134533 | ESTs | |
| 99 | TTAGATAAGC* SEQ ID NO: 418 | 4.5 | 12.2 | 0.0036 | 134350 | ESTs | |
| 100 | GTGACAGAAG SEQ ID NO: 419 | 5.4 | 13.4 | 0.0034 | 129673 | eukaryotic translation Initiation factor 4A, Isoform 1 | |
| 101 | CTGGGTTAAT SEQ ID NO: 420 | 42.4 | 77.8 | 0.0000 | 126701 | ribosomal protein S19 | AA865047 |
| 102 | CAAACCATCC* SEQ ID NO: 421 | 1.4 | 5.8 | 0.0100 | 125170 | ESTs | |
| 103 | GGGACTGGGC* SEQ ID NO: 422 | 0.5 | 5.2 | 0.0025 | 122256 | ESTs | |
| 104 | GCATTTAAAT* SEQ ID NO: 423 | 16.7 | 27.3 | 0.0065 | 120979 | ESTs | |
| 105 | TAGGTTGTCT SEQ ID NO: 424 | 55.1 | 71.5 | 0.0057 | 119252 | tumor protein, translationally-controlled 1 | AA808956 |
| 106 | GACGTGTGGG SEQ ID NO: 425 | 0.0 | 4.6 | 0.0009 | 119192 | H2A histone family, member Z | |
| 107 | GGCTTTACCC SEQ ID NO: 426 | 9.0 | 20.9 | 0.0006 | 119140 | eukaryotic translation initiation factor 5A | |
| 108 | AACTAATACT SEQ ID NO: 427 | 4.5 | 12.8 | 0.0022 | 118724 | DR1-associated protein 1 (negative cofactor 2 alpha) | AI028098 |
| 109 | TAAAATAAAA* SEQ ID NO: 428 | 5.0 | 0.0 | 0.0027 | 118246 | ESTs | |
| 110 | GGGACTGGGC* SEQ ID NO: 429 | 0.5 | 5.2 | 0.0025 | 117848 | hemoglobin, epsilon 1 | |
| 111 | TAAAAACAAA SEQ ID NO: 430 | 5.4 | 0.6 | 0.0098 | 114599 | | AW029321, W1923 |
| 112 | GTTTCCCCAA* SEQ ID NO: 431 | 1.4 | 6.4 | 0.0067 | 112423 | Homo sapiens mRNA; cDNA DKFZp586l1420 (from clone DKFZp586l1420) | |
| 113 | GCAAAAAAAA SEQ ID NO: 432 | 67.3 | 31.4 | 0.0000 | 11221 | ESTs, Weakly similar to fos39554_1 [H. sapiens] | AI057027 |
| 114 | CCAGAACAGA SEQ ID NO: 433 | 37.5 | 55.8 | 0.0011 | 111222 | ribosomal protein L30 | |
| 115 | GTACGGAGAT SEQ ID NO: 434 | 0.5 | 4.6 | 0.0056 | 109225 | vascular cell adhesion molecule 1 | |
| 116 | GTTTCCCCAA* SEQ ID NO: 435 | 1.4 | 6.4 | 0.0067 | 107573 | sialyltransferase | |
| 117 | GGCCCCTCAC SEQ ID NO: 436 | 0.5 | 4.6 | 0.0056 | 106283 | Insulin-like growth factor binding protein 6 | |
| 118 | TTTAAAAAAA* SEQ ID NO: 437 | 21.7 | 7.0 | 0.0003 | 108204 | ESTs, Moderately similar to antigen containing epitope to monoclonal antibody MMS- | |
| | | | | | | 85/12 [M. musculus] | |
| 119 | CCTCCCCCGT SEQ ID NO: 438 | 0.9 | 5.2 | 0.0092 | 10488 | Breakpoint cluster region protein, uterine lelomyoma, 1; barrier to autointegration factor | |
| 120 | AGCAGGGCTC SEQ ID NO: 439 | 0.0 | 5.2 | 0.0004 | 100623 | phospholipase C, beta 3, neighbor pseudogene | |
| 121 | AATTGCAAGC SEQ ID NO: 440 | 5.0 | 0.0 | 0.0027 | — | | U50523 |
| 122 | TTTAACGGCC SEQ ID NO: 441 | 43.8 | 12.8 | 0.0000 | — | | AA196553 |
| 123 | CATTGCCTTC SEQ ID NO: 442 | 5.9 | 0.6 | 0.0055 | — | | |
| 124 | GCTTGCTGCC SEQ ID NO: 443 | 4.5 | 0.0 | 0.0043 | — | | |
| 125 | AAAACATTCT SEQ ID NO: 444 | 67.7 | 32.5 | 0.0000 | — | | AI538076 |
| 126 | GCAGACATTG SEQ ID NO: 445 | 0.9 | 5.8 | 0.0043 | — | | |
| 127 | ACCCTTGGCC SEQ ID NO: 446 | 12.2 | 26.7 | 0.0003 | — | | |
| 128 | AGTAGGTGGC SEQ ID NO: 447 | 8.1 | 1.7 | 0.0068 | — | U5 snRNP-specific protein (220 kD), ortholog of S. cerevisiae Prp8p | |
| 129 | GCAAGCCAAC SEQ ID NO: 448 | 16.7 | 33.1 | 0.0002 | — | actinin, alpha 1 | AI669642 |
| 130 | TTAGCTTGTT SEQ ID NO: 449 | 4.1 | 0.0 | 0.0070 | — | | |
| 131 | GTAATAACTT SEQ ID NO: 450 | 4.1 | 0.0 | 0.0070 | — | | |
| 132 | CGCCGTCGGC SEQ ID NO: 451 | 0.0 | 3.5 | 0.0052 | — | | |
| 133 | ACTCTTTCAA SEQ ID NO: 452 | 0.5 | 4.6 | 0.0056 | — | | |
| 134 | TGCTACGAAA SEQ ID NO: 453 | 5.9 | 0.6 | 0.0055 | — | | |
| 135 | CCCCGGTACA SEQ ID NO: 454 | 7.2 | 1.2 | 0.0053 | — | | |
| 136 | TTATAAAAGA SEQ ID NO: 455 | 10.8 | 3.5 | 0.0098 | — | | |

What is claimed is:

1. A nucleic acid library, comprising:
a myc-dependent downstream gene or functional fragment thereof;
wherein said myc-dependent downstream gene or functional fragment thereof is capable of supporting a neoplastic character of cancer, said neoplastic character selected from the group consisting of growth, invasion and spread, and mixtures thereof.

2. The nucleic acid library of claim 1, wherein said myc-dependent downstream gene or functional fragment thereof comprises a nucleic acid sequence selected from any one of the Tag sequences of Table 1 or Table 2 or an essential equivalent thereof.

3. The nucleic acid library of claim 1 or 2, wherein said myc-dependent downstream gene encodes a ribosomal protein.

4. The nucleic acid library of claim 1 or 2, wherein said myc-dependent downstream gene encodes a protein related to protein synthesis.

5. The nucleic acid library of claim 1 or 2, wherein said myc-dependent downstream gene encodes a protein related to metastasis.

6. The nucleic acid library of claim 1 or 2, wherein said myc-dependent downstream gene encodes a glycolysis enzyme.

7. The nucleic acid library of claim 1 or 2, wherein said myc-dependent downstream gene encodes a mitochondrial functional protein.

8. A method for modulating a gene capable of supporting an essentially neplastic character of a cancer, said method comprising:
activating or repressing said gene, wherein said gene is under the control of a myc-family transcription factor.

9. The method according to claim 8, further comprising administering a drug to a subject, wherein said drug activates or represses said gene.

10. The method according to claim 8, wherein said myc-dependent downstream gene comprises a nucleic acid sequence selected from any one of the Tag sequences of Table 1 or Table 2, or an essential equivalent thereof.

11. The method according to claim 8 or 9, wherein said cancer comprises a myc expressing tumor cell.

12. The method according to any one of claims 8 to 10, wherein myc comprises N-myc.

13. The method according to claim 11, wherein said cancer comprises neuroblastoma.

14. A method for diagnosing cancer, said method comprising:
detecting the relative presence or absence of a myc-dependent downstream gene or a gene product derive thereof;
wherein said myc-dependent downstream gene or gene product derive thereof is capable of supporting a neoplastic character of said cancer.

15. A method for enhancing the production of a recombinant protein, said method comprising:
inducing expression of the recombinant protein in a protein production system with an endogenous or transfected myc gene.

16. The method according to claim 15, wherein said protein production system is within a eukaryotic cell.

17. A method for identifying a substance capable of interfering with n-myc or n-myc induced modulation of transcripts and/or proteins, said method comprising:
providing a cell with n-myc activity; and
determining the modulation of said transcripts and/or proteins in the presence of said substances.

18. The method according to claim 17, wherein the cell with n-myc activity comprises a nucleic acid encoding said n-myc activity.
